(19) United States
(12) Patent Application Publication
(10) Pub. No.: US 2026/0265256 A1
(43) Pub. Date: Sep. 10, 2026

(54) SUBSTITUTED NITROGEN-CONTAINING TRICYCLIC COMPOUNDS AS PARP INHIBITORS AND THE USE THEREOF

(71) Applicant: IMPACT THERAPEUTICS (SHANGHAI), INC., Pudong New Area, Shanghai (CN)

(72) Inventors: Sui Xiong Cai, Pudong New Area (CN); Ye Edward Tian, Pudong New Area (CN); Xiaozhu Wang, Nanjing, Jiangsu (CN)

(21) Appl. No.: 19/161,071

(22) PCT Filed: Feb. 29, 2024
(86) PCT No.: PCT/CN2024/079373

§ 371 Date: Aug. 29, 2025

(30) Foreign Application Priority Data
Mar. 1, 2023 (CN) 202310188881.3
Sep. 28, 2023 (CN) 202311280259.1

(51) Int. Cl. C07D 487/04; A61K 31/4985; A61P 35/00; C07D 491/048; C07D 495/04; C07D 498/04; C07D 513/04

(52) U.S. Cl. CPC C07D 487/04; A61K 31/4985; A61P 35/00; C07D 491/048; C07D 495/04; C07D 498/04; C07D 513/04

(57) ABSTRACT

The disclosure provides substituted nitrogen containing tricyclic compounds as PARP inhibitors and the use thereof. This disclosure provides compounds represented by Formula (I) as below, wherein, the ring Z, Z₁, Z₂, Z₃, Z₄, Z₅, A₁, A₂, A₃, L, m, n, ring B and Cy are defined herein. The compounds of Formula I of the present disclosure are PARP inhibitors and thus are useful in the treatment of diseases, disorders and conditions, such as cancer, responsive to the inhibition of PARP activity. The present disclosure also relates to a pharmaceutical composition comprising the compound of Formula I and the use of the compound of Formula I in the preparation of a medicament for the treatment or prevention of diseases or conditions responsive to the inhibition of PARP activity.

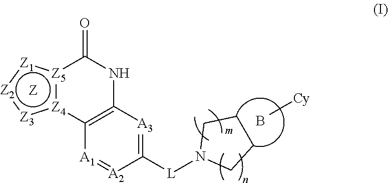

### FIELD OF THE DISCLOSURE

The disclosure relates to substituted nitrogen-containing tricyclic compounds as PARP inhibitors and the use thereof.

### BACKGROUND OF THE INVENTION

Poly(ADP-ribose) polymerase (PARP) is a family of proteins, which transfer negatively charged ADP-ribose groups from donor NAD⁺ onto target proteins. That is one of many post-transcriptional modifications. Therefore, PARP also is termed as ADP-ribose transferase.

Humans are thought to express 17 PARPs identified based on amino acid sequence homology to the catalytic domain (Vyas et al., 2013 Nature Communication, 4: 3240/1-3240/13). PARPs either catalyze the addition of a single ADP-ribose unit on target proteins or catalyze the polymerization of ADP-ribose units to form poly ADP-ribose, also known as poly (ADP-ribose) modification. As a result, the PARP family is further grouped into two subfamilies accordingly. Post-translational modification of poly (ADP-ribose) regulate many aspects of protein function and the physiological function of many PARPs have not been established.

The most characteristic member of the PARP family is PARP1, which was found to have the highest intracellular levels. PARP1 consists of 1014 amino acids (NCBI Accession P09874) with a total molecular weight of approximately 116 kDa. Structurally, this enzyme is composed of two main domains including an N-terminal DNA-binding domain and a catalytic domain. PARP1 is known to play an important role in many cellular functions, including gene expression, transcription, cell division, cell differentiation, cell apoptosis, DNA damage response and repair. PARP1 is activated when DNA damage occurs and is involved in base excision repair (BER) which is a major mechanism of DNA single-strand damage repair. PARP1 binds to the site of Single Strand Break (SSB), and subsequently repair DNA via BER. In response to DNA damage, cells also have evolved two main repair pathways: Homologous Recombination (HR) and Non-Homologous End Joining (NHEJ), in addition to BER repair mechanisms. HR deficient tumors have been found to be sensitive to PARP inhibitors, indicating homologous recombination defects and PARP1 inhibition formed a pair of synthetic lethality, which has been validated by clinical studies. Several PARP inhibitors are currently approved for the treatment of breast, ovarian, pancreatic and prostate cancers with DNA damage repair deficient such as BRCA1/2 mutation. In addition, PARP1 inhibitors may also be used to treat diseases caused by excessive cell death, including central nervous system (CNS) diseases, such as stroke and neurodegenerative disease (Akinori Iwashita et al., 2004, J. Pharmacol. Exp. Thera. 310: 425).

PARP2 is a protein of 559 amino acids with molecular mass of approximately 62 kDa (NCBI Accession NP_005475) and composed of DNA binding domain and catalytic domain (Ame et al., 1999 J Biol Chem 274: 17860-17868). The catalytic domain of PARP2 is very similar to that of PARP1. PARP2 is also found to have similar functions to PARP1 and is involved in the repair of DNA damage through BER mechanism (Schreiber et al., 2002 J Biol Chem 277: 23028-23036). Marketed PARP inhibitors, such as Olaparib, Niraparib, Talazoparib and Rucaparib, not only have inhibitory activities against PARP1, but also have similar inhibitory activities against PARP2. Based on the results of clinical trials, the therapeutic effects of these marketed PARP inhibitors are comparable whereas their toxicity profiles are quite different. For example, Talazoparib has toxicity similar to chemotherapy drugs such as hair loss. A recent study comparing the selectivity of multiple PARP inhibitors showed that Talazoparib not only inhibits PARP1 and PARP2, but also has a relatively high affinity for the other two members of the PARP family, TNKS1 (Tankyrase 1) and TNKS2 (Tankyrase 2) (Ryan et al., 2021, J Biol Chem 296:100251/1-100251/13). The amino acid sequences of TNKS1 and TNKS2 have a high degree of similarity. They share 83% sequence identity overall, and their catalytic domain sequences are 89% identical. They play roles in DNA repair, telomere maintenance, and Wnt/β-catenin signaling. Targeting PARPs other than PARP1 may be the reason why PARP inhibitors cause off-targeted toxicity, such as hair loss and diarrhea. In addition, inhibition of PARP2 activity has been found to lead to hematotoxicity (Farres et al., 2013 Blood 122: 44-54; Farrés et al., 2015 Cell Death and Differentiation 22: 1144-1157). The toxicity of these PARP inhibitors limits their clinical application as well as in combination with other targeted drugs.

Therefore, developing highly selective PAPR1 inhibitors may reduce both mechanism—related or mechanism—independent toxicity.

Multiple selective PARP1 inhibitors have been disclosed, for example, WO2011006803, WO2013014038, WO2021013735, WO2021260092, CN115232129, WO2022225934, WO2022222921, WO2022222964, WO2022222965, WO2022222966, WO2022228387, WO2022247816, WO2022223025, WO2022222995, WO2023122140, WO2023056039, WO2023141290, WO2023146957, WO2023146960 and WO2023156386.

### SUMMARY OF THE DISCLOSURE

The disclosure provides compounds and analogues thereof as represented by Formula I (including Formulae II and III). The compounds can be used as PARP inhibitors. In particular, the compounds of the disclosure are selective PARP1 inhibitors relative to PARP2.

The disclosure also provides pharmaceutical compositions comprising an effective amount of the compound of Formula I (including Formulae II and III). The pharmaceutical compositions can be used for the treatment of cancer.

In a specific embodiment, the pharmaceutical composition may further contain one or more pharmaceutically acceptable carriers, excipients or diluents. The pharmaceutical composition can be used for the treatment of cancer.

In a specific embodiment, the pharmaceutical composition may further contain at least one known anticancer drug or pharmaceutically acceptable salts thereof. The pharmaceutical composition can be used for the treatment of cancer.

The disclosure is also directed to methods for the preparation of novel compounds of Formula I (including Formulae II and III).

### DETAILED DESCRIPTION OF THE DISCLOSURE

It should be understood that the characteristics of the embodiments described herein can be arbitrarily combined to form the technical solution of this disclosure. The definition of each group herein can apply to any of the embodiments described herein. For example, the definitions of the substituents of alkyl herein apply to any of the embodiments described herein unless the substituents of alkyl are clearly defined in the embodiment.

The term “hydrogen (H)” as employed herein includes its isotopes deuterium (D) and tritium (T).

The term “heteroatoms” as employed herein includes oxygen (O), sulfur (S) and nitrogen (N).

The term “alkyl” as used herein refers to alkyl itself or a straight or branched chain radical of up to ten carbons. Useful alkyl groups include straight-chain or branched C₁₋₁₀ alkyl, preferably C₁₋₆ alkyl. In some embodiments, alkyl is C₁₋₄ alkyl. In some embodiments, alkyl is C₁₋₃ alkyl. In some embodiments, alkyl is deuterated C₁₋₃ alkyl. Typical C₁₋₁₀ alkyl groups include methyl, trideuteromethyl, ethyl, propyl, isopropyl, butyl, sec-butyl, tert-butyl, pentyl (such as 3-pentyl), hexyl and octyl groups, which may be optionally substituted.

The term “alkenyl” as used herein refers to a straight or branched chain radical of 2-10 carbon atoms, unless the chain length is limited thereto, there is at least one double bond between two of the carbon atoms in the chain; preferably, the alkenyl is a C₂₋₆ alkenyl, more preferably a C₂₋₄ alkenyl. Typical alkenyl groups include ethenyl, 1-propenyl, 2-propenyl, 2-methyl-1-propenyl, 1-butenyl and 2-butenyl.

The term “alkynyl” as used herein refers to a straight or branched chain radical of 2-10 carbon atoms, unless the chain length is limited thereto, there is at least one triple bond between two of the carbon atoms in the chain; preferably, the alkynyl is a C₂₋₆ alkynyl, more preferably a C₂₋₄ alkynyl. Typical alkynyl groups include ethynyl, 1-propynyl, 1-methyl-2-propynyl, 2-propynyl, 1-butynyl and 2-butynyl.

Useful alkoxy groups include oxygen substituted by the above mentioned C₁₋₁₀ alkyl groups, preferred C₁₋₆ alkyl groups or C₁₋₄ alkyl groups or C₁₋₃ alkyl groups, e.g., methoxy, ethoxy, etc. The alkyl in the alkoxy groups may be optionally substituted. Substituents of alkoxy groups include, without limitation, halogen, morpholino, amino (including alkylamino and dialkylamino), and carboxy (including esters thereof).

Useful amino and optionally substituted amino groups are —NR′R″, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl, an optionally substituted 3-6 membered heterocyclic group or an optionally substituted 5 membered heteroaryl, or R′ and R″ together with the N to which they are attached form an optionally substituted 4-7 membered cyclic amino group, which optionally comprises one or more (such as 2, 3) additional heteroatoms selected from a group consisting of O, N and S. Preferably, the amino groups include —NH₂, —NHR′ and —NR′R″, wherein R′ and R″ are each independently selected from a group consisting of an optionally substituted C₁₋₄ alkyl, an optionally substituted C₃₋₆ cycloalkyl or an optionally substituted 3-6 membered heterocyclic group.

The term “oxo” as used herein refers to ═O.

The term “aryl” as used herein by itself or as part of another group refers to monocyclic, bicyclic or tricyclic aromatic groups containing 6 to 14 carbon atoms. Aryl may be substituted by one or more substituents as described herein.

Useful aryl groups include C₆₋₁₄ aryl groups, preferably C₆₋₁₀ aryl groups. Typical C₆₋₁₄ aryl groups include phenyl, naphthyl, phenanthryl, anthracyl, indenyl, azulyl, biphenyl, biphenylene and fluorenyl.

The term “carbocyclic group” as used herein include cycloalkyl and partially saturated carbocyclic groups. Useful cycloalkyl groups are C₃₋₈ cycloalkyl. Typical cycloalkyl groups include cyclopropyl, cyclobutyl, cyclopentyl, cyclohexyl and cycloheptyl. Useful partially saturated carbocyclic groups are cycloalkenyl, such as C₃₋₈ cycloalkenyl, which include cyclopentenyl, cycloheptenyl and cyclooctenyl. Carbocyclic group may be substituted by one or more substituents as described herein.

Useful halo or halogen groups include fluoro (F), chloro (Cl), bromo (Br) and iodo (I).

Useful acylamino (amido) groups are any C₁₋₆ acyl (alkanoyl) attached to an amino nitrogen, e.g., acetamino, propionamido, butanoylamido, pentanoylamido and hexanoylamido, as well as aryl-substituted C₁₋₆ acylamino groups, e.g., benzoylamido.

Useful acyl groups include C₁₋₆ acyl groups, such as acetyl. Acyl may be optionally substituted by group selected from halo, amino and aryl, wherein the amino and aryl may be optionally substituted. When acyl is substituted by halo, the number of halogen substituents may be in the range of 1-5. Examples of substituted acyls include chloroacetyl and pentafluorobenzoyl. When acyl is substituted by amino, amino group may be substituted by one or two substituents as described herein. In some embodiments, aminoacyl is —C(O)—NR′R″, wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl or an optionally substituted 3-6 membered heterocyclic group.

The term “heterocyclic group” as used herein refers to a saturated or partially saturated 3-7 membered monocyclic, or 7-10 membered bicyclic ring, spirocyclic ring or bridged ring system, which consists of carbon atoms and one to four heteroatoms independently selected from a group consisting of O, N, and S, wherein the nitrogen and/or sulfur heteroatoms can be optionally oxidized and the nitrogen can be optionally quaternized, and the term also includes any bicyclic ring system in which any of the above-defined heterocyclic rings is fused to a benzene ring. The heterocyclic group can be substituted on carbon atom or nitrogen atom if the resulting compound is stable. The heterocyclic group may be substituted by one or more substituents as described herein.

Useful saturated or partially saturated heterocyclic groups include tetrahydrofuranyl, tetrahydropyranyl, piperidinyl, piperazinyl, 1,4-diazepanyl, azetidinyl, oxetanyl, pyrrolidinyl, imidazolidinyl, imidazolinyl, indoline, isoindoline, quinuclidinyl, morpholinyl, isochromanyl, chromanyl, pyrazolidine, pyrazolinyl, tetrahydroisoquinolyl, tetronoyl and tetramoyl, which may be optionally substituted by one or more substituents as described herein.

The term “heteroaryl” as used herein refers to a group having 5 to 14 ring atoms, preferably 5 to 10 ring atoms, with 6, 10 or 14 electrons shared in a cyclic array. Ring atoms are carbon atoms and 1-3 heteroatoms selected from a group consisting of oxygen, nitrogen and sulfur. Heteroaryl may be optionally substituted by one or more substituents as described herein.

Useful heteroaryl groups include thienyl (thiophenyl), benzo[d]isothiazol-3-yl, benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, imidazolyl, pyrazolyl, pyridyl (pyridinyl, including without limitation 2-pyridyl, 3-pyridyl and 4-pyridyl), pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, tetrahydropyridyl, tetrahydrocyclopenta[c]pyrazol-3-yl, benzoisoxazolyl such as 1,2-benzoisoxazol-3-yl, benzimidazolyl, 2-oxindolyl, thiadiazolyl, 2-oxobenzimidazolyl, imidazopyridazinyl, imidazopyridyl, triazolopyridazinyl, tetrahydropyridopyrimidinyl, pyrazolopyrimidinyl, pyrrolopyrimidinyl, pyrrolopyridyl, pyrrolopyrazinyl, triazolopyrazinyl, thiophenoquinolinyl, furanoquinolinyl, thiazoloquinolinyl, pyrazoquinolinyl, pyrroquinolinyl, imidazoquinolinyl, oxazoquinolinyl, etc. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

In this disclosure, unless otherwise described, when substituted, the alkyl, cycloalkyl, heterocycloalkyl, alkoxy, heterocycloalkoxy, alkenyl, heterocycloalkenyl, alkynyl, amino, amido, acyloxy, carboxyl, hydroxyl, mercapto, alkylthio sulfonyl, sulfonyl, sulfinyl, aminoacyl, silyl, phosphinecarboxy, phosphono, carbocyclic group, heterocyclic group, aryl or heteroaryl as described in any embodiment herein may be substituted by one or more (such as 1, 2, 3, 4, 5 or 6) substituents selected from the group consisting of halogen, hydroxyl, carboxyl, amino, nitro, cyano, C₁₋₆ amido, C₁₋₆ acyloxy, C₁₋₆ alkoxy, aryloxy, alkylthio, C₁₋₆ alkyl, C₁₋₆ acyl, C₆₋₁₀ aryl, C₃₋₈ cycloalkyl, C₂₋₆ alkenyl, C₂₋₆ alkynyl, heterocyclic or heteroaryl, methylenedioxy, urea group, mercapto group, azide group, carbonyl, alkanesulfonyl, sulfamoyl, dialkylsulfamoyl and alkylsulfinyl, etc. The substituent itself may also be optionally substituted. For example, C₁₋₆ acylamide, C₁₋₆ acyloxy, C₁₋₆ alkoxy, aryloxy, alkylthio, C₁₋₆ alkyl, C₁₋₆ acyl, C₆₋₁₀ aryl, C₃₋₈ cycloalkyl, C₂₋₆ alkenyl, C₂₋₆ alkynyl, heterocyclic or heteroaryl groups in the above-mentioned substituents can be optionally substituted by 1-5 groups selected from halogen, C₁₋₆ alkyl, halogenated C₁₋₆ alkyl, C₁₋₆ alkoxy, halogenated C₁₋₆ alkoxy, etc. More preferred substituents include without limitation halogen, hydroxyl, carboxyl, amino, C₁₋₆ amido, C₁₋₆ acyloxy, C₁₋₆ alkoxy, C₁₋₆ alkyl, C₁₋₆ acyl and alkanesulfonyl.

It should be understood that in each embodiment, when the substituent is a heterocyclic group, aryl or heteroaryl, the number thereof is usually 1.

Specifically, the disclosure provides compounds represented by Formula I:

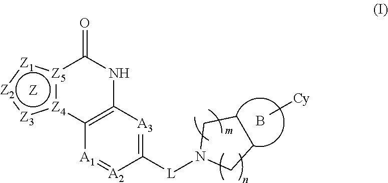

or stereoisomers, tautomers, N-oxides, hydrates, solvates, isotope-substituted derivatives, or pharmaceutically acceptable salts thereof, or mixtures thereof, or prodrugs thereof, wherein:
the Z ring shown as

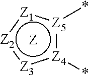

is an optionally substituted 5-membered heteroaryl group, * indicates the position at which the Z ring is attached to the rest of the compound, Z₁, Z₂ and Z₃ are each independently CRI, NR₂, O, N or S, Z₄ and Z₅ are each independently C or N, and Z₄ and Z₅ are not N at the same time;
A₁, A₂ and A₃ are each independently selected from N and CR₃;
L is selected from a bond and an alkylene optionally substituted by R₄ and/or R₅;
the B ring is an optionally substituted heteroaryl or an optionally substituted heterocyclic group;
Cy is selected from a group consisting of an optionally substituted aryl and an optionally substituted heteroaryl;
R₁ is selected from a group consisting of hydrogen, halogen, hydroxyl, cyano, an optionally substituted alkyl, an optionally substituted alkoxy and an optionally substituted carbocyclic group;
R₂ is selected from a group consisting of hydrogen, an optionally substituted alkyl, an optionally substituted alkoxy, an optionally substituted cycloalkyl, an optionally substituted alkenyl and an optionally substituted alkynyl;
R₃ is selected from a group consisting of hydrogen, halogen, an optionally substituted alkyl, an optionally substituted alkoxy and an optionally substituted carbocyclic group;
R₄ and R₅ are each independently selected from a group consisting of halogen, cyano, an optionally substituted alkyl, an optionally substituted alkoxy, an optionally substituted cycloalkyl, an optionally substituted alkenyl, and an optionally substituted alkynyl; or R₄ and R₅ together with the attached C form a ring;
m and n are each independently selected from 1 and 2.

In Formula I and each formula of the disclosure, unless otherwise described, preferably, each alkyl is independently a C₁₋₆ alkyl, preferably a C₁₋₄ alkyl; each alkylene is a C₁₋₆ alkylene, preferably a C₁₋₃ alkylene; each alkenyl is independently a C₂₋₆ alkenyl, preferably C₂₋₄ alkenyl; each alkynyl is independently C₂₋₆ alkynyl, preferably C₂₋₄ alkynyl; each alkoxy is independently C₁₋₆ alkoxy, preferably C₁₋₄ alkoxy. Preferably, when the alkyl, alkenyl, alkynyl and alkoxy are substituted, the substituents can be selected from a group consisting of cyano, hydroxyl, nitro, amino (—NR′R″), aryl, heterocyclic group, heteroaryl, halogen and carboxyl, etc. The number of substituents may be 1-5, R′ and R″ are preferably each independently H, an optionally substituted C₁₋₄ alkyl or an optionally substituted C₃₋₆ cycloalkyl. Preferably, the substituents of the alkyl, alkynyl, alkynyl, and alkoxy groups are the halogen, hydroxyl, or amino groups. For example, the substituted alkyl per se or as a substituent of other groups may be hydroxyalkyl, dihydroxyalkyl, alkylaminoalkyl, dialkylaminoalkyl, heterocyclic alkyl, aralkyl, heteroarylalkyl and haloalkyl, etc. It should be understood, when the substituent is aryl, heteroaryl, heterocyclic group, cyano, nitro and carboxyl, the number thereof is usually 1. When the substituent is halogen, the number of substituents can be up to 5 depending on the carbon chain length of the alkyl, alkenyl, alkynyl and alkoxy groups; exemplary substituents are trifluoromethyl and pentafluoroethyl, etc.

In Formula I and each formula of the disclosure, unless otherwise described, preferably, the number of ring carbon atoms of each carbocyclic group is preferably 3-8. Preferred carbocyclic groups are C₃₋₈ cycloalkyl groups or C₃₋₈ cycloalkenyl. The substituents on the carbocyclic group are preferably C₁₋₄ alkyl, halogenated C₁₋₄ alkyl, C₁₋₄ alkoxy, halogen, hydroxyl, carboxyl, amino (—NR′R″), aryl, heterocyclic group, heteroaryl and carboxyl, etc. The number of substituents may be 1-5, R′ and R″ are preferably each independently H, an optionally substituted C₁₋₄ alkyl or an optionally substituted C₃₋₆ cycloalkyl. It should be understood, when the substituent is aryl, heteroaryl, heterocyclic group, cyano, nitro and carboxyl, the number thereof is usually 1. When the substituent is halogen, the number of substituents can be up to 5.

In Formula I and each formula of the disclosure, unless otherwise described, preferably, the aryl refers to C₆₋₁₄ aryl, the heteroaryl refers to 5-10 membered heteroaryl, and the heterocyclic group refers to 4-10 membered heterocyclic group, such as 4-7 membered monocyclic, or 7-10 membered bicyclic ring, spirocyclic ring or bridged ring. The substituents on each of the aryl, heteroaryl and heterocyclic group can each be independently selected from 1-5 groups consisting of C₁₋₄ alkyl, halogenated C₁₋₄ alkyl, C₁₋₄ alkoxy, halogen, hydroxyl, carboxyl, amino (—NR′R″), an optionally substituted aryl, an optionally substituted heteroaryl, an optionally substituted heterocyclic group, halogen, amido, aminoacyl (—C(O)—NR′R″) and carboxyl, etc.; wherein R′ and R″ each are independently hydrogen, an optionally substituted C₁₋₁₀ alkyl, an optionally substituted C₃₋₈ cycloalkyl, an optionally substituted heterocyclic group, an optionally substituted aryl or an optionally substituted heteroaryl. Preferably, R′ and R″ each are independently hydrogen, an optionally substituted C₁₋₄ alkyl, an optionally substituted C₃₋₆ cycloalkyl or an optionally substituted 3-6 membered heterocyclic group. The number of substituents may be 1-5. The said optionally substituted C₃₋₈ cycloalkyl, optionally substituted aryl, optionally substituted heteroaryl and optionally substituted heterocyclic group may be optionally substituted by 1-5 groups selected from C₁₋₄ alkyl, halogenated C₁₋₄ alkyl, C₁₋₄ alkoxy, halogenated C₁₋₄ alkoxy, halogen, hydroxyl, carboxyl, amino (—NR′R″), and aminoacyl (—C(O)—NR′R″), wherein the said R′ and R″ are preferably each independently H, optionally substituted C₁₋₄ alkyl or optionally substituted C₃₋₆ cycloalkyl. It should be understood, when the substituent is aryl, heteroaryl, heterocyclic group, cyano, nitro or carboxyl, the number thereof is usually 1. When the substituent is halogen, the number of substituents can be up to 5.

In one or more embodiments of the compound of Formula I, the said alkyl, alkoxy, carbocyclic group, cycloalkyl, alkenyl and alkynyl in the definitions of R₁ and R₂ are each optionally substituted by 1 to 5 groups selected from a group consisting of halogen, hydroxyl and —NR′R″, wherein R′ and R″ are each independently H or C₁₋₄ alkyl. Preferably, the said optionally substituted alkyl is an alkyl optionally substituted by 1-5 halogen. In some embodiments, R₁ is preferably hydrogen, halogen or an optionally substituted C₁₋₃ alkyl. In some embodiments, R₂ is preferably hydrogen, an optionally substituted C₁₋₃ alkyl or an optionally substituted C₃₋₆ cycloalkyl, more preferably hydrogen or an optionally substituted C₁₋₃ alkyl.

In one or more embodiments of the compound of Formula I, Z₁ is CR₁, Z₂ and Z₃ are each independently CH or N, Z₄ is N, and Z₅ is C; R₁ is H, halogen, C₁₋₄ alkyl or halogenated C₁₋₄ alkyl.

In one or more embodiments of the compound of Formula I, the Z ring is selected from the following groups:

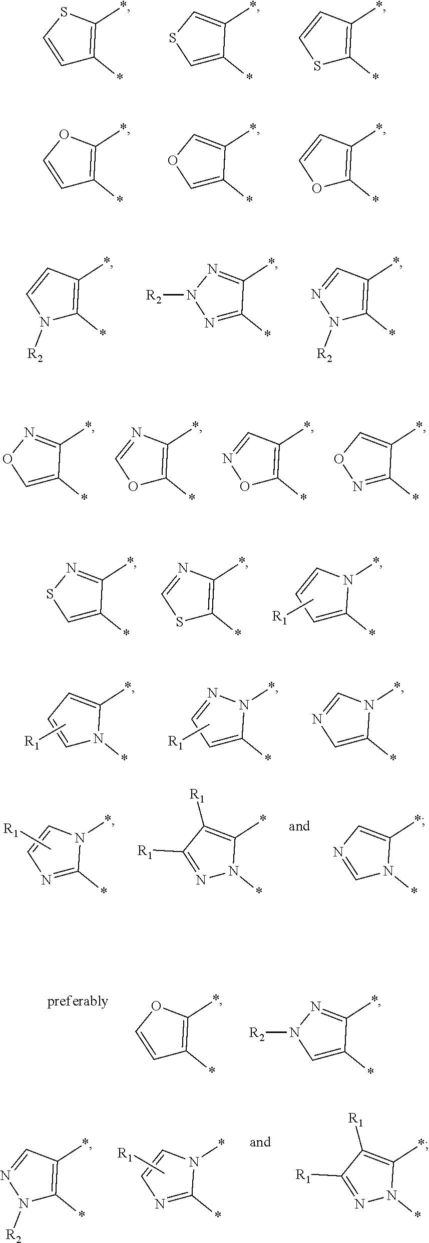

wherein * indicates the position at which the Z ring is attached to the rest of the compound; each R₁ is independently selected from a group consisting of hydrogen, halogen, hydroxyl, cyano, an optionally substituted alkyl, an optionally substituted alkoxy and an optionally substituted carbocyclic group, preferably hydrogen, halogen and an optionally substituted C₁₋₃ alkyl; each R₂ is independently selected from a group consisting of hydrogen, an optionally substituted alkyl, an optionally substituted alkoxy, an optionally substituted cycloalkyl, an optionally substituted alkenyl and an optionally substituted alkynyl, preferably hydrogen, an optionally substituted C₁-3 alkyl and an optionally substituted C₃₋₆ cycloalkyl, more preferably hydrogen or an optionally substituted C₁₋₃ alkyl. Preferably, the said alkyl, alkoxy, carbocyclic group, cycloalkyl, alkenyl and alkynyl are each optionally substituted by 1 to 5 groups selected from halogen, hydroxyl and —NR′R″, wherein R′ and R″ are each independently H or C₁₋₄ alkyl. More preferably, the said optionally substituted alkyl is an alkyl optionally substituted by 1-5 halogen.

In one or more embodiments of the compound of Formula I, A₁, A₂ and A₃ are each independently selected from N and CR₃, wherein R₃ is preferably hydrogen, halogen, an optionally substituted C₁₋₃ alkyl or an optionally substituted C₁₋₃ alkoxy. The said optionally substituted C₁₋₃ alkyl and optionally substituted C₁₋₃ alkoxy can be optionally substituted by 1 to 5 groups selected from halogen, hydroxyl and —NR′R″, wherein R′ and R″ are each independently H or C₁₋₄ alkyl. More preferably, the said optionally substituted alkyl is an alkyl optionally substituted by 1-5 halogen. Preferably, R₃ is hydrogen, halogen or C₁₋₃ alkyl. In one or more embodiments, all of A₁, A₂ and A₃ are CR₃; preferably, R₃ is each independently hydrogen, halogen or C₁₋₃ alkyl. In some embodiments, A₁ is CR₃, both of A₂ and A₃ are CH; or A₁ is CH, A₂ is CR₃, A₃ is CH; or both of A₁ and A₂ are CH, A₃ is CR₃; preferably, R₃ is each independently halogen or C₁₋₃ alkyl. In some preferred embodiments, both of A₁ and A₂ are CH, and A₃ is CR₃; preferably, R₃ is halogen, such as fluoro.

In one or more embodiments of the compound of Formula I, R₄ and R₅ are each independently selected from a group consisting of halogen and an optionally substituted alkyl, preferably, halogen, C₁₋₄ alkyl or halogenated C₁₋₄ alkyl. L is an alkylene group optionally substituted by 1-2 C₁₋₃ alkyl groups, more preferably a C₁₋₃ alkylene group optionally substituted by 1-2 C₁₋₃ alkyl groups, preferably a methylene optionally substituted by 1-2 C₁₋₃ alkyl groups.

In one or more embodiments of the compound of Formula I, L is an unsubstituted alkylene group, more preferably an unsubstituted C₁₋₃ alkylene group, and preferably a methylene group.

In one or more embodiments of the compound of Formula I, B ring is optionally substituted by 1-3 groups selected from a group consisting of halogen, amino, hydroxyl, optionally substituted C₃₋₈ cycloalkyl, optionally substituted C₁₋₆ alkyl. The said optionally substituted C₃₋₈ cycloalkyl can be optionally substituted by 1-3 groups selected from a group consisting of halogen, C₁₋₄ alkyl, C₁₋₄ alkoxy, halogenated C₁₋₄ alkyl, halogenated C₁₋₄ alkoxy, hydroxyl and amino. The said optionally substituted C₁₋₆ alkyl can be optionally substituted by 1-3 groups selected from a group consisting of halogen, hydroxyl and amino.

In one or more embodiments of the compound of Formula I, B ring is an optionally substituted 5- or 6-membered heteroaryl or an optionally substituted 5-6 membered heterocyclic group, preferably containing 1 or 2 heteroatoms selected from O, N and S.

In one or more embodiments of the compound of Formula I, the said

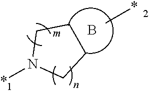

is selected from the following groups:

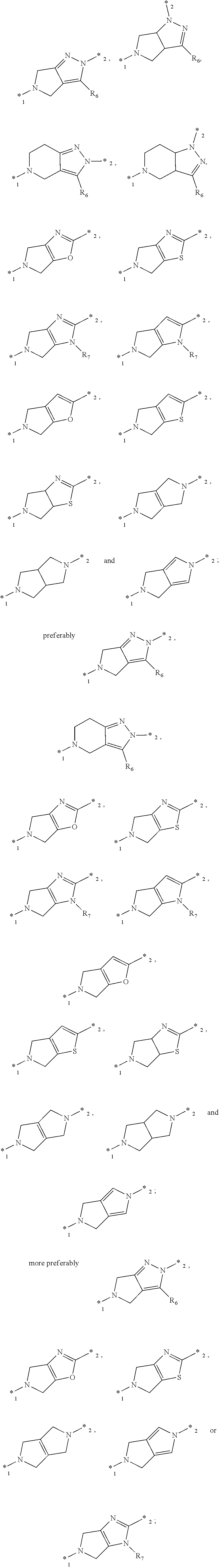

wherein, *1 indicates the position at which B ring is attached to L; *2 indicates the position at which B ring is attached to Cy; R₆ is selected from hydrogen, halogen, amino, hydroxyl and an optionally substituted C₁₋₃ alkyl; R₇ is selected from hydrogen, an optionally substituted C₁₋₃ alkyl and an optionally substituted C₃₋₆ cycloalkyl, preferably hydrogen or optionally substituted C₁₋₃ alkyl. Preferably, the said optionally substituted C₁₋₃ alkyl group is optionally substituted with 1 to 5 groups selected from hydroxyl, halogen and amino.

In one or more embodiments of the compound of Formula I, both of m and n are 1. In one or more embodiments, one of m and n is 2, the other is 1.

In one or more embodiments of the compound of Formula I, Cy is an optionally substituted 6-14 membered aryl or an optionally substituted 5-10 membered heteroaryl, wherein the said optionally substituted 6-14 membered aryl and the optionally substituted 5-10 membered heteroaryl each can be independently substituted by 1-5 substituents selected from a group consisting of halogen, hydroxyl, cyano, an optionally substituted C₁₋₄ alkyl, an optionally substituted C₁₋₄ alkoxy, an optionally substituted C₂₋₄ alkenyl, an optionally substituted C₂₋₄ alkynyl, an optional substituted C₃₋₈ cycloalkyl, amino (—NR′R″), aminoacyl (—C(O)—NR′R″) and carboxyl, wherein the said R′ and R″ each are independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably H, an optionally substituted C₁₋₄ alkyl, an optionally substituted C₃₋₆ cycloalkyl or an optionally substituted 3-6 membered heterocyclic group. Preferably, each of the said optionally substituted C₁₋₄ alkyl, optionally substituted C₁₋₄ alkoxy, optionally substituted C₂₋₄ alkenyl and optionally substituted C₂₋₄ alkynyl can be independently substituted by 1, 2, 3, 4 or 5 substituents selected from a group consisting of halogen, hydroxy, oxygen and amino; the said optionally substituted C₃₋₈ cycloalkyl can be optionally substituted by 1, 2, 3, 4 or 5 substituents selected from a group consisting of halogen, hydroxy, oxygen, C₁₋₄ alkyl and amino; when R′ and R″ are substituted, the substituents can be 1-5 groups selected from halogen, hydroxyl, oxygen, C₁₋₄ alkyl and amino. In some preferred embodiments, the substituent(s) include at least —C(O)—NR′R″, and optionally include one or two of substituents selected from a group consisting of halogen, hydroxyl, cyano, C₁₋₄ alkyl, halogenated C₁₋₄ alkyl and C₃-(cycloalkyl. In some preferred embodiments, Cy is an optionally substituted 5-10 membered heteroaryl, preferably an optionally substituted 5-10 membered nitrogen-containing heteroaryl. Preferably, the 5-10 membered nitrogen-containing heteroaryl is selected from pyrrolyl, pyridyl, pyrimidinyl, pyrazinyl, pyridazinyl, etc. Preferably, the 5-10 membered heteroaryl or 5-10 membered nitrogen-containing heteroaryl is at least substituted by aminoacyl (—C(O)—NR′R″), and optionally further substituted by one or two substituents selected from a group consisting of halogen, hydroxyl, cyano, C₁₋₄ alkyl, halogenated C₁₋₄ alkyl and C₃₋₆ cycloalkyl. Preferably, in the embodiments as described herein, when the said R′ and R″ are substituted, the substituents can be 1-5 groups selected from a group consisting of halogen, hydroxy and amino.

In one or more embodiments of the compound of Formula I, Cy is optionally substituted phenyl, pyridyl, pyrimidinyl, pyrazinyl or pyridazinyl. Preferably, when Cy is substituted, the substituents can be 1-5 groups selected from a group consisting of halogen, hydroxyl, cyano, an optionally substituted C₁₋₄ alkyl, an optionally substituted C₁₋₄ alkoxy, C₃₋₆ cycloalkyl, aminoacyl and carboxyl. More preferably, Cy is substituted by at least one aminoacyl group, preferably, the aminoacyl group is in the para position of Cy. Preferably, the aminoacyl group is —C(O)—NR′R″; wherein the said R′ and R″ each are independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably H, an optionally substituted C₁₋₄ alkyl, an optionally substituted C₃₋₆ cycloalkyl or an optionally substituted 3-6 membered heterocyclic group. Preferably, when R′ and R″ are substituted, the substituents can be 1-5 groups selected from halogen, hydroxyl, oxygen, C₁₋₄ alkyl and amino. In some preferred embodiments, the substituent(s) include at least —C(O)—NR′R″, and optionally include one or two of substituents selected from a group consisting of halogen, hydroxyl, cyano, C₁₋₄ alkyl, halogenated C₁₋₄ alkyl and C₃₋₆ cycloalkyl, further preferably, the aminoacyl group (—C(O)—NR′R″) is located in the para position.

In one or more embodiments of the compound of Formula I, Cy is preferably the following group:

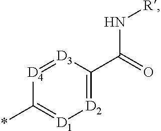

wherein, D₁, D₂, D₃ and D₄ are each independently selected from N and CR₈; R₈ is selected from a group consisting of hydrogen, halogen, hydroxyl, cyano, an optionally substituted C₁₋₄ alkyl, an optionally substituted C₁₋₄ alkoxy, an optionally substituted C₃₋₈ carbocyclic group, amino (—NR′R″), an optionally substituted C₂₋₄ alkenyl and an optionally substituted C₂₋₄ alkynyl. R′ is selected from an optionally substituted C₁₋₁₀ alkyl, an optionally substituted C₃₋₈ cycloalkyl, an optionally substituted heterocyclic group, an optionally substituted aryl or an optionally substituted heteroaryl, preferably an optionally substituted C₁₋₄ alkyl, an optionally substituted C₃₋₆ cycloalkyl or an optionally substituted 3-6 membered heterocyclic group. * indicates the position at which the said group is attached to the B ring. Preferably, the ring containing D₁, D₂, D₃ and D₄ is phenyl, pyridyl, pyrimidinyl, pyrazinyl or pyridazinyl. Preferably, R₈ is H, hydroxyl, halogen, cyano, C₁₋₃ alkyl, C₁₋₃ alkoxy, halogenated C₁₋₃ alkyl or C₃₋₆ cycloalkyl. Preferably, D₃ is N, D₄ is CR₈, both of D₁ and D₂ are CH, wherein R₈ is H, hydroxyl, halogen, cyano, C₁₋₃ alkyl, C₁₋₃ alkoxy, halogenated C₁₋₃ alkyl or C₃₋₆ cycloalkyl. Preferably, when the said R′ is substituted, the substituents can be 1-5 groups selected from a group consisting of halogen, hydroxy and amino. Preferably, R′ is hydrogen, C₁₋₃ alkyl, deuterated C₁₋₃ alkyl, C₃₋₆ cycloalkyl, halogenated C₁₋₃ alkyl, C₁₋₃ alkyl substituted with hydroxyl or 3-6 membered heterocyclic group. Preferably, the 3-6 membered heterocyclic group is an oxygen- and/or nitrogen-containing heterocyclic group, such as oxetanyl, azetidinyl, pyrrolidinyl, piperazinyl, piperidinyl and the like.

In one or more embodiments of the compound of Formula I, the Z ring is selected from the following groups:

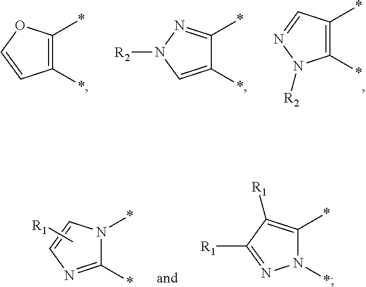

each R₁ is independently hydrogen, halogen, C₁₋₃ alkyl or halo C₁₋₃ alkyl; preferably, in

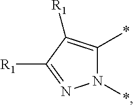

one of R₁ is hydrogen and another R₁ is halogen or C₁₋₃ alkyl; R₂ is C₁₋₃ alkyl;
both of A₁ and A₂ are CH, A₃ is CR₃, wherein R₃ is halogen;
the said

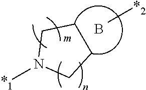

is selected from the following groups:

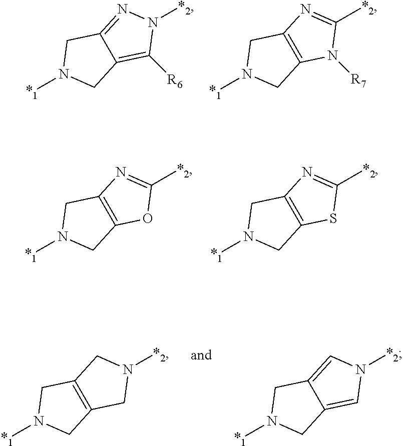

wherein, *1 indicates the position at which B ring is attached to L; *2 indicates the position at which B ring is attached to Cy; R₆ is hydrogen; R₇ is hydrogen;
Cy is:

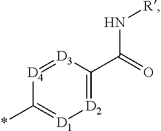

wherein D₃ is N, D₄ is CR₈, both of D₁ and D₂ are CH, wherein R₈ is H, C₁₋₃ alkyl or halogen; R′ is C₁₋₃ alkyl or C₃₋₆ cycloalkyl.

One group of preferred compounds of Formula I in this disclosure is represented by compounds of Formula II (including Formulae IIa, IIb and IIc):

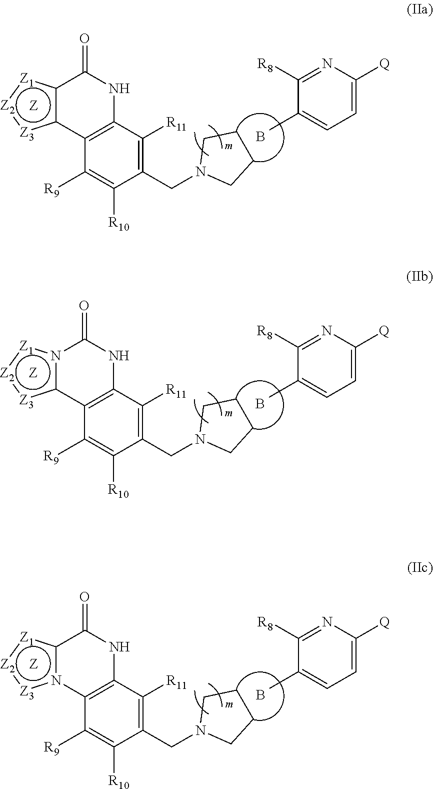

or stereoisomers, tautomers, N-oxides, hydrates, solvates, isotope-substituted derivatives, or pharmaceutically acceptable salts thereof, or mixtures thereof, or prodrugs thereof, wherein:
Z₁, Z₂, Z₃, m, B ring and R₈ are as described in any of the foregoing embodiments;
R₉, R₁₀ and R₁₁ are each independently selected from hydrogen, halogen, an optionally substituted C₁₋₃ alkyl and an optionally substituted C₁₋₃ alkoxy;
Q is cyano, an optionally substituted aminoacyl (—C(O)—NR′R″) or an optionally substituted heteroaryl, wherein R′ and R″ are each independently selected from hydrogen, an optionally substituted C₁₋₁₀ alkyl, an optionally substituted C₃₋₈ cycloalkyl, an optionally substituted heterocyclic group, an optionally substituted aryl and an optionally substituted heteroaryl.

In one or more embodiments of the compound of Formula II (including Formulae IIa, IIb and IIc), the Z ring is selected from the following groups:

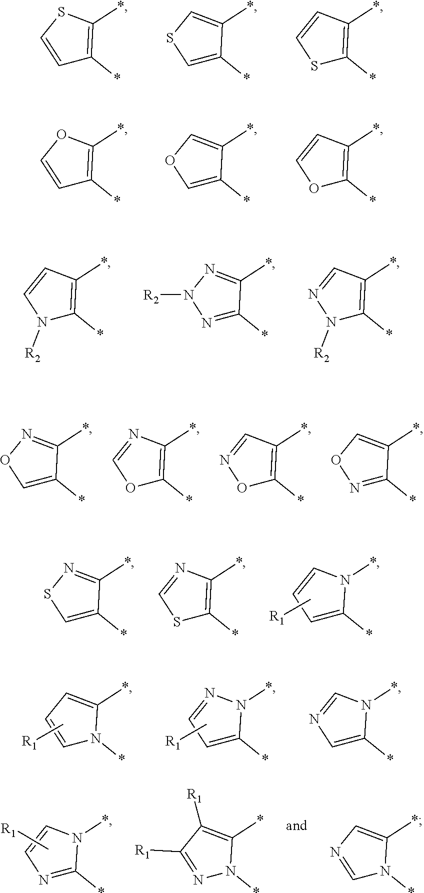

preferably

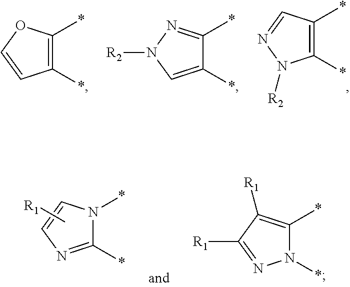

wherein * indicates the position at which the Z ring is attached to the rest of the compound; R₁ and R₂ are as described in any of the foregoing embodiments.

In one or more embodiments of the compound of Formula II (including Formulae IIa, IIb and IIc), when R₉, R₁₀ and R₁₁ are substituted, the substituent can be 1-5 groups selected from a group consisting of halogen, hydroxyl and —NR′R″, wherein R′ and R″ are each independently H or C₁₋₄ alkyl. In some embodiments, R₉, R₁₀ and R₁₁ are each independently hydrogen, halogen, an optionally substituted C₁₋₃ alkyl or an optionally substituted C₁₋₃ alkoxy; preferably hydrogen, halogen or C₁₋₃ alkyl; preferably, at least one of R₉, R₁₀ and R₁₁ is a non-hydrogen substituent, preferably the non-hydrogen substituent is halogen. In one or more embodiments, R₉ is halogen or C₁₋₃ alkyl, both of R₁₀ and R₁₁ are H; or R₉ is H, R₁₀ is halogen or C₁₋₃ alkyl, R₁ is H; or both of R₉ and R₁₀ are H, R₁₁ is halogen or C₁₋₃ alkyl. In some preferred embodiments, both of R₉ and R₁₀ are H, R₁₁ is halogen, such as fluoro.

In one or more embodiments of the compound of Formula II (including Formulae IIa, IIb and IIc), B ring is optionally substituted by 1-3 groups selected from a group consisting of halogen, amino, hydroxyl, optionally substituted C₃₋₈ cycloalkyl, optionally substituted C₁₋₆ alkyl. The said optionally substituted C₃₋₈ cycloalkyl can be optionally substituted by 1-3 groups selected from a group consisting of halogen, C₁₋₄ alkyl, C₁₋₄ alkoxy, halogenated C₁₋₄ alkyl, halogenated C₁₋₄ alkoxy, hydroxyl and amino. The said optionally substituted C₁₋₆ alkyl can be optionally substituted by 1-3 groups selected from a group consisting of halogen, hydroxyl and amino.

In one or more embodiments of the compound of Formula II (including Formulae IIa, IIb and IIc), B ring is an optionally substituted 5- or 6-membered heteroaryl or an optionally substituted 5-6 membered heterocyclic group, preferably containing 1 or 2 heteroatoms selected from O, N and S.

In one or more embodiments of the compound of Formula II (including Formulae IIa, IIb and IIc),

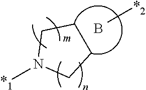

is selected from the following groups:

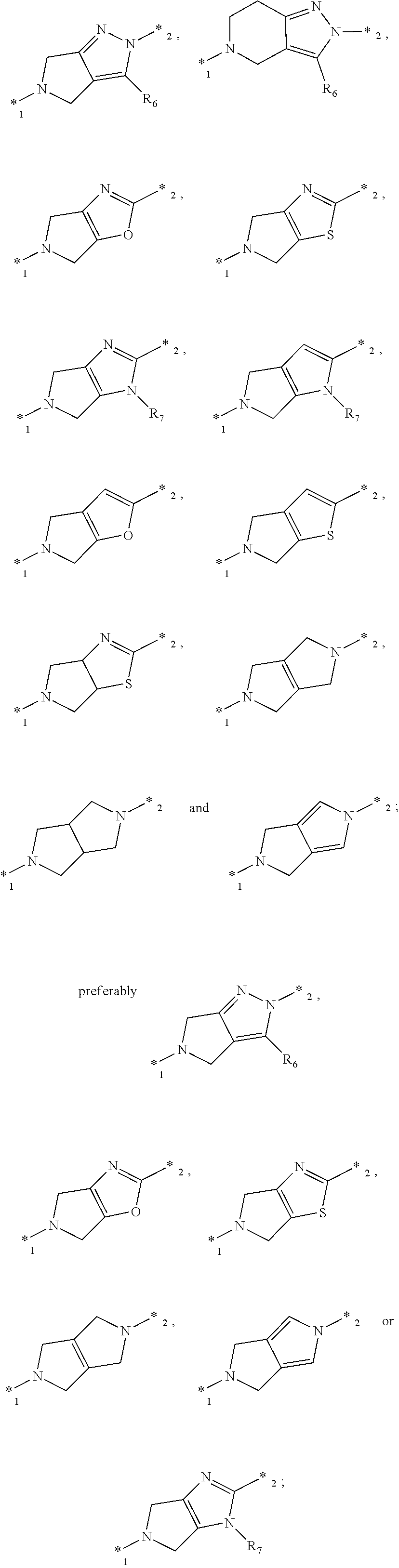

wherein, *1 indicates the position at which B ring is attached to methylene; *2 indicates the position at which B ring is attached to pyridyl; R₆ is selected from hydrogen, halogen, amino, hydroxyl and an optionally substituted C₁₋₃ alkyl; R₇ is selected from hydrogen, an optionally substituted C₁₋₃ alkyl and an optionally substituted C₃₋₆ cycloalkyl, preferably hydrogen or optionally substituted C₁₋₃ alkyl. Preferably, the optionally substituted C₁₋₃ alkyl group is optionally substituted with 1 to 5 groups selected from hydroxyl, halogen and amino.

In one or more embodiments of the compound of Formula II (including Formulae IIa, IIb and IIc), m is 1 or 2, preferably 1.

In one or more embodiments of the compound of Formula II (including Formulae IIa, IIb and IIc), R₈ is hydrogen, hydroxyl, halogen, cyano, an optionally substituted C₁₋₃ alkyl, an optionally substituted C₁₋₃ alkoxy or an optionally substituted C₃₋₆ cycloalkyl; preferably hydrogen, hydroxyl, halogen, C₁₋₃ alkyl, halogenated C₁₋₃ alkyl or C₃₋₆ cycloalkyl, more preferably hydrogen, hydroxyl, halogen or C₁₋₃ alkyl.

In one or more embodiments of the compound of Formula II (including Formulae IIa, IIb and IIc), Q is —C(O)—NR′R″ as described in any of the foregoing embodiments, wherein the said R′ and R″ each are independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably H, an optionally substituted C₁₋₄ alkyl, an optionally substituted C₃₋₆ cycloalkyl or an optionally substituted 3-6 membered heterocyclic group. In one or more embodiments, Q is an optionally substituted 5-membered heteroaryl can be optionally substituted by 1-3 substituents selected from a group consisting of halogen, cyano, C₁₋₃ alkyl, halogenated C₁₋₃ alkyl, C₁₋₃ alkoxy and halogenated C₁₋₃ alkoxy, the said 5-membered heteroaryl is preferably pyrrolyl, pyrazolyl, imidazolyl or triazolyl.

One group of preferred compounds of Formula I in this disclosure is represented by compounds of Formula III:

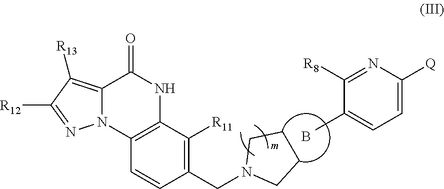

or stereoisomers, tautomers, N-oxides, hydrates, solvates, isotope-substituted derivatives, or pharmaceutically acceptable salts thereof, or mixtures thereof, or prodrugs thereof, wherein:
R₈, R₁₁, m, B ring and Q are as described in any of the foregoing embodiments;
R₁₂ and R₁₃ are each independently selected from hydrogen, halogen, cyano, an optionally substituted C₁₋₃ alkyl and an optionally substituted C₁₋₃ alkoxy.

In one or more embodiments of the compound of Formula III, R₁₂ and R₁₃ are each independently hydrogen, halogen, C₁₋₃ alkyl or halogenated C₁₋₃ alkyl. In one or more embodiments, R₁₂ is hydrogen, R₁₃ is halogen, C₁₋₃ alkyl or halogenated C₁₋₃ alkyl. In one or more embodiments, R₁₂ is halogen, C₁₋₃ alkyl or halogenated C₁₋₃ alkyl, R₁₃ is hydrogen.

In one or more embodiments of the compound of Formula III, R₁₁ is hydrogen, halogen, C₁₋₃ alkyl or C₁₋₃ alkoxy. Preferably, R₁₁ is hydrogen, halogen or C₁₋₃ alkyl. More preferably, R₁₁ is halogen, such as fluoro.

In one or more embodiments of the compound of Formula III, B ring is optionally substituted by 1-3 groups selected from a group consisting of halogen, amino, hydroxyl, optionally substituted C₃₋₈ cycloalkyl, optionally substituted C₁₋₆ alkyl. The said optionally substituted C₃₋₈ cycloalkyl can be optionally substituted by 1-3 groups selected from a group consisting of halogen, C₁₋₄ alkyl, C₁₋₄ alkoxy, halogenated C₁₋₄ alkyl, halogenated C₁₋₄ alkoxy, hydroxyl and amino. The said optionally substituted C₁₋₆ alkyl can be optionally substituted by 1-3 groups selected from a group consisting of halogen, hydroxyl and amino.

In one or more embodiments of the compound of Formula III, B ring is an optionally substituted 5- or 6-membered heteroaryl or an optionally substituted 5-6 membered heterocyclic group, preferably containing 1 or 2 heteroatoms selected from O, N and S.

In one or more embodiments of the compound of Formula III,

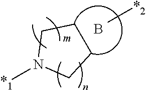

is selected from the following groups:

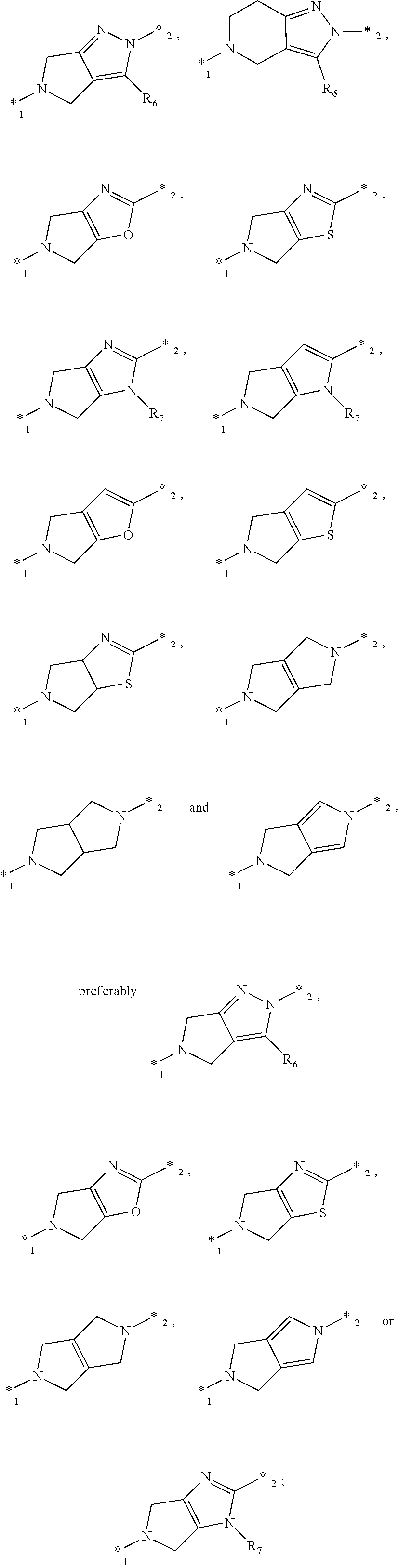

wherein, *1 indicates the position at which pyrrole ring is attached to methylene; *2 indicates the position at which B ring is attached to pyridyl; R₆ is selected from hydrogen, halogen, amino, hydroxyl and an optionally substituted C₁₋₃ alkyl; R₇ is selected from hydrogen, an optionally substituted C₁₋₃ alkyl and an optionally substituted C₃₋₆ cycloalkyl, preferably hydrogen or optionally substituted C₁₋₃ alkyl.

In one or more embodiments of the compound of Formula III, m is 1 or 2, preferably 1.

In one or more embodiments of the compound of Formula III, R₈ is hydrogen, hydroxyl, halogen, cyano, an optionally substituted C₁₋₃ alkyl, an optionally substituted C₁₋₃ alkoxy or an optionally substituted C₃₋₆ cycloalkyl; preferably hydrogen, hydroxyl, halogen, C₁₋₃ alkyl, halogenated C₁₋₃ alkyl or C₃₋₆ cycloalkyl, more preferably hydrogen, hydroxyl, halogen or C₁₋₃ alkyl.

In one or more embodiments of the compound of Formula III, Q is —C(O)—NR′R″ as described in any of the foregoing embodiments, wherein the said R′ and R″ each are independently H, an optionally substituted C₁₋₁₀ alkyl, an optionally substituted C₃₋₈ cycloalkyl, an optionally substituted heterocyclic group, an optionally substituted aryl or an optionally substituted heteroaryl; preferably H, an optionally substituted C₁₋₄ alkyl, an optionally substituted C₃₋₆ cycloalkyl or an optionally substituted 3-6 membered heterocyclic group. In one or more embodiments, Q is an optionally substituted 5-membered heteroaryl can be optionally substituted by 1-3 substituents selected from a group consisting of halogen, cyano, C₁₋₃ alkyl, halogenated C₁₋₃ alkyl, C₁₋₃ alkoxy and halogenated C₁₋₃ alkoxy, the said 5-membered heteroaryl is preferably pyrrolyl, pyrazolyl, imidazolyl or triazolyl.

It should be understood that although Z₁, Z₂, Z₃, Z₄, Z₅, A₁, A₂, A₃, L, m, n, B ring, Cy, R₈, R₉, R₁₀, R₁₁, R₁₂, R₁₃ and Q in Formula I (including Formulae II and III) are described separately above, the described features, especially the preferred features, can be arbitrarily combined to form the scope of different compounds of Formula I (including Formulae II and III) in this disclosure. For example, in some embodiments of compounds of Formula I (including Formulae II and III) of this disclosure.

The preferred compounds of this disclosure, without limitation:

| | |
|---|---|
| 1-1 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-2(4H)-yl)-N-methylpicolinamide |
| 1-2 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-1(4H)-yl)-N-methylpicolinamide |
| 2 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-2(4H)-yl)-N,6-dimethylpicolinamide |
| 3 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-2(4H)-yl)-6-fluoro-N-methylpicolinamide |
| 4 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-2(4H)-yl)-6-chloro-N-methylpicolinamide |
| 5 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-2(4H)-yl)-6-hydroxy-N-methylpicolinamide |
| 6 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-2(4H)-yl)-N-ethylpicolinamide |
| 7 | N-ethyl-6-fluoro-5-(5-((6-fluoro-3-methyl-4-oxo-4,5-dihydropyrazolo[1,5- |
| | a]quinoxalin-7-yl)methyl)-5,6-dihydropyrrolo[3,4-c]pyrazol-2(4H)-yl)picolinamide |
| 8 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrazol-2(4H)-yl)-N-cyclopropylpicolinamide |
| 9 | N-cyclopropyl-6-fluoro-5-(5-((6-fluoro-3-methyl-4-oxo-4,5-dihydropyrazolo[1,5- |
| | a]quinoxalin-7-yl)methyl)-5,6-dihydropyrrolo[3,4-c]pyrazol-2(4H)-yl)picolinamide |
| 10-1 | 5-(5-((6-fluoro-3-methyl-4-oxo-4,5-dihydropyrazolo[1,5-a]quinoxalin-7-yl)methyl)- |
| | 4,5,6,7-tetrahydro-2H-pyrazolo[4,3-c]pyridin-2-yl)-N-methylpicolinamide |
| 10-2 | 5-(5-((6-fluoro-3-methyl-4-oxo-4,5-dihydropyrazolo[1,5-a]quinoxalin-7-yl)methyl)- |
| | 4,5,6,7-tetrahydro-1H-pyrazolo[4,3-c]pyridin-1-yl)-N-methylpicolinamide |
| 11 | 5-(5-((6-fluoro-3-methyl-4-oxo-4,5-dihydropyrazolo[1,5-a]quinoxalin-7-yl)methyl)- |
| | 4,5,6,7-tetrahydro-2H-pyrazolo[4,3-c]pyridin-2-yl)-N,6-dimethylpicolinamide |
| 12 | 5-(5-((6-fluoro-3-methyl-4-oxo-4,5-dihydropyrazolo[1,5-a]quinoxalin-7-yl)methyl)- |
| | 4,5,6,7-tetrahydro-2H-pyrazolo[4,3-c]pyridin-2-yl)-6-fluoro-N-methylpicolinamide |
| 13 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-N-methylpicolinamide |
| 14 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-N,6-dimethylpicolinamide |
| 15 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-6-fluoro-N-methylpicolinamide |
| 16 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-6-chloro-N-methylpicolinamide |
| 17 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-6-hydroxy-N-methylpicolinamide |
| 18 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-N-ethylpicolinamide |
| 19 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-N-ethyl-6-fluoropicolinamide |
| 20 | 5-(5-((3,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-N-cyclopropylpicolinamide |
| 21 | N-cyclopropyl-5-(5-((3,6-difluoro-4-oxo-4,5-dihydropyrazolo[1,5-a]quinoxalin-7- |
| | yl)methyl)-5,6-dihydropyrrolo[3,4-c]pyrazol-2(4H)-yl)-6-fluoropicolinamide |
| 22 | 5-(5-((3,6-difluoro-4-oxo-4,5-dihydropyrazolo[1,5-a]quinoxalin-7-yl)methyl)- |
| | 4,5,6,7-tetrahydro-2H-pyrazolo[4,3-c]pyridin-2-yl)-N-methylpicolinamide |
| 23 | 5-(5-((3,6-difluoro-4-oxo-4,5-dihydropyrazolo[1,5-a]quinoxalin-7-yl)methyl)- |
| | 4,5,6,7-tetrahydro-2H-pyrazolo[4,3-c]pyridin-2-yl)-N,6-dimethylpicolinamide |
| 24 | 5-(5-((3,6-difluoro-4-oxo-4,5-dihydropyrazolo[1,5-a]quinoxalin-7-yl)methyl)- |
| | 4,5,6,7-tetrahydro-2H-pyrazolo[4,3-c]pyridin-2-yl)-6-fluoro-N-methylpicolinamide |
| 25 | 5-(5-((6-fluoro-4-oxo-3-(trifluoromethyl)-4,5-dihydropyrazolo[1,5-a]quinoxalin-7- |
| | yl)methyl)-5,6-dihydropyrrolo[3,4-c]pyrazol-2(4H)-yl)-N-methylpicolinamide |
| 26 | 5-(5-((6-fluoro-4-oxo-3-(trifluoromethyl)-4,5-dihydropyrazolo[1,5-a]quinoxalin-7- |
| | yl)methyl)-5,6-dihydropyrrolo[3,4-c]pyrazol-2(4H)-yl)-6-fluoro-N-methylpicolinamide |
| 27 | 5-(5-((2,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-N-methylpicolinamide |
| 28 | 5-(5-((6-fluoro-4-oxo-4,5-dihydrofuro[2,3-c]quinolin-7-yl)methyl)-5,6- |
| | dihydropyrrolo[3,4-c]pyrazol-2(4H)-yl)-N-methylpicolinamide |
| 29 | 5-(5-((6-fluoro-2-methyl-4-oxo-4,5-dihydro-2H-pyrazolo[3,4-c]quinolin-7- |
| | yl)methyl)-5,6-dihydropyrrolo[3,4-c]pyrazol-2(4H)-yl)-N-methylpicolinamide |
| 30 | 5-(5-((6-fluoro-1-methyl-4-oxo-4,5-dihydro-1H-pyrazolo[4,3-c]quinolin-7- |
| | yl)methyl)-5,6-dihydropyrrolo[3,4-c]pyrazol-2(4H)-yl)-N-methylpicolinamide |
| 31 | 5-(5-((6-fluoro-3-methyl-4-oxo-4,5-dihydropyrazolo[1,5-a]quinoxalin-7-yl)methyl)- |
| | 3-methyl-5,6-dihydropyrrolo[3,4-c]pyrazol-2(4H)-yl)-N-methylpicolinamide |
| 32 | 5-(5-((6-fluoro-3-methyl-4-oxo-4,5-dihydropyrazolo[1,5-a]quinoxalin-7-yl)methyl)- |
| | 5,6-dihydro-4H-pyrrolo[3,4-d]oxazol-2-yl)-N-methylpicolinamide |
| 33 | 5-(5-((6-fluoro-3-methyl-4-oxo-4,5-dihydropyrazolo[1,5-a]quinoxalin-7-yl)methyl)- |
| | 5,6-dihydro-4H-pyrrolo[3,4-d]thiazol-2-yl)-N-methylpicolinamide |
| 34 | 5-(5-((6-fluoro-3-methyl-4-oxo-4,5-dihydropyrazolo[1,5-a]quinoxalin-7-yl)methyl)- |
| | 1,4,5,6-tetrahydropyrrolo[3,4-d]imidazol-2-yl)-N-methylpicolinamide |
| 35 | 5-(5-((6-fluoro-3-methyl-4-oxo-4,5-dihydropyrazolo[1,5-a]quinoxalin-7-yl)methyl)- |
| | 1-methyl-1,4,5,6-tetrahydropyrrolo[3,4-d]imidazol-2-yl)-N-methylpicolinamide |
| 36 | 5-(5-((6-fluoro-3-methyl-4-oxo-4,5-dihydropyrazolo[1,5-a]quinoxalin-7-yl)methyl)- |
| | 1-methyl-1,4,5,6-tetrahydropyrrolo[3,4-b]pyrrol-2-yl)-N-methylpicolinamide |
| 37 | 5-(5-((6-fluoro-3-methyl-4-oxo-4,5-dihydropyrazolo[1,5-a]quinoxalin-7-yl)methyl)- |
| | 5,6-dihydro-4H-furo[2,3-c]pyrrol-2-yl)-N-methylpicolinamide |
| 38 | 5-(5-((6-fluoro-3-methyl-4-oxo-4,5-dihydropyrazolo[1,5-a]quinoxalin-7-yl)methyl)- |
| | 5,6-dihydro-4H-thieno[2,3-c]pyrrol-2-yl)-N-methylpicolinamide |
| 39 | 5-(5-((6-fluoro-3-methyl-4-oxo-4,5-dihydropyrazolo[1,5-a]quinoxalin-7-yl)methyl)- |
| | 5,6-dihydropyrrolo[3,4-c]pyrrol-2(4H)-yl)-N-methylpicolinamide |
| 40 | 5-(5-((3,6-difluoro-4-oxo-4,5-dihydropyrazolo[1,5-a]quinoxalin-7-yl)methyl)-3- |
| | methyl-5,6-dihydropyrrolo[3,4-c]pyrazol-2(4H)-yl)-N-methylpicolinamide |
| 41 | 5-(5-((3,6-difluoro-4-oxo-4,5-dihydropyrazolo[1,5-a]quinoxalin-7-yl)methyl)-5,6- |
| | dihydro-4H-pyrrolo[3,4-d]oxazol-2-yl)-N-methylpicolinamide |
| 42 | 5-(5-((3,6-difluoro-4-oxo-4,5-dihydropyrazolo[1,5-a]quinoxalin-7-yl)methyl)-5,6- |
| | dihydro-4H-pyrrolo[3,4-d]thiazol-2-yl)-N-methylpicolinamide |
| 43 | 5-(5-((3,6-difluoro-4-oxo-4,5-dihydropyrazolo[1,5-a]quinoxalin-7-yl)methyl)- |
| | 1,4,5,6-tetrahydropyrrolo[3,4-d]imidazol-2-yl)-N-methylpicolinamide |
| 44 | 5-(5-((3,6-difluoro-4-oxo-4,5-dihydropyrazolo[1,5-a]quinoxalin-7-yl)methyl)-1- |
| | methyl-1,4,5,6-tetrahydropyrrolo[3,4-d]imidazol-2-yl)-N-methylpicolinamide |
| 45 | 5-(5-((3,6-difluoro-4-oxo-4,5-dihydropyrazolo[1,5-a]quinoxalin-7-yl)methyl)-1- |
| | methyl-1,4,5,6-tetrahydropyrrolo[3,4-b]pyrrol-2-yl)-N-methylpicolinamide |
| 46 | 5-(5-((3,6-difluoro-4-oxo-4,5-dihydropyrazolo[1,5-a]quinoxalin-7-yl)methyl)-5,6- |
| | dihydro-4H-furo[2,3-c]pyrrol-2-yl)-N-methylpicolinamide |
| 47 | 5-(5-((3,6-difluoro-4-oxo-4,5-dihydropyrazolo[1,5-a]quinoxalin-7-yl)methyl)-5,6- |
| | dihydro-4H-thieno[2,3-c]pyrrol-2-yl)-N-methylpicolinamide |
| 48 | 5-(5-((3,6-difluoro-4-oxo-4,5-dihydropyrazolo[1,5-a]quinoxalin-7-yl)methyl)-5,6- |
| | dihydropyrrolo[3,4-c]pyrrol-2(4H)-yl)-N-methylpicolinamide |
| 49 | 5-(5-((7-fluoro-2-methyl-5-oxo-5,6-dihydroimidazo[1,2-c]quinazolin-8-yl)methyl)- |
| | 5,6-dihydropyrrolo[3,4-c]pyrazol-2(4H)-yl)-N-methylpicolinamide |
| 50 | N-ethyl-5-(5-((7-fluoro-2-methyl-5-oxo-5,6-dihydroimidazo[1,2-c]quinazolin-8- |
| | yl)methyl)-5,6-dihydropyrrolo[3,4-c]pyrazol-2(4H)-yl)picolinamide |
| 51 | 6-fluoro-5-(5-((7-fluoro-2-methyl-5-oxo-5,6-dihydroimidazo[1,2-c]quinazolin-8- |
| | yl)methyl)-5,6-dihydropyrrolo[3,4-c]pyrazol-2(4H)-yl)-N-methylpicolinamide |
| 52 | N-ethyl-6-fluoro-5-(5-((7-fluoro-2-methyl-5-oxo-5,6-dihydroimidazo[1,2- |
| | c]quinazolin-8-yl)methyl)-5,6-dihydropyrrolo[3,4-c]pyrazol-2(4H)-yl)picolinamide |
| 53 | N-ethyl-5-(5-((6-fluoro-4-oxo-4,5-dihydrofuro[2,3-c]quinolin-7-yl)methyl)-5,6- |
| | dihydropyrrolo[3,4-c]pyrazol-2(4H)-yl)picolinamide |
| 54 | 6-fluoro-5-(5-((6-fluoro-4-oxo-4,5-dihydrofuro[2,3-c]quinolin-7-yl)methyl)-5,6- |
| | dihydropyrrolo[3,4-c]pyrazol-2(4H)-yl)-N-methylpicolinamide |
| 55 | N-ethyl-6-fluoro-5-(5-((6-fluoro-4-oxo-4,5-dihydrofuro[2,3-c]quinolin-7- |
| | yl)methyl)-5,6-dihydropyrrolo[3,4-c]pyrazol-2(4H)-yl)picolinamide |
| 56 | 5-(5-((2,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-6-fluoro-N-methylpicolinamide |
| 57 | 5-(5-((2,6-difluoro-4-oxo-4,5-dihydropyrazolo[1,5-a]quinoxalin-7-yl)methyl)-5,6- |
| | dihydropyrrolo[3,4-c]pyrazol-2(4H)-yl)-N-ethyl-6-fluoropicolinamide |
| 58 | (R)-3-((3,6-difluoro-4-oxo-4,5-dihydropyrazolo[1,5-a]quinoxalin-7-yl)methyl)-N- |
| | methyl-1,2,3,4,4a,5-hexahydro-7H-pyrazino[2,1-c]pyrido[3,2-e][1,4]oxazepine-9- |
| | carboxamide |
| 59 | 5-(5-((3,6-difluoro-4-oxo-4,5-dihydropyrazolo[1,5-a]quinoxalin-7-yl)methyl)- |
| | 3,4,5,6-tetrahydropyrrolo[3,4-c]pyrrol-2(1H)-yl)-6-fluoro-N-methylpicolinamide |
| 60 | 5-(5-((3,6-difluoro-4-oxo-4,5-dihydropyrazolo[1,5-a]quinoxalin-7-yl)methyl)- |
| | 3a,5,6,6a-tetrahydro-4H-pyrrolo[3,4-d]thiazol-2-yl)-N-methylpicolinamide |
| 61 | 5-(5-((3,6-difluoro-4-oxo-4,5-dihydropyrazolo[1,5-a]quinoxalin-7- |
| | yl)methyl)hexahydropyrrolo[3,4-c]pyrrol-2(1H)-yl)-6-fluoro-N-methylpicolinamide |
| 62 | 5-(5-((3,6-difluoro-4-oxo-4,5-dihydropyrazolo[1,5-a]quinoxalin-7-yl)methyl)-5,6- |
| | dihydropyrrolo[3,4-c]pyrrol-2(4H)-yl)-6-fluoro-N-methylpicolinamide |
| 63 | 6-fluoro-5-(5-((6-fluoro-3-methyl-4-oxo-4,5-dihydropyrazolo[1,5-a]quinoxalin-7- |
| | yl)methyl)-5,6-dihydropyrrolo[3,4-c]pyrrol-2(4H)-yl)-N-methylpicolinamide |
| 64 | 6-fluoro-5-(5-((6-fluoro-3-methyl-4-oxo-4,5-dihydropyrazolo[1,5-a]quinoxalin-7- |
| | yl)methyl)hexahydropyrrolo[3,4-c]pyrrol-2(1H)-yl)-N-methylpicolinamide |
| 65 | 5-(5-((3,6-difluoro-4-oxo-4,5-dihydropyrazolo[1,5-a]quinoxalin-7-yl)methyl)-5,6- |
| | dihydro-4H-pyrrolo[3,4-d]oxazol-2-yl)-6-fluoro-N-methylpicolinamide |
| 66 | 5-(5-((3,6-difluoro-4-oxo-4,5-dihydropyrazolo[1,5-a]quinoxalin-7-yl)methyl)-5,6- |
| | dihydro-4H-furo[2,3-c]pyrrol-2-yl)-6-fluoro-N-methylpicolinamide |

or stereoisomers, tautomers, N-oxides, hydrates, isotope-substituted derivatives, solvates or pharmaceutically acceptable salts thereof, or mixtures thereof, or prodrugs thereof.

Some of the compounds of the present disclosure may exist as stereoisomers including optical isomers. The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁₋₄ alcohol according to methods known in the art); esters of hydroxy containing compounds (e.g., those obtained by condensation with a C₁₋₄ carboxylic acid, C₃₋₆ diacid or anhydride thereof, such as succinic anhydride and fumaric anhydride according to methods known in the art); imines of amino containing compounds (e.g., those obtained by condensation with a C₁₋₄ aldehyde or ketone according to methods known in the art); carbamate of amino containing compounds, such as those described by Leu, et al., (J. Med. Chem. 42: 3623-3628 (1999)) and Greenwald, et al., (J. Med. Chem. 42: 3657-3667 (1999)); and acetals and ketals of alcohol-containing compounds (e.g., those obtained by condensation with chloromethyl methyl ether or chloromethyl ethyl ether according to methods known in the art).

The compounds of this disclosure may be prepared using methods known to those skilled in the art, or the novel methods of this disclosure. Specifically, the compounds of this disclosure with Formula I (including Formulae II and III) can be prepared as illustrated by the exemplary reaction in Scheme 1. Reaction of 4-bromo-3-fluoro-2-nitroaniline, NaNO₂, SnCl₂·2H₂O and hydrochloric acid produced (4-bromo-3-fluoro-2-nitrophenyl)hydrazine. Reaction of (4-bromo-3-fluoro-2-nitrophenyl)hydrazine and methyl-4-(dimethylamino)-3-methyl-2-oxobut-3-enoate under the catalysis of acetic acid produced methyl 1-(4-bromo-3-fluoro-2-nitrophenyl)-4-methyl-1H-pyrazole-5-carboxylate. Reaction of methyl 1-(4-bromo-3-fluoro-2-nitrophenyl)-4-methyl-1H-pyrazole-5-carboxylate, Fe and acetic acid produced 7-bromo-6-fluoro-3-methylpyrazolo[1,5-a]quinoxalin-4(5H)-one. Reaction of 7-bromo-6-fluoro-3-methylpyrazolo[1,5-a]quinoxalin-4(5H)-one and (tributylstannyl)methanol (Bu₃SnCH₂OH) under the catalysis of chloro(2-dicyclohexylphosphino-2′,4′,6′-tri-1-propyl-1,1′-biphenyl)(2′ -amino-1,1′-biphenyl-2-yl) palladium(II) (Xphos Pd G2) produced 6-fluoro-7-(hydroxymethyl)-3-methylpyrazolo[1,5-a]quinoxalin-4(5H)-one. Reaction of 6-fluoro-7-(hydroxymethyl)-3-methylpyrazolo[1,5-a]quinoxalin-4(5H)-one and HBr produced 7-(bromomethyl)-6-fluoro-3-methylpyrazolo[1,5-a]quinoxalin-4(5H)-one. Reaction of 7-(bromomethyl)-6-fluoro-3-methylpyrazolo[1,5-a]quinoxalin-4(5H)-one and 5-(5,6-dihydropyrrolo[3,4-c]pyrazol-2(4H)-yl)-N-methylpicolinamide under the catalysis of DIEA and KI produced the target compound 5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide.

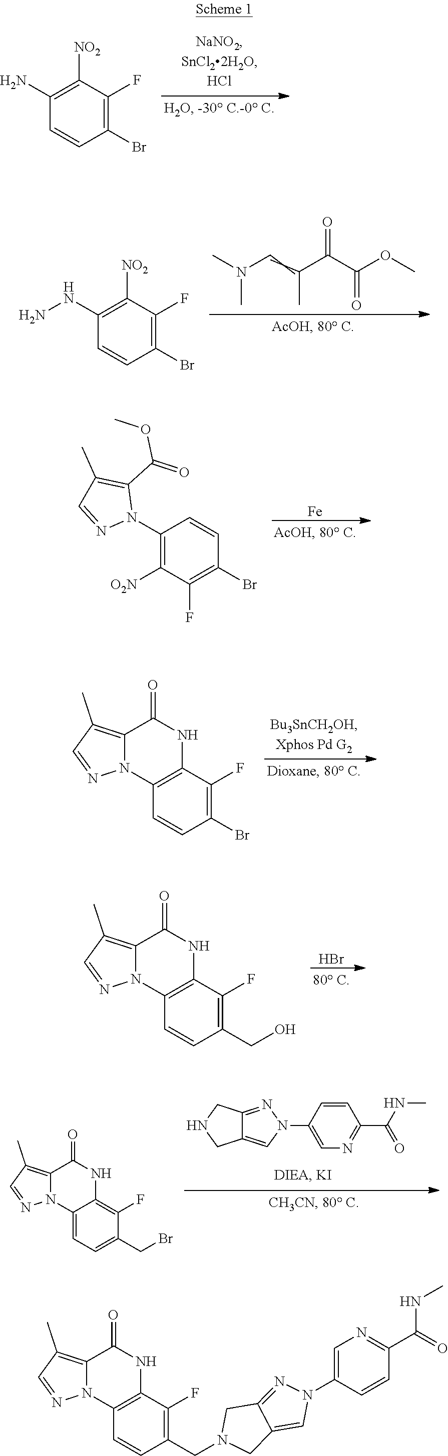

Other related compounds can be prepared using similar methods. For example, replacement of 5-(5,6-dihydropyrrolo[3,4-c]pyrazol-2(4H)-yl)-N-methylpicolinamide with 5-(5,6-dihydropyrrolo[3,4-c]pyrazol-2(4H)-yl)-N,6-dimethylpicolinamide produced the target compound 5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N,6-dimethylpicolinamide. Replacement of 5-(5,6-dihydropyrrolo[3,4-c]pyrazol-2(4H)-yl)-N-methylpicolinamide with 5-(5,6-dihydropyrrolo[3,4-c]pyrazol-2(4H)-yl)-6-fluoro-N-methylpicolinamide produced the target compound 5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-6-fluoro-N-methylpicolinamide. Replacement of 5-(5,6-dihydropyrrolo[3,4-c]pyrazol-2(4H)-yl)-N-methylpicolinamide with 6-chloro-5-(5,6-dihydropyrrolo[3,4-c]pyrazol-2(4H)-yl)-N-methylpicolinamide produced the target compound 5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-6-chloro-N-methylpicolinamide.

The compounds of this disclosure may be prepared using methods known to those skilled in the art, or the novel methods of this disclosure. Specifically, the compounds of this disclosure with Formula I (including Formulae II and III) can be prepared as illustrated by the exemplary reaction in Scheme 2. Reaction of 3-bromo-2,6-difluoroaniline and 4-fluoro-1H-pyrazole-5-carbonyl chloride under the catalysis of LiHMDS produced N-(3-bromo-2,6-difluorophenyl)-4-fluoro-1H-pyrazole-5-carboxamide. Ring closure of N-(3-bromo-2,6-difluorophenyl)-4-fluoro-1H-pyrazole-5-carboxamide under basic conditions produced 7-bromo-3,6-difluoropyrazolo[1,5-a]quinoxalin-4(5H)-one. Reaction of 7-bromo-3,6-difluoropyrazolo[1,5-a]quinoxalin-4(5H)-one and Bu₃SnCH₂OH under the catalysis ofXphos Pd G2 produced 3,6-difluoro-7-(hydroxymethyl)pyrazolo[1,5-a]quinoxalin-4(5H)-one. Reaction of 3,6-difluoro-7-(hydroxymethyl)pyrazolo[1,5-a]quinoxalin-4(5H)-one and HBr produced 7-(bromomethyl)-3,6-difluoropyrazolo[1,5-a]quinoxalin-4(5H)-one. Reaction of 7-(bromomethyl)-3,6-difluoropyrazolo[1,5-a]quinoxalin-4(5H)-one and 5-(5,6-dihydropyrrolo[3,4-c]pyrazol-2(4H)-yl)-N-methylpicolinamide under the catalysis of DIEA and KI produced the target compound 5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide.

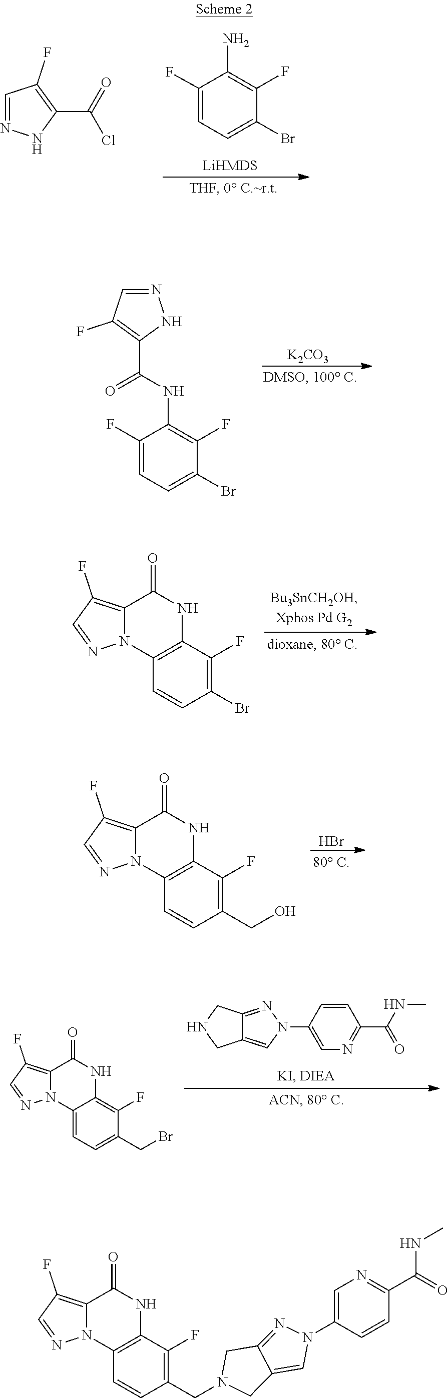

Other related compounds can be prepared using similar methods. For example, replacement of 5-(5,6-dihydropyrrolo[3,4-c]pyrazol-2(4H)-yl)-N-methylpicolinamide with 5-(5,6-dihydropyrrolo[3,4-c]pyrazol-2(4H)-yl)-N,6-dimethylpicolinamide produced the target compound 5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N,6-dimethylpicolinamide. Replacement of 5-(5,6-dihydropyrrolo[3,4-c]pyrazol-2(4H)-yl)-N-methylpicolinamide with N-methyl-5-(4,5,6,7-tetrahydro-2H-pyrazolo[4,3-c]pyridin-2-yl)picolinamide produced the target compound 5-(5-((3,6-difluoro-4-oxo-4,5-dihydropyrazolo[1,5-a]quinoxalin-7-yl)methyl)-4,5,6,7-tetrahydro-2H-pyrazolo[4,3-c]pyridin-2-yl)-N-methylpicolinamide. Replacement of 4-fluoro-1H-pyrazole-5 -carbonyl chloride with 4-(trifluoromethyl)-1H-pyrazole-5-carbonyl chloride produced the target compound 5-(5-((6-fluoro-4-oxo-3-(trifluoromethyl)-4,5-dihydropyrazolo[1,5-a]quinoxalin-7-yl)methyl)-5,6-dihydropyrrolo[3,4-c]pyrazol-2(4H)-yl)-N-methylpicolinamide.

An important aspect of the present disclosure is the discovery that compounds of Formula I (including Formulae II and III) are PARP inhibitors, especially selective PARP1 inhibitors. Therefore, the compounds of Formula I (including Formulae II and III) or stereoisomers, tautomers, N-oxides, hydrates, isotope-substituted derivatives, solvates or pharmaceutically acceptable salts thereof, or mixtures thereof, or prodrugs thereof can be used to treat a variety of diseases or conditions responsive to the inhibition of PARP activity (especially PARP1 activity), or used to prepare a medicament for treating or preventing diseases or conditions responsive to the inhibition of PARP activity (especially PARP1 activity).

In the disclosure, the diseases or conditions responsive to the inhibition of PARP activity (especially PARP1 activity), include cancer. Cancer can be a solid tumor or hematological tumor, including but is not limited to liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, lung cancer (such as small cell lung cancer), Wilms tumor, cervical cancer, testicular cancer, soft tissue sarcoma, primary macroglobulinemia, bladder cancer, chronic myeloid leukemia, primary brain cancer, malignant melanoma, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer. Preferably, the cancer is responsive to the inhibition of PARP activity, especially PARP1 activity.

Therefore, the present disclosure includes methods for the treatment or prevention of diseases or conditions responsive to the inhibition of PARP activity (especially PARP1 activity), comprising administering to a subject (especially mammal, more specifically human) in need thereof an effective amount of the compound of Formula I (including Formulae II and III) or stereoisomers, tautomers, N-oxides, hydrates, isotope-substituted derivatives, solvates or pharmaceutically acceptable salts thereof, or mixtures thereof, or prodrugs thereof, or a pharmaceutical composition comprising an effective amount of the compound of Formula I (including Formulae II and III) or stereoisomers, tautomers, N-oxides, hydrates, isotope-substituted derivatives, solvates or pharmaceutically acceptable salts thereof, or mixtures thereof, or prodrugs thereof. In the disclosure, subjects include mammals, more specifically humans.

In practicing the therapeutic methods, effective amounts of pharmaceutical preparations are administered to an individual exhibiting the symptoms of one or more of these disorders. The pharmaceutic preparations comprise a therapeutically effective amount of the compound of Formula I (including Formulae II and III), formulated for oral, intravenous, local or topical application, for the treatment of cancer and other diseases. The amount is effective to ameliorate or eliminate one or more symptoms of the disorders. An effective amount of a compound for treating a particular disease is an amount that is sufficient to ameliorate or in some manner reduce the symptoms associated with the disease. Such amount may be administered as a single dosage or may be administered according to an effective regimen. The amount may cure the disease but, typically, is administered in order to ameliorate the symptoms of the disease. Typically, repeated administration is required to achieve the desired amelioration of symptom.

In another embodiment, there is provided a pharmaceutical composition comprising a compound of Formula I (including Formulae II and III) as a PARP inhibitor, or stereoisomers, tautomers, N-oxides, hydrates, solvates, isotope-substituted derivatives, or pharmaceutically acceptable salts thereof, or mixtures thereof, or prodrugs thereof.

Another embodiment of the present disclosure is directed to a pharmaceutical composition effective to treat or prevent cancer comprising a compound of Formula I (including Formulae II and III) as a PARP inhibitor, or stereoisomers, tautomers, N-oxides, hydrates, solvates, isotope-substituted derivatives, or pharmaceutically acceptable salts thereof and prodrugs thereof, in combination with at least one known anticancer agent or a pharmaceutically acceptable salt thereof. In particular, the compound herein can be combined with other anticancer drugs related to the mechanism of DNA damage and repair, such as ATM inhibitors, ATR inhibitors, Wee1 inhibitors, DNA-PK inhibitors; HDAC inhibitors such as vorinostat, romidepsin, panobinostat and belinostat; other anticancer drugs related to cell division, including Chk1/2 inhibitors, CDK4/6 inhibitors such as palbociclib; other targeted anticancer agents, including USP1 inhibitors, PRMT5 inhibitors, Polθ inhibitors, RAD51 inhibitors, and so on. Other known anticancer agents which may be used for anticancer combination therapy include, but are not limited to alkylating agents, such as busulfan, melphalan, chlorambucil, cyclophosphamide, ifosfamide, temozolomide, bendamustine, cis-platin, mitomycin C, bleomycin and carboplatin; topoisomerase I inhibitors, such as camptothecin, irinotecan and topotecan; topoisomerase II inhibitors, such as doxorubicin, epirubicin, aclacinomycin, mitoxantrone, elliptinium and etoposide; RNA/DNA antimetabolites, such as 5-azacytidine, gemcitabine, 5-fluorouracil, capecitabine and methotrexate; DNA antimetabolites, such as 5-fluoro-2′-deoxy-uridine, fludarabine, nelarabine, ara-C, pralatrexate, pemetrexed, hydroxyurea and thioguanine; antimitotic agent such as colchicine, vinblastine, vincristine, vinorelbine, paclitaxel, ixabepilone, cabazitaxel and docetaxel; antibodies such as mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin and mabthera; Antibody-Drug Conjugates (ADC) such as T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, and Belantamab Mafodotin; androgen receptor antagonists such as abiraterone, darolutamide, enzalutamide, andprednisone; kinase inhibitors such as imatinib, gefitinib, erlotinib, osimertinib, afatinib, ceritinib, alectinib, crizotinib, erlotinib, lapatinib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, Idelalisib, pazopanib, Torisel and everolimus. Other known anticancer agents which may be used for anticancer combination therapy include tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2) and Sipueucel-T (prostate cancer treatment vaccine).

In practicing the methods of the present disclosure, the compound of the disclosure may be administered together with at least one known anticancer agent in a unitary pharmaceutical composition. Alternatively, the compound of the disclosure may be administered separately from at least one known anticancer agent. In one embodiment, the compound of the disclosure and at least one known anticancer agent are administered substantially simultaneously, i.e. all agents are administered at the same time or one after another, provided that compounds reach therapeutic levels in the blood at the same time. In another embodiment, the compound of the disclosure and at least one known anticancer agent are administered according to individual dose schedule, provided that the compounds reach therapeutic levels in the blood.

Another embodiment of the present disclosure is directed to a bioconjugate to inhibit tumor. The bioconjugate is consisted of the compound described herein and at least one known therapeutically useful antibody, such as trastuzumab or rituximab, or growth factor, such as EGF or FGF, or cytokine, such as IL-2 or IL-4, or any molecule that can bind to cell surface. The antibodies and other molecules could deliver the compound described herein to its targets, making it an effective anticancer agent. The bioconjugates could also enhance the anticancer effect of the therapeutically useful antibodies, such as trastuzumab or rituximab.

Another embodiment of the present disclosure is directed to a pharmaceutical composition effective to inhibit tumor comprising the PARP inhibitor of Formula I (including Formulae II and III), or pharmaceutically acceptable salts thereof, or prodrugs thereof, in combination with radiation therapy. In this embodiment, the compound of the disclosure may be administered at the same time as the radiation therapy or at a different time.

Yet another embodiment of the present disclosure is directed to a pharmaceutical composition effective for post-surgical treatment of cancer, comprising the PARP inhibitor of Formula I (including Formulae II and III), or pharmaceutically acceptable salts thereof, or prodrug thereof. The disclosure also relates to a method of treating cancer by surgically removing tumor and then treating the mammal with the pharmaceutical composition described herein.

Pharmaceutical compositions of this disclosure include all pharmaceutical preparations which contain the compounds of the present disclosure in an amount that is effective to achieve its intended purpose. While individual needs vary, determination of optimal amounts of each component in the pharmaceutical preparations is within the skill of the art. Typically, the compounds or the pharmaceutically acceptable salt thereof may be administered to mammals, orally at a dose of about 0.0025 to 50 mg per kg body weight per day. Preferably, from approximately 0.01 mg/kg to approximately 10 mg/kg body weight is orally administered. If a known anticancer agent is also administered, it is administered in an amount that is effective to achieve its intended purpose. The optimal amounts of such known anticancer agents are well known to those skilled in the art.

The unit oral dose may comprise from approximately 0.01 to approximately 50 mg, preferably approximately 0.1 to approximately 10 mg of the compound of the disclosure. The unit dose may be administered one or more times, with one or more tablets daily, each containing from approximately 0.1 to approximately 50 mg, conveniently approximately 0.25 to 10 mg of the compound of the disclosure or its solvates.

In a topical formulation, the compound of the disclosure may be present at a concentration of approximately 0.01 to 100 mg per gram of carrier.

The compound of the disclosure may be administered as a raw chemical. The compounds of the disclosure may also be administered as part of a suitable pharmaceutical preparation containing pharmaceutically acceptable carriers (comprising excipients and auxiliaries), which facilitate the processing of the compounds into pharmaceutically acceptable preparations. Preferably, the pharmaceutical preparations, particularly oral preparations and those used for the preferred administration, such as tablets, draggers, and capsules, as well as solutions suitable for injection or oral administration, contain from approximately 0.01% to 99%, preferably from approximately 0.25% to 75% of active compound(s), together with excipient(s).

Also included within the scope of the present disclosure are the non-toxic pharmaceutically acceptable salts of the compounds of the present disclosure. Acid addition salts are formed by mixing a solution of the compounds of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, choline hydroxide, sodium carbonate, tris(hydroxymethyl)aminomethane, N-methyl-glucamine and the like.

The pharmaceutical preparations of the disclosure may be administered to any mammal, so long as they may experience the therapeutic effects of the compounds of the disclosure. Foremost among such mammals are humans and veterinary animals, although the disclosure is not intended to be so limited.

The pharmaceutical preparations of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively or concurrently, administration may be by oral route. The dosage administered will be dependent upon the age, health, and weight of the recipient, type of concurrent treatment, frequency of treatment, and the nature of the effect desired.

The pharmaceutical preparations of the present disclosure are manufactured in a known manner, e.g., by means of conventional mixing, granulating, dragee-making, dissolving, or lyophilizing processes. Pharmaceutical preparations for oral use may be obtained by combining the active compounds with solid excipients, optionally grinding the resulting mixture, processing the mixture of granules after adding suitable auxiliaries if desired or necessary, thereby obtaining tablets or dragee cores.

Suitable excipients are, in particular, fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, including, e.g., maize starch, wheat starch, rice starch, potato starch, gelatin, tragacanth, methylcellulose, hydroxypropyl methylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, in particular, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropyl methylcellulose phthalate, are used. Dyes or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate and stabilizers. In soft capsules, the active compounds are preferably dissolved or suspended in suitable liquids, such as fatty oils, or liquid paraffin. In addition, stabilizers may be added.

Suitable formulations for parenteral administration include aqueous solutions of the active compounds, e.g., aqueous solutions and alkaline solutions of water-soluble salts. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, suspension stabilizers may also be contained.

In accordance with one aspect of the present disclosure, compounds of the disclosure are employed in topical and parenteral formulations and are used for the treatment of skin cancer.

The topical formulations of this disclosure are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which the active ingredient, dissolved in a small amount of an oil, such as almond oil, is admixed. A typical example of such a cream is one which includes approximately 40 parts of water, approximately 20 parts of beeswax, approximately 40 parts of mineral oil and approximately 1 part of almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The present disclosure also involves use of the compounds of the disclosure for the manufacture of a medicament for the treatment of clinical symptoms in response to inhibition of the activity of PARP. The medicament may include the above-mentioned pharmaceutical compositions.

The following examples are illustrative, but not limiting, of the method and compositions of the present disclosure. Other suitable modifications and adaptations of the variety of conditions and parameters normally encountered in clinical therapy and which are obvious to those skilled in the art are within the spirit and scope of the disclosure.

### EXAMPLES

### General Remarks

All reagents were of commercial quality. Solvents were dried and purified by standard methods. Mass spectrum analyses were recorded on a Platform II (Agilent 6110) quadrupole mass spectrometer fitted with an electrospray interface. ¹H NMR spectra was recorded at 400 MHz, on a Bricker Ascend 400 apparatus. Chemical shifts were recorded in parts per million (ppm) downfield from TMS (0.00 ppm), and J coupling constants were reported in hertz (Hz).

### Example 1 (1-1 and 1-2)

### 5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide and 5-(5-((6-fluoro-3-methyl-4-oxo-4,5-dihydropyrazolo[1,5-a]quinoxalin-7-yl)methyl)-5,6-dihydropyrrolo[3,4-c]pyrazol-1(4H)-yl)-N-methylpicolinamide

a) Preparation of tert-butyl 2-(6-(methylcarbamoyl)pyridin-3-yl)-2,6-dihydropyrrolo[3,4-c]pyrazole-5(4H)-carboxylate and tert-butyl 1-(6-(methylcarbamoyl)pyridin-3-yl)-4,6 -dihydropyrrolo[3,4-c]pyrazole-5(1H)-carboxylate: To a solution of 5-bromo-N-methylpicolinamide (500 mg, 2.3 mmol) in DMSO (6 mL) was added tert-butyl 2,6-dihydropyrrolo[3,4-c]pyrazole-5(4H)-carboxylate (490 mg, 2.3 mmol), CuI (44 mg, 0.23 mol), K₂CO₃ (965 mg, 6.9 mmol), and tetramethylethylenediamine (TMEDA, 26.7 mg, 0.23 mmol) at room temperature under N₂ atmosphere. The resulting mixture was stirred at 130° C. overnight. The mixture was slowly cooled to room temperature. The resulting reaction mixture was partitioned by adding water (10 mL) and extracted with EA (10 mL×3). The combined organic phase was washed with brine, dried over anhydrous sodium sulfate and concentrated under reduced pressure. The residue was triturated with PE/EA (20:1-10:1-1:1) for three times to give the mixture of products (160 mg, crude, light-yellow solid). MS(ESI): 344.10 [M+H]⁺.
b) Preparation of 5-(5,6-dihydropyrrolo[3,4-c]pyrazol-2(4H)-yl)-N-methylpicolinamide and 5-(5,6-dihydropyrrolo[3,4-c]pyrazol-1(4H)-yl)-N-methylpicolinamide: To a solution of tert-butyl 2-(6-(methylcarbamoyl)pyridin-3-yl)-2,6-dihydropyrrolo[3,4-c]pyrazole-5(4H)-carboxylate and tert-butyl 1-(6-(methylcarbamoyl)pyridin-3-yl)-4,6-dihydropyrrolo[3,4-c]pyrazole-5(1H)-carboxylate (130 mg, 0.38 mmol) in EA (3 mL) was added HCl/EA (1 mL, 3.8 mol). The resulting mixture was stirred at room temperature overnight. After the reaction was completed, the resulting reaction mixture was concentrated under reduced pressure to give the mixture of products (130 mg, crude, light-yellow solid). MS(ESI): 244.05 [M+H]⁺.
c) Preparation of (4-bromo-3-fluoro-2-nitrophenyl)hydrazine: To a solution of 4-bromo-3-fluoro-2-nitroaniline (5.0 g, 21.3 mmol) in concentrated hydrochloric acid (36% aq., 50 mL), a aqueous solution of NaNO₂ (14.4 g, 64.0 mmol) in H₂O (50 mL) was added dropwise at −10° C. The mixture was stirred at −10° C. for 1 hour. The mixture was cooled to −30° C., a solution of SnCl₂·2H₂O (1.6 g, 23.4 mmol) in concentrated hydrochloric acid (36% aq., 50 mL) was added dropwise. The mixture was stirred at −30° C. for 1 hour. After the reaction was completed, the mixture was poured into water (200 mL), the pH of the solution was adjusted to 7-8 with NaHCO₃ solid. The resulting mixture was extracted with EA (100 mL×3). The organic phase was combined and washed with brine, dried over anhydrous sodium sulfate and concentrated under reduced pressure to afford the target product (4.3 g, crude, yellow solid, yield: 81%).
d) Preparation of methyl 1-(4-bromo-3-fluoro-2-nitrophenyl)-4-methyl-1H-pyrazole-5-carboxylate: To a solution of (4-bromo-3-fluoro-2-nitrophenyl)hydrazine (1.0 g, 4.0 mmol) in acetic acid (10 mL), a solution of methyl 4-(dimethylamino)-3-methyl-2-oxobut-3-enoate (0.82 g, 4.8 mmol) in acetic acid (10 mL) was added dropwise at room temperature. The mixture was stirred at 80° C. overnight. After the reaction was completed, the mixture was poured into ice water (100 mL), extracted with EA (100 mL×3). The organic phase was combined and washed with brine, dried over anhydrous sodium sulfate and concentrated under reduced pressure. The residue was purified by silica gel (PE:EA=15:1) to afford the target product (0.83 g, yellow solid, yield: 58%). ¹H NMR (400 MHz, DMSO-d₆): δ 8.16-8.14 (m, 1H), 7.78 (s, 1H), 7.61-7.59 (m, 1H), 3.71 (s, 3H), 2.25 (s, 3H).
e) Preparation of 7-bromo-6-fluoro-3-methylpyrazolo[1,5-a]quinoxalin-4(5H)-one: To a solution of methyl 1-(4-bromo-3-fluoro-2-nitrophenyl)-4-methyl-1H-pyrazole-5-carboxylate (0.83 g, 2.3 mmol) in acetic acid (10 mL), Fe dust (0.65 g, 11.6 mmol, 5.0 eq) was added at room temperature. The mixture was stirred at 80° C. overnight. After the reaction was completed, the mixture was filtered, the filtrate was diluted with water (50 mL) and ethyl acetate (5 mL), then the solid was collected by filtration to afford the target product (370 mg, gray white solid, yield: 55%). MS(ESI): 295.90 [M+H]⁺. H NMR (400 MHz, DMSO-d₆): δ 11.90 (br, 1H), 7.91 (s, 1H), 7.77 (d, J=8.0 Hz, 1H), 7.50-7.46 (m, 1H), 2.38 (s, 3H).
f) Preparation of 6-fluoro-7-(hydroxymethyl)-3-methylpyrazolo[1,5-a]quinoxalin-4(5H)-one: To a solution of 7-bromo-6-fluoro-3-methylpyrazolo[1,5-a]quinoxalin-4(5H)-one (5.0 g, 16.9 mmol) in dioxane (100 mL) was added Bu₃SnCH₂OH (10.9 g, 33.9 mmol) and Xphos Pd G2 (1.3 g, 1.7 mmol) at room temperature. The mixture was stirred at 90° C. under nitrogen atmosphere for 16 hours. After the reaction was completed, an aqueous solution of KF (1 M, 100 mL) was added at room temperature. The mixture was stirred at the same temperature for 10 mins and filtered. The filtrate was extracted with EA (200 mL×3). The combined organic phase was washed with brine (200 mL), dried over anhydrous sodium sulfate and concentrated under reduced pressure. The residue was triturated with a mixture of PE/EA (v/v=1:1) and the solid was collected by filtration to afford the target product (2.8 g, grey solid, yield: 67%). MS(ESI): 248.05 [M+H]⁺.
g) Preparation of 7-(bromomethyl)-6-fluoro-3-methylpyrazolo[1,5-a]quinoxalin-4(5H)-one: To a solution of 6-fluoro-7-(hydroxymethyl)-3-methylpyrazolo[1,5-a]quinoxalin-4(5H)-one (2.8 g, 11.3 mmol) in HBr (30 mL, 48% in H₂O) was stirred at 80° C. for 2 hours. After the reaction was completed, the reaction was concentrated under reduced pressure to afford the target product (2.7 g, crude, yellow solid). MS(ESI): 309.90 [M+H]⁺.
h) Preparation of 5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide and 5-(5-((6-fluoro-3-methyl-4-oxo-4,5-dihydropyrazolo[1,5-a]quinoxalin-7-yl)methyl)-5,6-dihydropyrrolo[3,4-c]pyrazol-1(4H)-yl)-N-methylpicolinamide: To a solution of mixture of 5-(5,6-dihydropyrrolo[3,4-c]pyrazol-2(4H)-yl)-N-methylpicolinamide and 5-(5,6-dihydropyrrolo[3,4-c]pyrazol-1(4H)-yl)-N-methylpicolinamide (95.0 mg, 0.39 mmol) in acetonitrile (3 mL) was added 7-(bromomethyl)-6-fluoro-3-methylpyrazolo[1,5-a]quinoxalin-4(5H)-one (120.0 mg, 0.4 mmol), KI (6.5 mg, 0.039 mol), DIEA (151.0 mg, 1.2 mmol) at room temperature under N₂. The resulting suspension was stirred at 80° C. for 0.5 hour. After the reaction was completed, the solvent was removed under vacuum. The residue was purified by Prep-HPLC (C₁₈, ACN in water, 5-55%, 0.1 HCOOH) to afford the title compounds 5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide (1-1, 3.4 mg, white solid, yield: 1.8%) and 5-(5-((6-fluoro-3-methyl-4-oxo-4,5-dihydropyrazolo[1,5-a]quinoxalin-7-yl)methyl)-5,6-dihydropyrrolo[3,4-c]pyrazol-1(4H)-yl)-N-methylpicolinamide (1-2, 4.7 mg, white solid, yield: 2.5%).

The following compounds of Examples 2-12 were prepared using a synthesis method similar to that described in Example 1 (Scheme 1).

| Ex- | | | | |
|---|---|---|---|---|
| am- | | | MS | |
| ple | Compound | MW | (ESI) | ¹H NMR (400 MHz) |
| 1-1 | 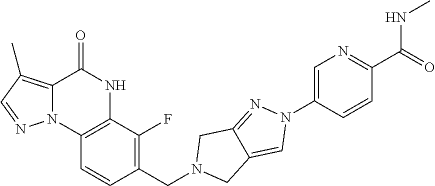 [SMILES: CNC(=O)c1ccc(-n2cc3c(n2)CN(Cc2ccc4c([nH]c(=O)c5c(C)cnn54)c2F)C3)cn1] | 472.48 | 473.20 [M + H]⁺ | DMSO-d₆: δ 11.81 (br, 1H), 9.06 (d, J = 2.5 Hz, 1H), 8.79-8.73 (m, 1H), 8.40 (s, 1H), 8.31 (dd, J = 8.6, 2.6 Hz, 1H), 8.08 (d, J = 8.6 Hz, 1H), 7.93 (s, 1H), 7.88 (d, J = 8.5 Hz, 1H), 7.38 (dd, J = 8.4, 6.9 Hz, 1H), 4.09-4.04 (m, 2H), 3.84 (s, 2H), 3.82-3.78 (m, 2H), 2.80 (d, J = 4.8 Hz, 3H), 2.43 (s, 3H). |
| 1-2 | 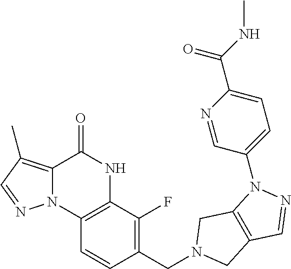 [SMILES: CNC(=O)c1ccc(-n2ncc3c2CN(Cc2ccc4c([nH]c(=O)c5c(C)cnn54)c2F)C3)cn1] | 472.48 | 473.15 [M + H]⁺ | DMSO-d₆: δ 11.78 (br, 1H), 8.85 (s, 1H), 8.76-8.71 (m, 1H), 8.42 (s, 1H), 8.06 (s, 1H), 7.92 (s, 1H), 7.87 (d, J = 8.5 Hz, 1H), 7.61 (s, 1H), 7.38 (t, J = 7.6 Hz, 1H), 4.24-4.22 (m, 2H), 4.08 (s, 2H), 3.85-3.81 (m, 2H), 2.79 (d, J = 4.8 Hz, 3H), 2.42 (s, 3H). |
| 2 | 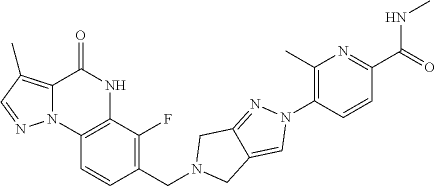 [SMILES: CNC(=O)c1ccc(-n2cc3c(n2)CN(Cc2ccc4c([nH]c(=O)c5c(C)cnn54)c2F)C3)c(C)n1] | 486.51 | 487.30 [M + H]⁺ | DMSO-d₆: δ 11.79 (br, 1H), 8.70-8.64 (m, 1H), 8.16 (s, 1H), 7.95-7.80 (m, 4H), 7.36 (t, J = 7.7 Hz, 1H), 4.04 (s, 2H), 3.81-3.76 (m, 4H), 2.80 (d, J = 4.8 Hz, 3H), 2.53 (s, 3H), 2.40 (s, 3H). |
| 3 | 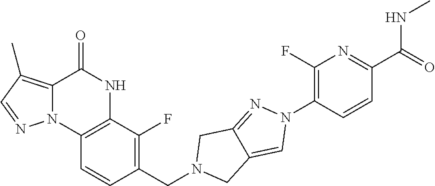 [SMILES: CNC(=O)c1ccc(-n2cc3c(n2)CN(Cc2ccc4c([nH]c(=O)c5c(C)cnn54)c2F)C3)c(F)n1] | 490.47 | 491.15 [M + H]⁺ | DMSO-d₆: δ 11.78 (br, 1H), 8.68 (s, 1H), 8.46-8.36 (m, 1H), 8.06-7.97 (m, 2H), 7.92-7.82 (m, 2H), 7.39-7.30 (m, 1H), 4.04 (s, 2H), 3.81 (s, 2H), 3.76 (s, 2H), 2.77 (d, J = 4.8 Hz, 3H), 2.40 (s, 3H). |
| 4 | 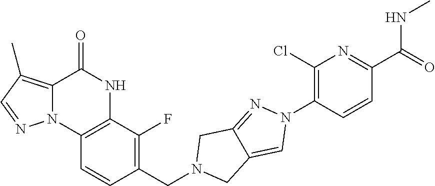 [SMILES: CNC(=O)c1ccc(-n2cc3c(n2)CN(Cc2ccc4c([nH]c(=O)c5c(C)cnn54)c2F)C3)c(Cl)n1] | 506.93 | 507.45 [M + H]⁺ | DMSO-d₆: δ 11.80 (br, 1H), 8.78-8.71 (m, 1H), 8.18 (d, J = 8.1 Hz, 1H), 8.11 (d, J = 8.1 Hz, IH), 8.01 (s, 1H), 7.93 (s, IH), 7.89 (d, J = 8.5 Hz, 1H), 7.40 (t, J = 7.6 Hz, 1H), 4.08 (s, 2H), 3.85-3.79 (m, 4H), 2.83 (d, J = 4.8 Hz, 3H). |
| 5 | 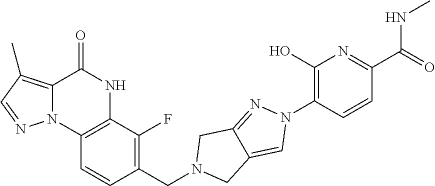 [SMILES: CNC(=O)c1ccc(-n2cc3c(n2)CN(Cc2ccc4c([nH]c(=O)c5c(C)cnn54)c2F)C3)c(O)n1] | 488.48 | 489.20 [M + H]⁺ | DMSO-d₆: δ 8.71-8.62 (m, 1H), 8.55 (s, 1H), 8.27 (s, 1H), 7.97 (d, J = 7.3 Hz, 1H), 7.90 (s, 1H), 7.85 (d, J = 8.6 Hz, 1H), 7.37-7.31 (m, 1H), 6.98 (d, J = 7.7 Hz, 1H), 4.02 (s, 2H), 3.77-3.74 (m, 4H), 2.74 (d, J = 4.4 Hz, 3H), 2.40 (s, 3H). |
| 6 | 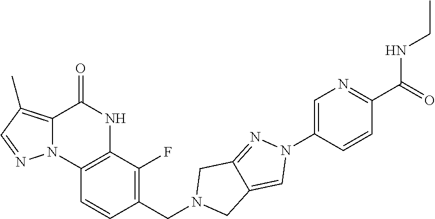 [SMILES: CCNC(=O)c1ccc(-n2cc3c(n2)CN(Cc2ccc4c([nH]c(=O)c5c(C)cnn54)c2F)C3)cn1] | 486.51 | 487.15 [M + H]⁺ | DMSO-d₆: δ 9.03 (d, J = 2.5 Hz, 1H), 8.80 (t, J = 6.1 Hz, 1H), 8.36 (s, 1H), 8.28 (dd, J = 8.6, 2.6 Hz, 1H), 8.06 (d, J = 8.6 Hz, 1H), 7.90 (s, 1H), 7.85 (d, J = 8.6 Hz, 1H), 7.36 (t, J = 7.7 Hz, 1H), 4.04 (s, 2H), 3.82 (s, 2H), 3.79 (s, 2H), 3..31-3.19 (m, 2H), 2.41 (s, 3H), 1.09 (t, J = 7.2 Hz, 3H). |
| 7 | 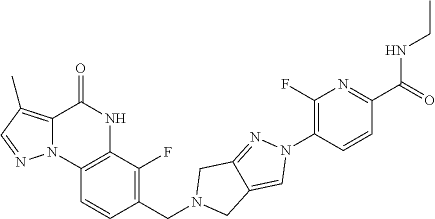 [SMILES: CCNC(=O)c1ccc(-n2cc3c(n2)CN(Cc2ccc4c([nH]c(=O)c5c(C)cnn54)c2F)C3)c(F)n1] | 504.5 | 505.20 [M + H]⁺ | DMSO-d₆: δ 8.74 (t, J = 6.0 Hz, 1H), 8.46 (d, J = 9.5 Hz, 1H), 8.05 (d, J = 12.4 Hz, 2H), 7.93 (s, 1H), 7.89 (d, J = 8.1 Hz, 1H), 7.39 (t, J = 7.5 Hz, 1H), 4.08 (s, 2H), 3.85 (s, 2H), 3.81 (s, 2H), 3.35-3.28 (m, 2H), 2.44 (s, 3H), 1.12 (t, J = 7.0 Hz, 3H). |
| 8 | 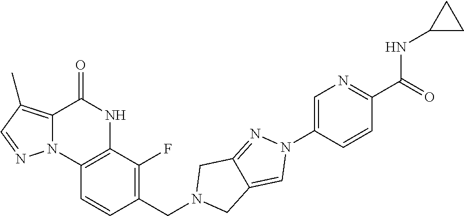 [SMILES: Cc1cnn2c1c(=O)[nH]c1c(F)c(CN3Cc4cn(-c5ccc(C(=O)NC6CC6)nc5)nc4C3)ccc12] | 498.52 | 499.15 [M + H]⁺ | DMSO-d₆: δ 9.02 (d, J = 2.6 Hz, 1H), 8.52 (s, 1H), 8.32 (dd, J = 8.6, 2.6 Hz, 1H), 8.11 (s, 1H), 7.96 (t, J = 4.3 Hz, 2H), 7.51 (t, J = 7.7 Hz, 1H), 4.81 (s, 2H), 4.66 (s, 2H), 4.59 (s, 2H), 2.94-2.77 (m, 1H), 2.41 (s, 3H), 0.72-0.56 (m, 4H). |
| 9 | 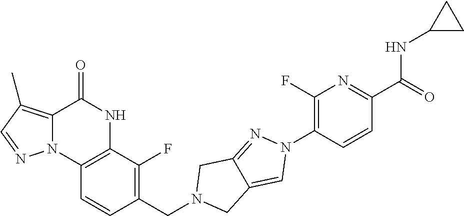 [SMILES: Cc1cnn2c1c(=O)[nH]c1c(F)c(CN3Cc4cn(-c5ccc(C(=O)NC6CC6)nc5F)nc4C3)ccc12] | 516.51 | / | / |
| 10-1 | 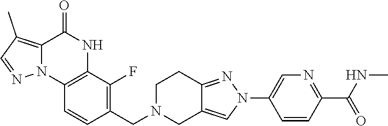 [SMILES: CNC(=O)c1ccc(-n2cc3c(n2)CCN(Cc2ccc4c([nH]c(=O)c5c(C)cnn54)c2F)C3)cn1] | 486.51 | 487.20 [M + H]⁺ | DMSO-d₆: δ 11.79 (br, 1H), 9.03 (d, J = 2.5 Hz, 1H), 8.79-8.70 (m, 1H), 8.39 (s, 1H), 8.28 (dd, J = 8.5, 2.6 Hz, 1H), 8.08 (d, J = 8.6 Hz, 1H), 7.93 (s, 1H), 7.88 (d, J = 8.5 Hz, 1H), 7.35 (t, J = 7.6 Hz, 1H), 3.83 (s, 2H), 3.56 (s, 2H), 2.81 (d, J = 4.8 Hz,3H), 2.80-2.75 (m, 4H), 2.75 (m, 7H), 2.44 (s, 3H). |
| 10-2 | 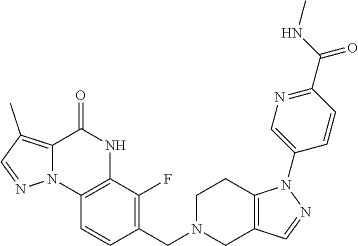 [SMILES: CNC(=O)c1ccc(-n2ncc3c2CCN(Cc2ccc4c([nH]c(=O)c5c(C)cnn54)c2F)C3)cn1] | 486.51 | 487.15 [M + H]⁺ | DMSO-d₆: δ 11.76 (br, 1H), 8.85 (d, J = 2.5 Hz, 1H), 8.82-8.75 (m, 1H), 8.15 (dd, J = 8.4, 2.5 Hz, 1H), 8.08 (d, J = 8.5 Hz, 1H), 7.90 (s, 1H), 7.85 (d, J = 8.4 Hz, IH), 7.59 (s, 1H), 7.37-7.27 (m, 1H), 3.80 (s, 2H), 3.47 (s, 2H), 3.00-2.92 (m, 2H), 2.79 (d, J = 4.8 Hz, 3H), 2.76-2.73 (m, 2H), 2.40 (s, 3H). |
| 11 | 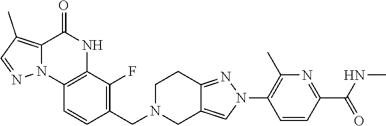 [SMILES: CNC(=O)c1ccc(-n2cc3c(n2)CCN(Cc2ccc4c([nH]c(=O)c5c(C)cnn54)c2F)C3)c(C)n1] | 500.54 | 501.30 [M + H]⁺ | DMSO-d₆: δ 11.79 (s, 1H), 8.71-8.63 (m, 1H), 7.97-7.85 (m, 5H), 7.35 (t, J = 7.6 Hz, 1H), 3.83 (s, 2H), 3.55 (s, 2H), 2.85-2.83 (m, 5H), 2.79-2.73 (m, 2H), 2.57 (s, 3H), 2.44 (s, 3H). |
| 12 | 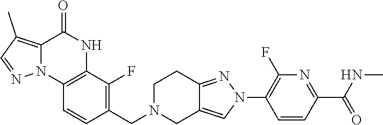 [SMILES: CNC(=O)c1ccc(-n2cc3c(n2)CCN(Cc2ccc4c([nH]c(=O)c5c(C)cnn54)c2F)C3)c(F)n1] | 504.50 | 505.15 [M + H]⁺ | DMSO-d₆: δ 11.73 (br, 1H), δ 8.74-8.65 (m, 1H), 8.46 (t, J = 8.8 Hz, 1H), 8.11-8.02 (m, 2H), 7.93 (s, 1H), 7.88 (d, J = 8.5 Hz, 1H), 7.35 (t, J = 7.7 Hz, 1H), 3.82 (s, 2H), 3.55 (s, 2H), 2.86-2.82 (m, 4H), 2.80 (d, J = 4.7 Hz, 3H), 2.44 (s, 3H). |

### Example 13

### 5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide

a) Preparation of N-(3-bromo-2,6-difluorophenyl)-4-fluoro-1H-pyrazole-5-carboxamide: To a solution of 3-bromo-2,6-difluoroaniline (1.5 g, 7.4 mmol) in THF (15 mL) was added LiHMDS (1M in THF, 20.2 mL, 20.2 mmol) at 0° C. under nitrogen. The mixture was stirred at 0° C. for 30 minutes, 4-fluoro-1H-pyrazole-5-carbonyl chloride (1.0 g, 6.7 mmol) in THF (10 mL) was added dropwise. The reaction mixture was stirred at 0° C. for 30 minutes and warmed to room temperature for 1.5 hours. The mixture was quenched with water (20 mL) and extracted with EA (20 mL×3). The combined organic phase was dried over anhydrous sodium sulfate and concentrated under reduced pressure. The residue was purified by silica gel (PE/EA=5/1) to afford the product (1.5 g, yellow solid, yield: 72%). MS(ESI): 319.95 [M+H]⁺.
b) Preparation of 7-bromo-3,6-difluoropyrazolo[1,5-a]quinoxalin-4(5H)-one: To a solution of N-(3-bromo-2,6-difluorophenyl)-4-fluoro-1H-pyrazole-5-carboxamide (1.5 g, 4.7 mmol) in DMSO (15 mL) was added K₂CO₃ (2.6 g, 18.7 mmol) under nitrogen. The mixture was stirred at 100° C. overnight. The mixture was cooled to room temperature. Water (15 mL) was added, and the mixture was extracted with EA (20 mL×3). Then the organic phase was concentrated under reduced pressure. The residue was purified by flash chromatography (C18, water/acetonitrile=55/45) to give the mixture of the product and its regioisomer (1.2 g, white solid), which was used for the next step directly. MS(ESI): 300.00 [M+H]⁺.
c) Preparation of 3,6-difluoro-7-(hydroxymethyl)pyrazolo[1,5-a]quinoxalin-4(5H)-one: To a solution of 7-bromo-3,6-difluoropyrazolo[1,5-a]quinoxalin-4(5H)-one and its regioisomer (1.2 g, 4.0 mmol) in dried dioxane (12 mL) was added Xphos Pd G2 (314.7 mg, 0.4 mmol) and (tributylstannyl)methanol (3.9 g, 12 mmol) under nitrogen. The mixture was stirred at 80° C. for 4 hours. The mixture was cooled to room temperature and KF aqueous solution (1 M, 24 mL) was added. The resulting mixture was filtered and the filtrate was extracted with EA (20 mL×3). The organic phase was concentrated under reduced pressure. The residue was purified by silica gel (DCM/MeOH=1/0→100/1→80/1→50/1) to give the title compound (260.0 mg, white solid, 2 steps yield: 22%). MS(ESI): 252.10 [M+H]⁺. ¹H NMR (400 MHz, DMSO-d₆): δ 11.94 (s, 1H), 8.20 (d, J=3.8 Hz, 1H), 7.89 (d, J=8.6 Hz, 1H), 7.36 (t, J=7.7 Hz, 1H), 5.44 (t, J=5.8 Hz, 1H), 4.61 (d, J=5.5 Hz, 2H).
d) Preparation of 7-(bromomethyl)-3,6-difluoropyrazolo[1,5-a]quinoxalin-4(5H)-one: A solution of 3,6-difluoro-7-(hydroxymethyl)pyrazolo[1,5-a]quinoxalin-4(5H)-one (200.0 mg, 0.8 mmol) in HBr (48% in water, 20 mL) was stirred at 80° C. for 2 hours under nitrogen. The mixture was concentrated under reduced pressure to give the product (261.0 mg, white solid, yield: 96%). MS(ESI): 313.95 [M+H]⁺.
e) Preparation of 5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide: To a solution of 5-(5,6-dihydropyrrolo[3,4-c]pyrazol-2(4H)-yl)-N-methylpicolinamide (60.0 mg, 0.2 mmol) in ACN (3 mL) was added 7-(bromomethyl)-3,6-difluoropyrazolo[1,5-a]quinoxalin-4(5H)-one (77.0 mg, 0.2 mmol), KI (4.0 mg, 0.02 mol), DIEA (95.0 mg, 0.7 mmol) at room temperature under N₂. The resulting suspension was stirred at 85° C. for 2 hours. After completion of the reaction, the solvent was removed under reduced pressure. The residue was purified by Prep-HPLC to give the title compound (10.3 mg, white solid, yield: 9%).

The following compounds of Examples 14-30, 40-48, 53-62 and 65-66 were prepared using a synthesis method similar to that described in Example 13 (Scheme 2). The following compounds of Examples 31-39, 49-52 and 63-64 were prepared using a synthesis method similar to that described in Example 1 (Scheme 1).

| Ex- | | | | |
|---|---|---|---|---|
| am- | | | MS | |
| ple | Compound | MW | (ESI) | ¹H NMR (400 MHz) |
| 13 | 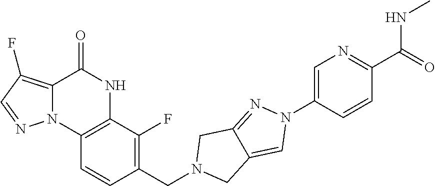 [SMILES: CNC(=O)c1ccc(-n2cc3c(n2)CN(Cc2ccc4c([nH]c(=O)c5c(F)cnn54)c2F)C3)cn1] | 476.45 | 477.10 [M + H]⁺ | DMSO-d₆: δ 9.02 (d, J = 2.6 Hz, 1H), 8.76- 8.67 (m, 1H), 8.36 (s, 1H), 8.27 (dd, J = 8.6, 2.6 Hz, 1H), 8.18 (d, J = 3.7 Hz, 1H), 8.05 (d, J = 8.5 Hz, 1H), 7.88 (d, J = 8.6 Hz, 1H), 7.39 (t, J = 7.7 Hz, 1H), 4.04 (s, 2H), 3.80 (s, 2H), 3.77 (s, 2H), 2.77 (d, J = 4.8 Hz, 3H). |
| 14 | 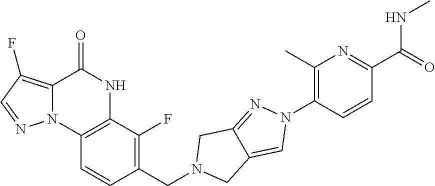 [SMILES: CNC(=O)c1ccc(-n2cc3c(n2)CN(Cc2ccc4c([nH]c(=O)c5c(F)cnn54)c2F)C3)c(C)n1] | 490.47 | 491.15 [M + H]⁺ | DMSO-d₆: δ 8.72-8.63 (m, 1H), 8.29 (s, 1H), 8.19 (d, J = 3.7 Hz, 1H), 7.91-7.86 (m, 4H), 7.44-7.35 (m, 1H), 4.05 (s, 2H), 3.84-3.81 (m, 4H), 2.79 (d, J = 4.8 Hz, 3H), 2.53 (s, 3H). |
| 15 | 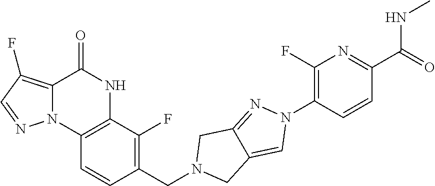 [SMILES: CNC(=O)c1ccc(-n2cc3c(n2)CN(Cc2ccc4c([nH]c(=O)c5c(F)cnn54)c2F)C3)c(F)n1] | 494.44 | 495.15 [M + H]⁺ | DMSO-d₆: δ 11.99 (s, 1H), 8.72-8.64 (m, 1H), 8.41 (t, J = 8.8 Hz, 1H), 8.19 (d, J = 3.7 Hz, 1H), 8.02 (d, J = 7.3 Hz, 2H), 7.89 (d, J = 8.5 Hz, 1H), 7.46-7.37 (m, 1H), 4.07 (s, 2H), 3.91- 3.70 (m, 4H), 2.76 (d, J = 4.7 Hz, 3H). |
| 16 | 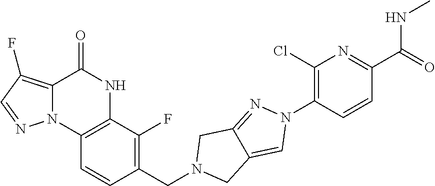 [SMILES: CNC(=O)c1ccc(-n2cc3c(n2)CN(Cc2ccc4c([nH]c(=O)c5c(F)cnn54)c2F)C3)c(Cl)n1] | 510.89 | 511.05 [M + H]⁺ | DMSO-d₆: δ 8.73-8.66 (m, 1H), 8.19-8.16 (m, 1H), 8.16-8.12 (m, 1H), 8.07 (d, J = 8.1 Hz, 1H), 7.97 (s, 1H), 7.88 (d, J = 8.6 Hz, 1H), 7.40 (t, J = 7.6 Hz, 1H), 4.05 (s, 2H), 3.81-3.75 (m, 4H), 2.79 (d, J = 4.8 Hz, 3H). |
| 17 | 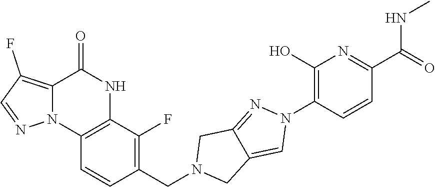 [SMILES: CNC(=O)c1ccc(-n2cc3c(n2)CN(Cc2ccc4c([nH]c(=O)c5c(F)cnn54)c2F)C3)c(O)n1] | 492.45 | 493.10 [M + H]⁺ | DMSO-d₆: δ 8.70-8.64 (m, 1H), 8.54 (s, 1H), 8.23 (s, 1H), 8.19 (d, J = 3.7 Hz, 1H), 7.98 (d, J = 7.6 Hz, 1H), 7.88 (d, J = 8.5 Hz, IH), 7.39 (t, J = 7.6 Hz, 1H), 6.98 (d, J = 7.6 Hz, 1H), 4.04 (s, 2H), 3.78 (s, 2H), 3.74 (s, 2H), 2.74 (d, J = 4.5 Hz, 3H). |
| 18 | 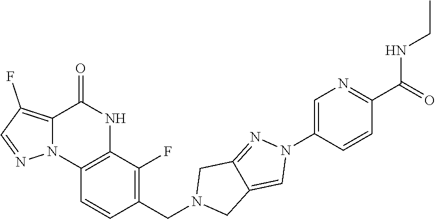 [SMILES: CCNC(=O)c1ccc(-n2cc3c(n2)CN(Cc2ccc4c([nH]c(=O)c5c(F)cnn54)c2F)C3)cn1] | 490.47 | 491.10 [M + H]⁺ | DMSO-d₆: δ 9.03 (d, J = 2.6 Hz, 1H), 8.77 (t, J = 6.2 Hz, 1H), 8.37 (s, 1H), 8.28 (dd, J = 8.5, 2.7 Hz, 1H), 8.19 (d, J = 3.7 Hz, 1H), 8.06 (d, J = 8.6 Hz, 1H), 7.89 (d, J = 8.5 Hz, 1H), 7.40 (dd, J = 8.4, 6.9 Hz, 1H), 4.05 (s, 2H), 3.81 (s, 2H), 3.78 (s, 2H), 3.29-3.23 (m, 2H), 1.08 (t, J = 7.1 Hz, 3H). |
| 19 | 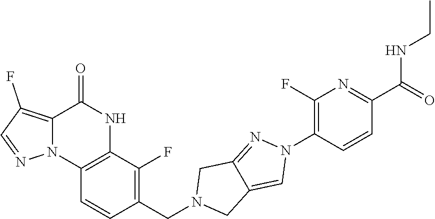 [SMILES: CCNC(=O)c1ccc(-n2cc3c(n2)CN(Cc2ccc4c([nH]c(=O)c5c(F)cnn54)c2F)C3)c(F)n1] | 508.47 | 509.05 [M + H]⁺ | DMSO-d₆: δ 8.77 (t, J = 6.1 Hz, 1H), 8.45 (t, J = 8.8 Hz, 1H), 8.21 (d, J = 3.7 Hz, 1H), 8.10- 8.01 (m, 2H), 7.92 (d, J = 8.5 Hz, 1H), 7.42 (t, J = 7.7 Hz, 1H), 4.09 (s, 2H), 3.85 (s, 2H), 3.81 (s, 2H), 3.32-3.26 (m, 2H), 1.11 (t, J = 7.1 Hz, 3H). |
| 20 | 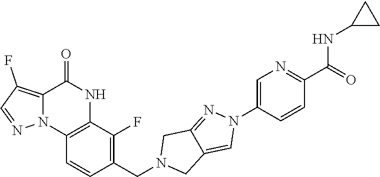 [SMILES: O=C(NC1CC1)c1ccc(-n2cc3c(n2)CN(Cc2ccc4c([nH]c(=O)c5c(F)cnn54)c2F)C3)cn1] | 502.49 | 503.15 [M + H]⁺ | DMSO-d₆: δ 12.05 (br, 1H),8 9.03 (d, J = 2.6 Hz, 1H), 8.74 (d, J = 5.0 Hz, 1H), 8.39 (s, 1H), 8.31 (dd, J = 8.7, 2.6 Hz, 1H), 8.21 (d, J = 3.7 Hz, 1H), 8.09 (d, J = 8.6 Hz, 1H), 7.91 (d, J = 8.5 Hz, 1H), 7.42 (t, J = 7.7 Hz, 1H), 4.08 (s, 2H), 3.85 (s, 2H), 3.81 (s, 2H), 2.96-2.84 (m, 1H), 0.73-0.61 (m, 4H). |
| 21 | 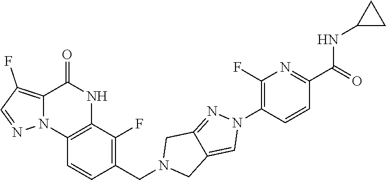 [SMILES: O=C(NC1CC1)c1ccc(-n2cc3c(n2)CN(Cc2ccc4c([nH]c(=O)c5c(F)cnn54)c2F)C3)c(F)n1] | 520.48 | 521.15 [M + H]⁺ | DMSO-d₆: δ 8.63 (s, 1H), 8.41 (t, J = 8.9 Hz, 1H), 8.17 (s, 1H), 8.05-7.77 (m, 2H), 7.88 (d, J = 8.4 Hz, 1H), 7.39 (t, J = 8.4 Hz,, 1H), 4.05 (s, 2H), 3.79 (d, J = 15.2 Hz, 4H), 2.86 (s, 1H), 0.69-0.63 (m, 4H). |
| 22 | 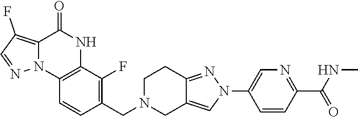 [SMILES: CNC(=O)c1ccc(-n2cc3c(n2)CCN(Cc2ccc4c([nH]c(=O)c5c(F)cnn54)c2F)C3)cn1] | 490.47 | 491.25 [M + H]⁺ | DMSO-d₆: δ 9.04 (d, J = 2.6 Hz, 1H), 8.78- 8.71 (m, 1H), 8.40 (s, 1H), 8.28 (dd, J = 8.6, 2.6 Hz, 1H), 8.22 (d, J = 3.7 Hz, 1H), 8.09 (d, J = 8.6 Hz, 1H), 7.92 (d, J = 8.5 Hz, 1H), 7.43- 7.33 (m, 1H), 3.84 (s, 2H), 3.57 (s, 2H), 2.81 (d, J = 4.7 Hz, 3H), 2.81-2.76 (m, 4H). |
| 23 | 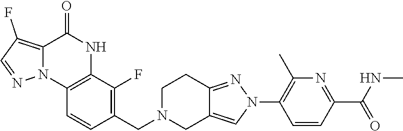 [SMILES: CNC(=O)c1ccc(-n2cc3c(n2)CCN(Cc2ccc4c([nH]c(=O)c5c(F)cnn54)c2F)C3)c(C)n1] | 504.50 | 505.20 [M + H]⁺ | DMSO-d₆: δ 8.71-8.64 (m, 1H), 8.22 (s, 1H), 7.97-7.86 (m, 4H), 7.40 (t, J = 7.8 Hz, 1H), 3.83 (s, 2H), 3.55 (s, 2H), 2.83 (d, J = 4.8 Hz, 3H), 2.83-2.73 (m, 4H), 2.57 (s, 3H). |
| 24 | 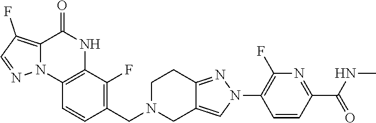 [SMILES: CNC(=O)c1ccc(-n2cc3c(n2)CCN(Cc2ccc4c([nH]c(=O)c5c(F)cnn54)c2F)C3)c(F)n1] | 508.47 | 509.20 [M + H]⁺ | DMSO-d₆: 8.73-8.66 (m, 1H), 8.46 (t, J = 8.8 Hz, 1H), 8.22 (d, J = 3.7 Hz, 1H), 8.12-7.98 (m, 2H), 7.92 (d, J = 8.6 Hz, 1H), 7.39 (t, J = 7.7 Hz, 1H), 3.83 (s, 2H), 3.55 (s, 2H), 2.87- 2.81 (m, 4H), 2.81 (d, J = 4.8 Hz, 3H). |
| 25 | 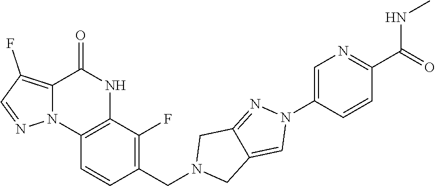 | 526.46 | 527.15 [M + H]⁺ | DMSO-d₆: δ 9.06 (d, J = 2.5 Hz, 1H), 8.80- 8.72 (m, 1H), 8.52 (s, 1H), 8.40 (s, 1H), 8.31 (dd, J = 8.7, 2.7 Hz, 1H), 8.09 (d, J = 8.6 Hz, 1H), 8.01 (d, J = 8.6 Hz, 1H), 7.46 (t, J = 7.6 Hz, 1H), 4.12 (s, 2H), 3.86 (s, 2H), 3.86 (s, 2H), 2.82 (d, J = 4.8 Hz, 3H). |
| 26 | 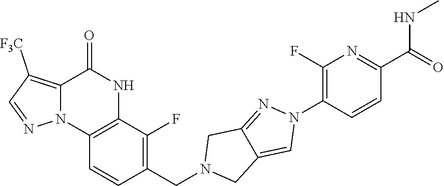 [SMILES: CNC(=O)c1ccc(-n2cc3c(n2)CN(Cc2ccc4c([nH]c(=O)c5c(C(F)(F)F)cnn54)c2F)C3)c(F)n1] | 544.45 | 545.15 [M + H]⁺ | DMSO-d₆: δ 12.42 (s, 1H), 8.75-8.67 (m, 1H), 8.55 (s, 1H), 8.45 (t, J = 8.7 Hz, 1H), 8.10-8.00 (m, 3H), 7.55-7.47 (m, 1H), 4.14 (s, 2H), 3.86 (s, 4H), 2.81 (d, J = 4.7 Hz, 3H). |
| 27 | 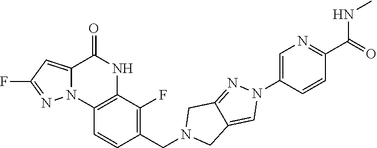 [SMILES: CNC(=O)c1ccc(-n2cc3c(n2)CN(Cc2ccc4c([nH]c(=O)c5cc(F)nn54)c2F)C3)cn1] | 476.45 | 477.25 [M + H]⁺ | DMSO-d₆: δ 9.02 (d, J = 2.3 Hz, 1H), 8.78- 8.66 (m, 1H), 8.35 (s, 1H), 8.28 (d, J = 2.1 Hz, 1H), 8.26 (d, J = 2.5 Hz, 1H), 8.06 (d, J = 8.6 Hz, 1H), 7.74 (d, J = 8.4 Hz, 1H), 7.41-7.34 (m, 1H), 6.90 (d, J = 3.9 Hz, 1H), 4.05 (s, 2H), 3.81 (s, 2H), 3.77 (s, 2H), 2.78 (d, J = 4.8 Hz, 3H). |
| 28 | 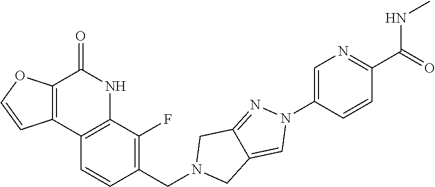 [SMILES: CNC(=O)c1ccc(-n2cc3c(n2)CN(Cc2ccc4c([nH]c(=O)c5occc54)c2F)C3)cn1] | 458.45 | 459.10 [M + H]⁺ | DMSO-d₆: δ 11.91 (s, 1H), 9.06 (d, J = 2.6 Hz, 1H), 8.76 (q, J = 5.2 Hz, IH), 8.39 (s, 1H), 8.31 (dd, J = 8.7, 2.6 Hz, 1H), 8.29 (d, J = 1.9 Hz, 1H), 8.09 (d, J = 8.6 Hz, 1H), 7.85 (d, J = 8.1 Hz, 1H), 7.52 (d, J = 1.9 Hz, 1H), 7.45-7.38 (m, 1H), 4.12 (s, 2H), 3.85 (s, 3H), 3.82 (s, 3H), 2.82 (d, J = 4.8 Hz, 3H). |
| 29 | 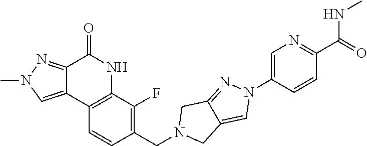 | 472.48 | 473.15 [M + H]⁺ | DMSO-d₆: δ 11.27 (s, 1H), 9.06 (d, J = 2.5 Hz, 1H), 8.75 (d, J = 5.2 Hz, 1H), 8.67 (s, 1H), 8.39 (s, 1H), 8.31 (dd, J = 8.6, 2.6 Hz, 1H), 8.09 (d, J = 8.6 Hz, 1H), 7.69 (d, J = 8.0 Hz, 1H), 7.30 (s, 1H), 4.13 (s, 3H), 4.08 (s, 2H), 3.85 (s, 2H), 3.81 (s, 2H), 2.82 (d, J = 4.8 Hz, 3H). |
| 30 | 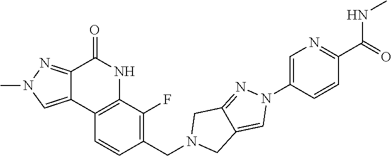 | 472.48 | 473.15 [M + H]⁺ | DMSO-d₆: δ 11.33 (br, 1H), 9.03 (d, J = 2.5 Hz, 1H), 8.74-8.68 (d, J = 5.1 Hz, 1H), 8.31- 8.24 (m, 2H), 8.10 (s, 1H), 8.06 (d, J = 8.6 Hz, 1H), 8.00 (d, J = 8.4 Hz, 1H), 7.39 (t, J = 7.5 Hz, 1H), 4.33 (s, 3H), 4.11 (s, 2H), 3.83 (s, 2H), 3.80 (s, 2H), 2.79 (d, J = 4.8 Hz, 3H). |
| 31 | 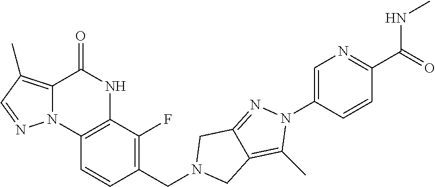 [SMILES: CNC(=O)c1ccc(-n2nc3c(c2C)CN(Cc2ccc4c([nH]c(=O)c5c(C)cnn54)c2F)C3)cn1] | 486.51 | / | / |
| 32 | 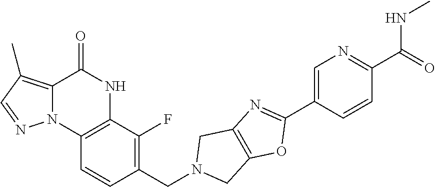 [SMILES: CNC(=O)c1ccc(-c2nc3c(o2)CN(Cc2ccc4c([nH]c(=O)c5c(C)cnn54)c2F)C3)cn1] | 473.47 | 474.15 [M + H]⁺ | DMSO-d₆: δ 9.10 (s, 1H), 8.87 (q, J = 5.0 Hz, 1H), 8.42 (d, J = 8.3 Hz, 1H), 8.11 (d, J = 8.2 Hz, 1H), 7.90 (s, 1H), 7.86 (d, J = 8.7 Hz, 1H), 7.37 (t, J = 7.5 Hz, 1H), 4.08 (s, 2H), 3.98 (s, 2H), 3.81 (s, 2H), 2.79 (d, J = 4.9 Hz, 3H), 2.40 (s, 3H). |
| 33 | 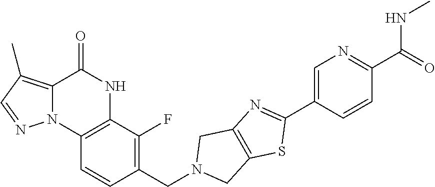 [SMILES: CNC(=O)c1ccc(-c2nc3c(s2)CN(Cc2ccc4c([nH]c(=O)c5c(C)cnn54)c2F)C3)cn1] | 489.53 | 490.10 [M + H]⁺ | DMSO-d₆: δ 9.14 (s, 1H), 8.92-8.84 (m, 1H), 8.49-8.43 (m, 1H), 8.14 (d, J = 8.3 Hz, 1H), 7.97 (s, 1H), 7.93 (d, J = 8.4 Hz, 1H), 7.47-7.41 (m, 1H),4.15 (s, 4H), 4.05 (s, 2H), 2.86 (d, J = 4.4 Hz, 3H), 2.47 (s, 3H). |
| 34 | 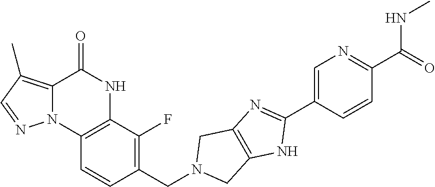 [SMILES: CNC(=O)c1ccc(-c2nc3c([nH]2)CN(Cc2ccc4c([nH]c(=O)c5c(C)cnn54)c2F)C3)cn1] | 472.48 | 473.15 [M + H]⁺ | DMSO-d₆: δ 9.07 (s, 1H), 8.76-8.71 (m, 1H), 8.32-8.28 (s, 2H), 8.02 (d, J = 12.0 Hz, 1H), 7.90 (s, 1H), 7.86 (d, J = 8.0 Hz, 1H), 7.37 (t, J = 8.0 Hz, 1H), 4.06 (s, 2H), 3.83 (brs, 4H), 2.77 (d, J = 4.0 Hz, 3H), 2.41 (s, 3H). |
| 35 | 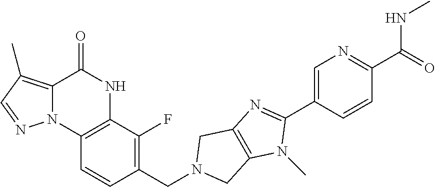 [SMILES: CNC(=O)c1ccc(-c2nc3c(n2C)CN(Cc2ccc4c([nH]c(=O)c5c(C)cnn54)c2F)C3)cn1] | 486.51 | / | / |
| 36 | 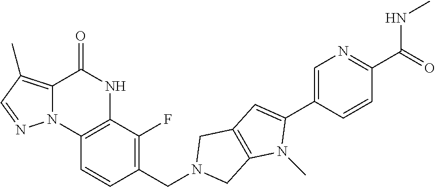 [SMILES: CNC(=O)c1ccc(-c2cc3c(n2C)CN(Cc2ccc4c([nH]c(=O)c5c(C)cnn54)c2F)C3)cn1] | 485.52 | / | / |
| 37 | 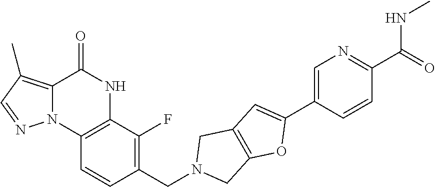 [SMILES: CNC(=O)c1ccc(-c2cc3c(o2)CN(Cc2ccc4c([nH]c(=O)c5c(C)cnn54)c2F)C3)cn1] | 472.48 | / | / |
| 38 | 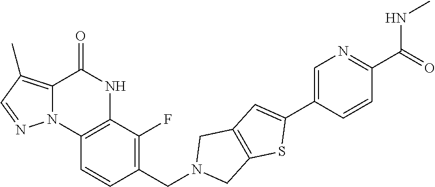 [SMILES: CNC(=O)c1ccc(-c2cc3c(s2)CN(Cc2ccc4c([nH]c(=O)c5c(C)cnn54)c2F)C3)cn1] | 488.54 | / | / |
| 39 | 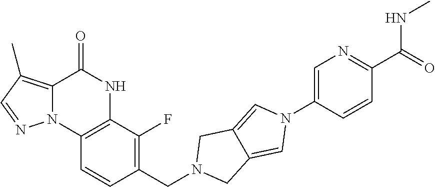 [SMILES: CNC(=O)c1ccc(-n2cc3c(c2)CN(Cc2ccc4c([nH]c(=O)c5c(C)cnn54)c2F)C3)cn1] | 471.50 | 472.25 [M + H]⁺ | DMSO-d₆: δ 8.85 (d, J = 2.6 Hz, 1H), 8.67 (d, J = 5.2 Hz, 1H), 8.11 (dd, J = 9.0, 2.4 Hz, 1H), 8.03 (d, J = 8.4 Hz, 1H), 7.93 (s, 1H), 7.88 (d, J = 8.6 Hz, 1H), 7.38 (t, J = 7.6 Hz, 1H), 7.29 (s, 2H), 4.01 (s, 2H), 3.72 (s, 4H), 2.82 (d, J = 4.8 Hz, 3H), 2.44 (s, 3H). |
| 40 | 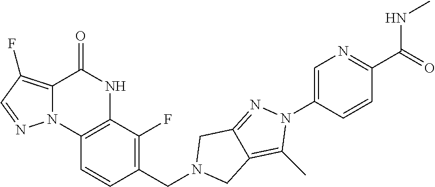 [SMILES: CNC(=O)c1ccc(-n2nc3c(c2C)CN(Cc2ccc4c([nH]c(=O)c5c(F)cnn54)c2F)C3)cn1] | 490.47 | 491.15 [M + H]⁺ | DMSO-d₆: δ 8.80-8.74 (m, 2H), 8.19 (d, J = 3.7 Hz, 1H), 8.11 (d, J = 1.6 Hz, 2H), 7.91 (d, J = 8.5 Hz, 1H), 7.41 (dd, J = 8.5, 6.8 Hz, 1H), 4.06 (s, 2H), 3.80 (s, 2H), 3.72 (s, 2H), 2.82 (d, J = 4.8 Hz, 3H), 2.33 (s, 3H). |
| 41 | 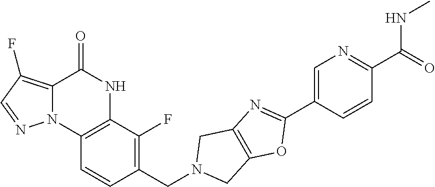 [SMILES: CNC(=O)c1ccc(-c2nc3c(o2)CN(Cc2ccc4c([nH]c(=O)c5c(F)cnn54)c2F)C3)cn1] | 477.43 | 478.15 [M + H]⁺ | DMSO-d₆: δ 9.10 (s, 1H), 8.87 (q, J = 5.0 Hz, 1H), 8.42 (d, J = 8.3 Hz, 1H), 8.17 (d, J = 3.7 Hz, 1H), 8.11 (d, J = 8.4 Hz, 1H), 7.89 (d, J = 8.5 Hz, 1H), 7.40 (t, J = 7.5 Hz, 2H), 4.09 (s, 2H), 3.98 (d, J = 4.0 Hz, 2H), 3.81 (s, 2H), 2.79 (d, J = 4.8 Hz, 3H). |
| 42 | 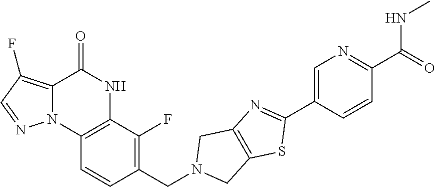 [SMILES: CNC(=O)c1ccc(-c2nc3c(s2)CN(Cc2ccc4c([nH]c(=O)c5c(F)cnn54)c2F)C3)cn1] | 493.49 | 494.25 [M + H]⁺ | DMSO-d₆: δ 9.10 (d, J = 2.2 Hz, 1H), 8.83 (q, J = 4.7 Hz, 1H), 8.41 (dd, J = 8.2, 2.3 Hz, 1H), 8.21 (d, J = 3.7 Hz, 1H), 8.11 (d, J = 8.3 Hz, 1H), 7.97-7.90 (m, 1H), 7.44 (dd, J = 8.6, 6.8 Hz, 1H), 4.17-4.09 (m, 4H), 4.01 (t, J = 3.2 Hz, 2H), 2.83 (d, J = 4.8 Hz, 3H). |
| 43 | 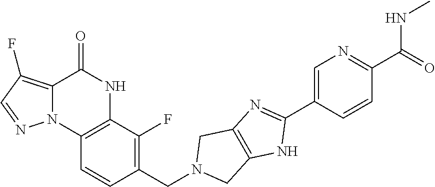 [SMILES: CNC(=O)c1ccc(-c2nc3c([nH]2)CN(Cc2ccc4c([nH]c(=O)c5c(F)cnn54)c2F)C3)cn1] | 476.45 | 477.10 [M + H]⁺ | DMSO-d₆: δ 9.07 (s, 1H), 8.76-8.71 (m, 1H), 8.32-8.28 (s, 1H), 8.22-8.20 (m, 2H), 8.02 (d, J = 8.0 Hz, 1H), 7.89 (d, J = 8.0 Hz, 1H), 7.42 (t, J = 8.0 Hz, 1H), 4.06 (s, 2H), 3.83 (brs, 4H), 2.77 (d, J = 4.0 Hz, 3H). |
| 44 | 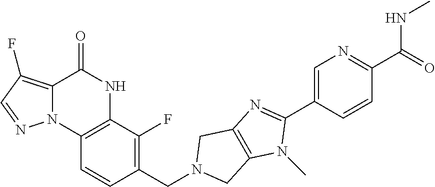 [SMILES: CNC(=O)c1ccc(-c2nc3c(n2C)CN(Cc2ccc4c([nH]c(=O)c5c(F)cnn54)c2F)C3)cn1] | 490.47 | 491.15 [M + H]⁺ | DMSO-d₆: δ 8.88 (d, J = 2.1 Hz, 1H), 8.78 (q, J = 4.9 Hz, 1H), 8.24-8.14 (m, 2H), 8.04 (d, J = 8.2 Hz, 1H), 7.89 (d, J = 8.5 Hz, 1H), 7.41 (t, J = 7.6 Hz, 1H), 4.06 (s, 2H), 3.88 (s, 2H), 3.78 (s, 2H), 3.69 (s, 3H), 2.80 (d, J = 4.8 Hz, 3H). |
| 45 | 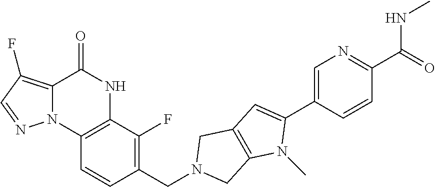 [SMILES: CNC(=O)c1ccc(-c2cc3c(n2C)CN(Cc2ccc4c([nH]c(=O)c5c(F)cnn54)c2F)C3)cn1] | 489.49 | / | / |
| 46 | 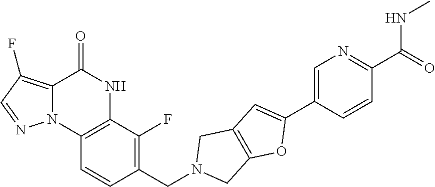 [SMILES: CNC(=O)c1ccc(-c2cc3c(o2)CN(Cc2ccc4c([nH]c(=O)c5c(F)cnn54)c2F)C3)cn1] | 476.44 | 477.15 [M + H]⁺ | DMSO-d₆: δ 11.95 (s, 1H),8.91 (s, 1H), 8.71 (q, J = 5.3 Hz, 1H), 8.20 (d, J = 3.7 Hz, 1H), 8.19-8.15 (m, 1H), 8.01 (d, J = 8.2 Hz, 1H), 7.92 (d, J = 8.5 Hz, 1H), 7.42 (s, 1H), 7.24 (s, 1H), 4.09 (s, 2H), 3.94 (s, 2H), 3.88 (s, 2H), 2.82 (d, J = 4.8 Hz, 3H). |
| 47 | 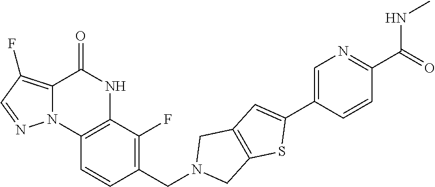 [SMILES: CNC(=O)c1ccc(-c2cc3c(s2)CN(Cc2ccc4c([nH]c(=O)c5c(F)cnn54)c2F)C3)cn1] | 492.50 | 493.10 [M + H]⁺ | DMSO-d₆: δ 8.86 (s, 1H), 8.71 (q, J = 5.3 Hz, 1H), 8.38 (s, 2H), 8.20 (d, J = 3.7 Hz, 1H), 8.16-8.10 (m, 1H), 8.01 (d, J = 8.2 Hz, 1H), 7.92 (d, J = 8.5 Hz, 1H), 7.56 (s, 1H), 7.43 (t, J = 7.7 Hz, 1H), 4.08 (s, 2H), 4.03 (s, 2H), 3.88 (s, 2H), 2.82 (d, J = 4.8 Hz, 3H). |
| 48 | 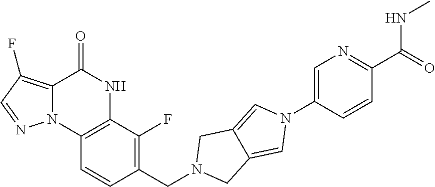 [SMILES: CNC(=O)c1ccc(-n2cc3c(c2)CN(Cc2ccc4c([nH]c(=O)c5c(F)cnn54)c2F)C3)cn1] | 475.46 | 476.15 [M + H]⁺ | DMSO-d₆: δ 8.85 (d, J = 2.6 Hz, 1H), 8.67 (q, J = 5.1 Hz, 1H), 8.21 (d, J = 3.7 Hz, 1H), 8.12- 8.07 (m, 1H), 8.03 (d, J = 8.6 Hz, 1H), 7.92 (d, J = 8.5 Hz, 1H), 7.42 (t, J = 7.6 Hz, 1H), 7.29 (s, 2H), 4.02 (s, 2H), 3.72 (s, 4H), 2.82 (d, J = 4.8 Hz, 3H). |
| 49 | 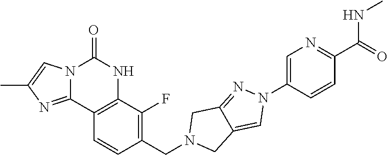 | 472.48 | 473.10 [M + H]⁺ | DMSO-d₆: δ 9.06 (d, J = 2.5 Hz, 1H), 8.75 (q, J = 5.1 Hz,1H), 8.39 (s, 1H), 8.31 (dd, J = 8.6, 2.6 Hz, 1H), 8.09 (d, J = 8.6 Hz, 1H), 7.89 (d, J = 8.1 Hz, 1H), 7.64 (d, J = 1.4 Hz, 1H), 7.42 (dd, J = 8.1, 6.4 Hz, 1H), 4.11 (s, 2H), 3.86 (s, 2H), 3.82 (s, 2H), 2.82 (d, J = 4.7 Hz,3H), 2.31 (s, 3H). |
| 50 | 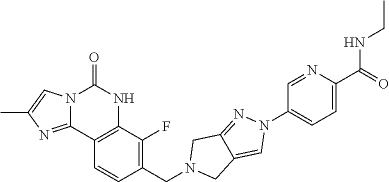 [SMILES: CCNC(=O)c1ccc(-n2cc3c(n2)CN(Cc2ccc4c([nH]c(=O)n5cc(C)nc45)c2F)C3)cn1] | 486.51 | 487.10 [M + H]⁺ | DMSO-d₆: δ 9.06 (d, J = 2.5 Hz, 1H), 8.80 (t, J = 6.5 Hz, 1H), 8.39 (s, 1H), 8.35 - 8.23 (m, 1H), 8.10 (d, J = 9.0 Hz, 1H), 7.89 (d, J = 8.6 Hz, 1H), 7.64 (s, 1H), 7.47-7.36 (m, 1H), 4.11 (s, 2H), 3.84 (d, J = 15.2 Hz, 4H), 3.32- 3.27 (m, 2H), 2.31 (s, 3H), 1.12 (t, J = 7.1 Hz, 3H). |
| 51 | 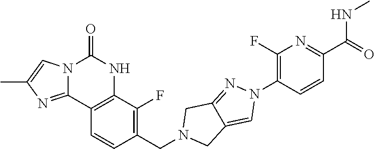 | 490.47 | 491.20 [M + H]⁺ | DMSO-d₆: δ 8.67 (q, J = 5.2 Hz, 1H), 8.41 (t, J = 8.8 Hz, 1H), 8.05-7.97 (m, 2H), 7.85 (d, J = 8.2 Hz, 1H), 7.60 (s, 1H), 7.38 (t, J = 7.3 Hz, 1H), 4.07 (s, 2H), 3.81 (s, 2H), 3.77 (s, 2H), 2.77 (d, J = 4.8 Hz, 3H), 2.27 (s, 3H). |
| 52 | 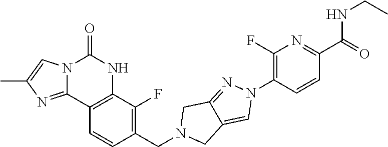 [SMILES: CCNC(=O)c1ccc(-n2cc3c(n2)CN(Cc2ccc4c([nH]c(=O)n5cc(C)nc45)c2F)C3)c(F)n1] | 504.50 | 505.15 [M + H]⁺ | DMSO-d₆: δ 8.76 (t, J = 5.5 Hz, 1H), 8.46 (t, 1H), 8.10-8.01 (m, 2H), 7.89 (d, J = 8.1 Hz, 1H), 7.63 (s, 1H), 7.41 (t, J = 7.2 Hz, 1H), 4.11 (s, 2H), 3.86 (s, 2H), 3.81 (s, 2H), 3.32-3.27 (m, 2H), 2.31 (s, 3H), 1.11 (t, J = 7.2 Hz, 3H). |
| 53 | 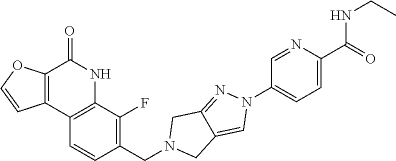 [SMILES: CCNC(=O)c1ccc(-n2cc3c(n2)CN(Cc2ccc4c([nH]c(=O)c5occc54)c2F)C3)cn1] | 472.48 | 473.15 [M + H]⁺ | DMSO-d₆: δ 11.91 (s, 1H), 9.07 (d, J = 2.5 Hz, 1H), 8.79 (t, J = 6.1 Hz, 1H), 8.40 (s, 1H), 8.35-8.30 (m, 1H), 8.29 (s, 1H), 8.10 (d, J = 8.6 Hz, 1H), 7.86 (d, J = 8.2 Hz, 1H), 7.52 (s, 1H), 7.42 (t, J = 7.6 Hz, 1H), 4.14 (s, 2H), 4.01-3.69 (m, 4H), 3.33-3.28 (m, 2H), 1.12 (t, J = 7.2 Hz, 3H). |
| 54 | 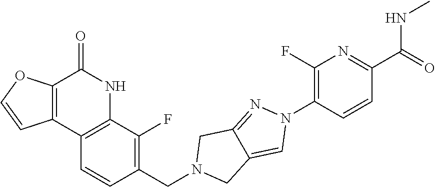 [SMILES: CNC(=O)c1ccc(-n2cc3c(n2)CN(Cc2ccc4c([nH]c(=O)c5occc54)c2F)C3)c(F)n1] | 476.44 | 477.00 [M + H]⁺ | DMSO-d₆: δ 11.86 (s, 1H), 8.66 (q, J = 4.9 Hz, 1H), 8.42 (t, J = 8.0 Hz, 1H), 8.24 (d, J = 1.7 Hz, 1H), 8.03-8.00 (m, 2H), 7.82 (d, J = 8.0 Hz, 1H), 7.47 (d, J = 1.7 Hz, 1H), 7.37 (t, J = 8.0 Hz, 1H), 4.08 (s, 2H), 3.82 (s, 2H), 3.77 (s, 2H), 2.76 (d, J = 4.0 Hz, 3H). |
| 55 | 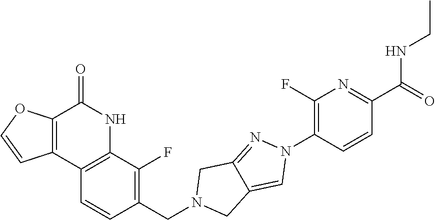 [SMILES: CCNC(=O)c1ccc(-n2cc3c(n2)CN(Cc2ccc4c([nH]c(=O)c5occc54)c2F)C3)c(F)n1] | 490.47 | 491.15 [M + H]⁺ | DMSO-d₆: δ 11.86 (s, 1H), 8.72 (t, J = 4.0 Hz, 1H), 8.42 (t, J = 8.0 Hz, 1H), 8.24 (s, 1H), 8.03-8.00 (m, 2H), 7.82 (d, J = 8.0 Hz, 1H), 7.48 (s, 1H), 7.38 (t, J = 8.0 Hz, 1H), 4.09 (s, 2H), 3.82 (s, 2H), 3.78 (s, 2H), 3.31-3.28 (m, 2H), 1.08 (t, J = 8.0 Hz, 3H). |
| 56 | 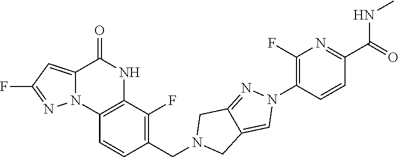 [SMILES: CNC(=O)c1ccc(-n2cc3c(n2)CN(Cc2ccc4c([nH]c(=O)c5cc(F)nn54)c2F)C3)c(F)n1] | 494.44 | 495.25 [M + H]⁺ | DMSO-d₆: δ 8.65 (q, J = 4.8 Hz, 1H), 8.42 (t, J = 8.7 Hz, 1H), 8.04-7.98 (m, 2H), 7.76 (d, J = 8.5 Hz, 1H), 7.41 (t, J = 7.7 Hz, 1H), 6.92 (d, J = 5.5 Hz, 1H), 4.07 (s, 2H), 3.82 (s, 2H), 3.78 (s, 2H), 2.78 (d, J = 4.8 Hz, 3H). |
| 57 | 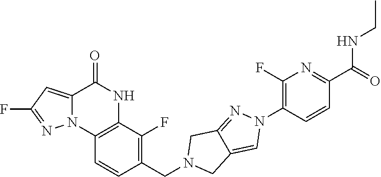 [SMILES: CCNC(=O)c1ccc(-n2cc3c(n2)CN(Cc2ccc4c([nH]c(=O)c5cc(F)nn54)c2F)C3)c(F)n1] | 508.47 | 509.05 [M + H]⁺ | DMSO-d₆: δ 8.72 (t, J = 6.4 Hz,1H), 8.41 (t, J = 8.7 Hz, 1H), 8.07-7.94 (m, 2H), 7.74 (d, J = 8.5 Hz, 1H), 7.39 (t, J = 7.7 Hz, 1H), 6.91 (d, J = 5.5 Hz, 1H), 4.05 (s, 2H), 3.81 (s, 2H), 3.77 (s, 2H), 3.32-3.27-3 (m, 2H), 1.08 (t, J = 7.1 Hz, 3H). |
| 58 | 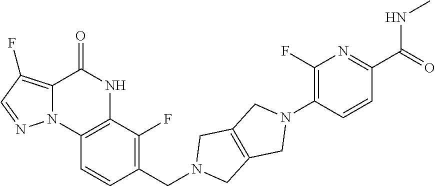 [SMILES: CNC(=O)c1ccc(N2CC3=C(CN(Cc4ccc5c([nH]c(=O)c6c(F)cnn65)c4F)C3)C2)c(F)n1] | 495.47 | 496.00 [M + H]⁺ | DMSO-d₆: δ 8.20-8.16 (m, 2H), 7.88 (d, J = 8.6 Hz, 1H), 7.73 (dd, J = 8.1, 1.9 Hz, 1H), 7.37 (dd, J = 8.5, 6.8 Hz, 1H), 7.08 (dd, J = 10.8, 8.3 Hz, 1H), 4.25 (d, J = 2.8 Hz, 4H), 3.98 (s, 2H), 3.54 (s, 4H), 2.72 (d, J = 4.8 Hz, 3H). |
| 59 | 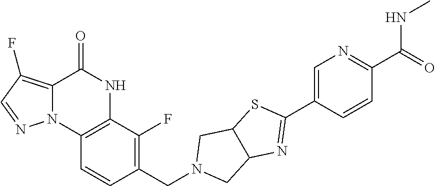 [SMILES: CNC(=O)c1ccc(C2=NC3CN(Cc4ccc5c([nH]c(=O)c6c(F)cnn65)c4F)CC3S2)cn1] | 495.51 | 496.15 [M + H]⁺ | DMSO-d₆: δ 9.00 (d, J = 2.1 Hz, 1H), 8.76 (q, J = 4.5 Hz, 1H), 8.33-8.24 (m, 1H), 8.20 (d, J = 3.7 Hz, 1H), 8.08 (d, J = 8.2 Hz, 1H), 7.86 (d, J = 8.4 Hz, 1H), 7.27 (t, J = 7.7 Hz, 1H), 5.48-5.35 (m, 1H), 4.33-4.28 (m, 1H), 3.80- 3.69 (m, 2H), 2.95-2.85 (m, 2H), 2.81 (d, J = 4.5 Hz, 3H), 2.73-2.68 (m, 1H), 2.55-2.52 (m, 1H). |
| 60 | 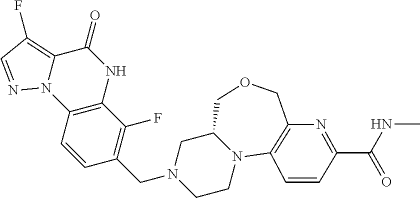 [SMILES: CNC(=O)c1ccc2c(n1)COC[C@H]1CN(Cc3ccc4c([nH]c(=O)c5c(F)cnn54)c3F)CCN21] | 495.49 | 496.60 [M + H]⁺ | DMSO-d₆: δ 8.40 (q, J = 5.2 Hz, 1H), 8.21 (d, J = 3.7 Hz, 1H), 7.91 (d, J = 8.5 Hz, 1H), 7.81 (d, J = 8.4 Hz, 1H), 7.48 (d, J = 8.5 Hz, 1H), 7.34 (t, J = 7.7 Hz, 1H), 4.92 (d, J = 13.8 Hz, 1H), 4.74 (d, J = 13.9 Hz, 1H), 3.98-3.92 (m, 1H), 3.80-3.74 (m, 1H), 3.67 (s, 2H), 3.49-3.42 (m, 2H), 2.78 (d, J = 4.8 Hz, 3H), 2.71-2.58 (m, 2H), 2.55-2.52 (m, 1H), 2.45-2.38 (m, 2H). |
| 61 | 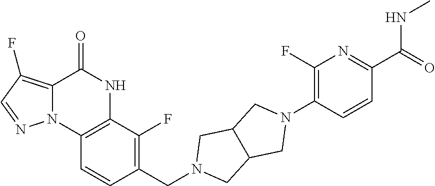 [SMILES: CNC(=O)c1ccc(N2CC3CN(Cc4ccc5c([nH]c(=O)c6c(F)cnn65)c4F)CC3C2)c(F)n1] | 497.48 | 498.20 [M + H]⁺ | DMSO-d₆: δ 8.28-8.20 (m, 1H), 8.17 (d, J = 3.7 Hz, 1H), 7.84 (d, J = 8.5 Hz, 1H), 7.84 (d, J = 8.6 Hz, 1H),7.32-7.18 (m, 2H), 3.67 (s, 2H), 3.52-3.42 (m, 2H), 3.25-3.18 (m, 2H), 2.888-2.81 (m, 2H), 2.72 (d, J = 4.7 Hz, 3H), 2.69-2.62 (m, 2H), 2.45-2.39 (m, 2H). |
| 62 | 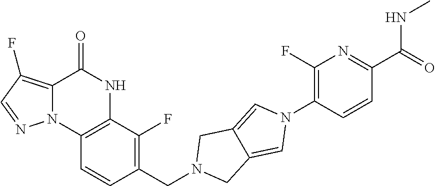 [SMILES: CNC(=O)c1ccc(-n2cc3c(c2)CN(Cc2ccc4c([nH]c(=O)c5c(F)cnn54)c2F)C3)c(F)n1] | 493.45 | 494.15 [M + H]⁺ | DMSO-d₆: δ 8.63 (q, J = 4.8 Hz, 1H), 8.20- 8.15 (m, 2H), 7.96 (d, J = 8.0 Hz, 1H), 7.87 (d, J = 8.5 Hz, 1H), 7.38 (t, J = 7.7 Hz, 1H), 7.00 (s, 2H), 3.98 (s, 2H), 3.68 (s, 3H), 2.77 (d, J = 4.7 Hz, 4H). |
| 63 | 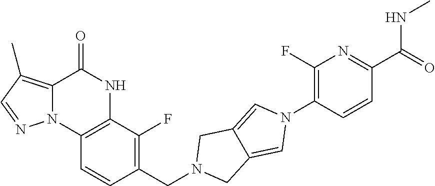 [SMILES: CNC(=O)c1ccc(-n2cc3c(c2)CN(Cc2ccc4c([nH]c(=O)c5c(C)cnn54)c2F)C3)c(F)n1] | 489.49 | 490.20 [M + H]⁺ | DMSO-d₆: δ 8.64 (q, J = 4.8 Hz, 1H), 8.24- 8.14 (m, 1H), 7.96 (d, J = 8.0 Hz, 1H), 7.90 (s, 0H), 7.85 (d, J = 8.3 Hz, 1H), 7.39-7.31 (m, 1H), 7.00 (s, 2H), 3.98 (s, 2H), 3.68 (s, 4H), 2.77 (d, J = 4.7 Hz, 3H), 2.41 (s, 3H). |
| 64 | 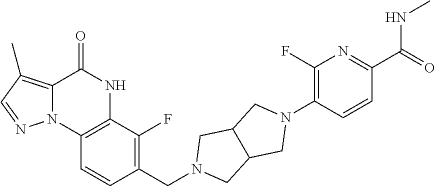 [SMILES: CNC(=O)c1ccc(N2CC3CN(Cc4ccc5c([nH]c(=O)c6c(C)cnn65)c4F)CC3C2)c(F)n1] | 493.52 | 494.10 [M + H]⁺ | DMSO-d₆: δ 8.28 (q, J = 5.1 Hz, 1H), 7.84 (d, J = 8.5 Hz, 1H), 7.77 (dd, J = 8.1, 1.8 Hz, 1H), 7.31-7.23 (m, 2H), 3.69 (s, 2H), 3.53 (t, J = 8.3 Hz, 2H), 3.23 (d, J = 10.1 Hz, 2H), 2.91- 2.83 (m, 2H), 2.75 (d, J = 4.8 Hz, 3H), 2.70 (t, J = 7.6 Hz, 2H), 2.48 - 2.43 (m, 2H), 2.43 (s, 3H). |
| 65 | 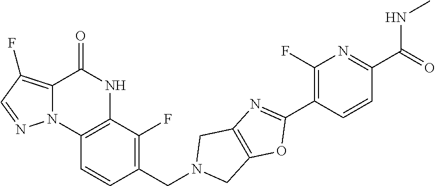 [SMILES: CNC(=O)c1ccc(-c2nc3c(o2)CN(Cc2ccc4c([nH]c(=O)c5c(F)cnn54)c2F)C3)c(F)n1] | 495.42 | / | / |
| 66 | 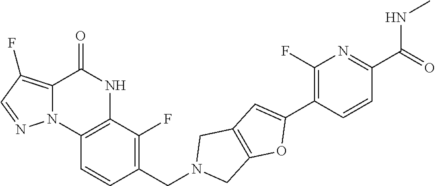 [SMILES: CNC(=O)c1ccc(-c2cc3c(o2)CN(Cc2ccc4c([nH]c(=O)c5c(F)cnn54)c2F)C3)c(F)n1] | 494.43 | / | / |

### Example 67

### PARP1 and PARP2 Chemiluminescent Assay

The solution of recombinant poly(ADP-ribose) polymerase 1 and 2 (PARP1 and PARP2) (40 ng enzyme/well) and the compounds to be tested were mixed, respectively. The solutions were added to a 96-well plate coated with histone mixture, incubated at room temperature for 1 h, then 50 μL 0.3 ng/mL Streptavidin-HRP was added to each well. The plates were incubated for 30 minutes at room temperature. Finally, the plates were treated with streptavidin-HRP followed by addition of the ELISA ECL substrate to produce chemiluminescence that can be measured using a chemiluminescence reader. Inhibition of the tested compound to PARP1/2 enzyme activity was calculated according to the following formula.

$$Inhibition ( \% ) = \frac{Readings of positive control - X}{Readings of positive control - Readings of negitive control}$$

IC₅₀ value is obtained by fitting the s-shaped dose response curve equation by using XL Fit software. The curve equation is Y=100/(1+10\hat{ }(log C-log IC₅₀)), C is the compound concentration.

Table 1 summarize the inhibitory effects of compounds on PARP1 and PARP2 enzyme activity (IC₅₀).

TABLE 1

IC₅₀ (nM)

| Example No. | PARP1 | PARP2 |
|---|---|---|
| 1-1 | 0.47 | >10000 |
| 1-2 | 204 | >1000 |
| 6 | 0.38 | >10000 |
| 8 | 0.94 | >10000 |
| 10-1 | 245 | >10000 |
| 10-2 | 120 | >10000 |
| 13 | 0.16 | 42 |
| 14 | 0.23 | 1158 |
| 15 | 0.24 | 373 |
| 16 | 0.32 | 1191 |
| 18 | 0.23 | 170 |
| 19 | 0.22 | >10000 |
| 20 | 0.11 | 68 |
| 21 | 0.32 | >10000 |
| 22 | 81 | >10000 |
| 25 | 0.26 | 21 |
| 26 | 0.46 | 64 |
| 27 | 0.34 | 217 |
| 28 | 0.34 | 119 |
| 29 | 0.29 | >10000 |
| 30 | 0.25 | 48 |
| 34 | 0.38 | 40 |
| 39 | 0.18 | 167 |
| 40 | 85 | / |
| 41 | 0.21 | 61.15 |
| 42 | 0.53 | 118 |
| 43 | 0.34 | 5.18 |
| 46 | 0.19 | 32.80 |
| 48 | 0.11 | 48 |
| 49 | 0.36 | 107 |
| 50 | 0.39 | 180 |
| 51 | 0.75 | >10000 |
| 52 | 0.42 | 1079 |
| 53 | 0.17 | 157 |
| 54 | 0.12 | 255 |
| 55 | 0.20 | 403 |
| 56 | 0.22 | 2764.54 |
| 57 | 0.74 | >10000 |
| 58 | 0.38 | 1619 |
| 59 | 0.79 | 84 |
| 62 | 0.12 | 278.15 |
| 63 | 0.23 | >10000 |

Relative to PARP2 enzyme, most of the compounds tested have potent and selective inhibitory effect on PARP1 enzyme.

### Example 68

### Growth Inhibition Assays Against BRCA Mutant Human Breast Cancer MDA-MB-436 Cell Line

The cells were cultured in complete medium (DMEM medium+10% FBS+Insulin+glutathione). When the confluence reached about 80%, cells were digested and gently dispensed from the bottom of the dish with a 1 mL pipette. Cell suspension was collected and centrifuged at 500 rpm for 3 min. The supernatant was discarded, and the cell pellet were re-suspended in complete medium. The cells were seeded into a culture dish at an appropriate proportion, and then cultured in a 5% CO₂ incubator at 37° C. The assay was carried out when the cells were in optimum condition and the confluence was reached 80%. Cells in the logarithmic growth phase were taken to centrifugate, and the culture supernatant was removed. The cells were resuspended in refresh complete medium and counted. The resuspended cells were seeded at 3000/well in a 96-well plate and incubated at 37° C., 5% CO₂ incubator overnight. The compound was prepared as below: 1000× dilution tested compound solution to 40× test compound solution by adding 5 μL 1000× compound solution to 120 μL Medium (25-fold dilution). The solution was mixed by oscillation. 0.10% DMSO was used as the control.

The next day, the 96-well plate inoculated with cells was taken out from the incubator, and the culture supernatant was removed. Then fresh medium of 195 uL/well and 5 μL/well of 40× test compound solution as mentioned above were added into the 96 well plate, respectively. Finally, the plate was incubated for 7 days in a 37° C. 5% CO₂ incubator. The medium containing compound was changed on the fourth day. After 7 days, 20 μL of CCK-8 was added to each well and shaken gently, then was cultured for 4 hours. The plate was shaken for 5 min after incubation, the absorbance values of 450 nm or 650 nm wavelengths were recorded respectively (OD=absorbance value of 450 nm−absorbance value of 650 nm) by using the multifunction readout instrument.

Data were analyzed by software GraphPad Prism 6.0. The inhibitory activity of compounds on cell proliferation was plotted using cell survival rate against the compound concentration as coordinates. Cell survival rate %=(OD_{compound}−OD_{background})/(OD_{DMSO}−OD_{background})×100. The IC₅₀ value was fitted by the s-shaped dose response curve equation: Y=100/(1+10\hat{ }(log C-log IC₅₀)), and C was the compound concentration.

Table 2 summarizes the inhibitory effect data (IC₅₀) of the compounds on the proliferation of human breast cancer cells MDA-MB-436.

TABLE 2

| Example No. | IC₅₀ (nM) |
|---|---|
| 1-1 | 2.13 |
| 1-2 | >100 |
| 2 | 57 |
| 3 | 9.71 |
| 4 | 91 |
| 5 | >100 |
| 6 | 2.07 |
| 7 | 27.23 |
| 8 | 4.11 |
| 10-1 | >100 |
| 10-2 | >100 |
| 11 | >100 |
| 12 | >100 |
| 13 | 0.99 |
| 14 | 5.06 |
| 15 | 1.36 |
| 16 | 5.65 |
| 17 | >100 |
| 18 | 0.59 |
| 19 | 1.57 |
| 20 | 0.65 |
| 21 | 2.14 |
| 22 | >100 |
| 23 | >100 |
| 24 | >100 |
| 25 | 4.75 |
| 26 | 13.03 |
| 27 | 1.44 |
| 28 | 0.76 |
| 29 | 1.57 |
| 30 | 0.78 |
| 32 | 18.45 |
| 33 | >100 |
| 34 | 3.33 |
| 39 | 1.82 |
| 40 | >100 |
| 41 | 1.60 |
| 42 | 6.04 |
| 43 | 2.84 |
| 44 | >100 |
| 46 | 2.16 |
| 47 | 14.39 |
| 48 | 0.38 |
| 49 | 0.71 |
| 50 | 1.01 |
| 51 | 1.51 |
| 52 | 0.92 |
| 53 | 1.21 |
| 54 | 1.47 |
| 55 | 0.81 |
| 56 | 4.66 |
| 57 | 1.56 |
| 58 | 2.97 |
| 59 | 3.61 |
| 60 | >100 |
| 61 | >100 |
| 62 | 0.44 |
| 63 | 0.85 |
| 64 | >100 |

The compounds tested have good inhibitory effect on the proliferation of BRCA mutated human breast cancer cells MDA-MB-436.

### Example 69

### The Inhibitory Effect of the Compounds on PDE3A

A PDE3A fluorescence polarization (FP) assay in a multi-step format was performed using black, round bottom 384-well plates (Corning, 4514). The testing compounds were serially diluted from stock solutions to 11 concentrations with a 3-fold dilution factor using DMSO. Fifty nL of compound dilutions was mixed with 5 μL of reaction buffer (10 mM Tris-HCl, pH 7.2, 10 mM MgCl₂, 0.05% NaN₃, 0.1% phosphate-free BSA) containing 0.2 μM FAM-cAMP (BPS, 60200) and 2 nM PDE3A (Sino Biological, 11908-H20B1) enzyme, and incubated for 60 min at 25° C. Afterwards, 15 μL binding agent mixture (Molecular Devices, R8124) was added to each well and the plate was incubated for 60 min at 25° C. The plate was loaded to a BMG PHERAstar FSX to read Fluorescence Polarization (FP) values at a setting of: Ex: 485 nm and Em: 520 nm. Emission light intensity with polarizers parallel (Em_{|}) and emission light intensity with polarizers perpendicular (Em⊥) were recorded. The Polarization (mP) values were calculated using the following formula: mP=(Signal_{Em|}−Signal_{Em⊥})/(Signal_{Em|}+Signal_{Em⊥}).

Data analysis:
1) The inhibition % of a compound dose is calculated as:

$$\% Inh = ( {mP}_{Max} - {mP}_{cmpds} ) / ( {mP}_{Max} - {mP}_{Min} ) * 100 \%$$

wherein mP_{Min} and mP_{Max} refer to the polarization readings in the condition of substrate only and substrate+enzyme, respectively.
2) The IC₅₀ values were calculated from non-linear curve fitting using following regression equation using a commercial curve fitting software GraphPad Prism 9.2.0.

$$Y = Bottom + ( Top - Bottom ) / ( 1 + 10 \hat{} ( ( Log {IC}_{50} - X ) * Hillslope ) )$$

wherein X refers to Log of compound concentration and Y refers to % Inhibition.

Table 3 summarize the inhibitory effects of compounds on PDE3A enzyme activity (IC₅₀).

TABLE 3

| Example No. | IC₅₀ (μM) |
|---|---|
| 1-1 | >30 |
| 1-2 | >25 |
| 6 | >30 |
| 8 | >30 |
| 11 | >30 |
| 12 | >30 |
| 13 | >30 |
| 14 | >30 |
| 15 | >30 |
| 16 | >30 |
| 18 | >30 |
| 19 | >30 |
| 27 | >30 |
| 28 | 29.4 |
| 29 | >30 |
| 39 | >30 |
| 42 | >30 |
| 48 | >30 |
| 49 | >30 |
| 51 | >30 |
| 52 | >30 |
| 54 | >30 |
| 55 | >30 |
| 57 | >30 |
| 58 | 2.41 |
| 59 | >30 |

The results indicate that the compounds of this disclosure have low or even very low inhibitory effect on PDE3A (IC₅₀>30 μM).

Having now fully described this disclosure, it will be understood by those of ordinary skill in the art that the same can be performed within a wide and equivalent range of conditions, formulations and other parameters without affecting the scope of the disclosure or any embodiment thereof. All patents, patent applications and publications cited herein are fully incorporated by reference herein in their entirety.

What is claimed is:

1. A compound of Formula I: 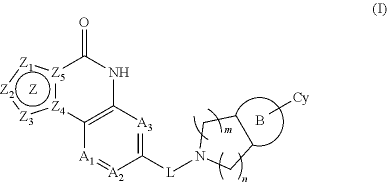
or stereoisomers, tautomers, N-oxides, hydrates, solvates, isotope-substituted derivatives, or pharmaceutically acceptable salts thereof, or prodrugs thereof, or mixtures thereof, wherein:
the Z ring shown as 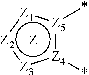
is an optionally substituted 5-membered heteroaryl group, * indicates the position at which the Z ring is attached to the rest of the compound, Z₁, Z₂ and Z₃ are each independently CR₁, NR₂, O, N or S, Z₄ and Z₅ are each independently C or N, and Z₄ and Z₅ are not N at the same time;
A₁, A₂ and A₃ are each independently selected from N and CR₃;
L is selected from a bond and an alkylene optionally substituted by R₄ and/or R₅;
the B ring is an optionally substituted heteroaryl or an optionally substituted heterocyclic group;
Cy is selected from a group consisting of an optionally substituted aryl and an optionally substituted heteroaryl;
R₁ is selected from a group consisting of hydrogen, halogen, hydroxyl, cyano, an optionally substituted alkyl, an optionally substituted alkoxy and an optionally substituted carbocyclic group;
R₂ is selected from a group consisting of hydrogen, an optionally substituted alkyl, an optionally substituted alkoxy, an optionally substituted cycloalkyl, an optionally substituted alkenyl and an optionally substituted alkynyl;
R₃ is selected from a group consisting of hydrogen, halogen, an optionally substituted alkyl, an optionally substituted alkoxy and an optionally substituted carbocyclic group;
R₄ and R₅ are each independently selected from a group consisting of halogen, cyano, an optionally substituted alkyl, an optionally substituted alkoxy, an optionally substituted cycloalkyl, an optionally substituted alkenyl, and an optionally substituted alkynyl; or R₄ and R₅ together with the attached C form a ring;
m and n are each independently selected from 1 and 2.

2. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein:
Z ring is selected from the following groups: 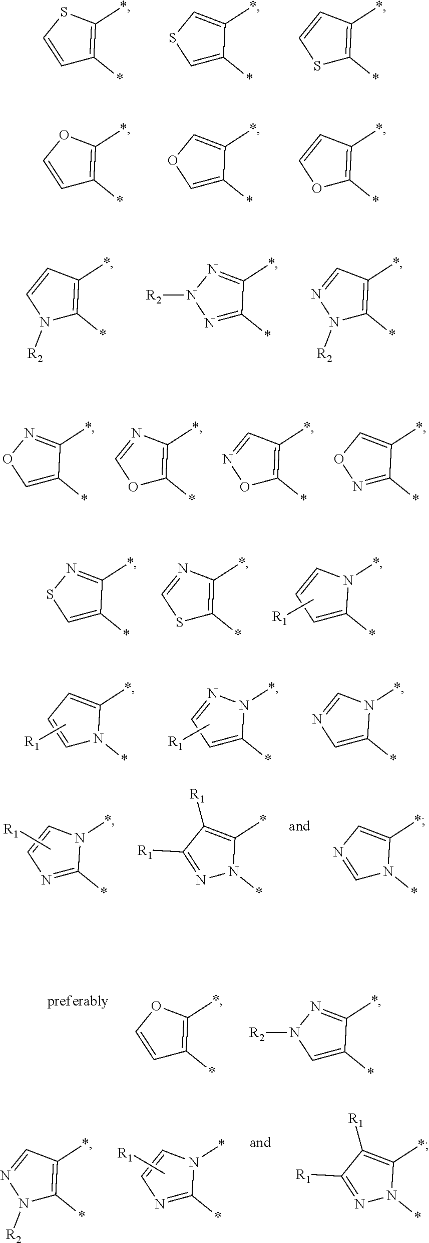
wherein * indicates the position at which the Z ring is attached to the rest of the compound.

3. The compound of claim 1 or 2, or stereoisomers, tautomers, N-oxides, hydrates, solvates, isotope-substituted derivatives, or pharmaceutically acceptable salts thereof, or mixtures thereof, or prodrugs thereof, wherein:
the said alkyl, alkoxy and carbocyclic group of R₁ are each optionally substituted by 1 to 5 groups selected from halogen, hydroxyl and —NR′R″, wherein R′ and R″ are each independently H or C₁₋₄ alkyl; preferably R₁ is hydrogen, halogen or an optionally substituted C₁₋₃ alkyl;
the said alkyl, alkoxy, cycloalkyl, alkenyl and alkynyl are each optionally substituted by 1 to 5 groups selected from halogen, hydroxyl and —NR′R″, wherein R′ and R″ are each independently H or C₁₋₄ alkyl; preferably R₂ is hydrogen, an optionally substituted C₁₋₃ alkyl and an optionally substituted C₃₋₆ cycloalkyl, more preferably hydrogen or an optionally substituted C₁₋₃ alkyl.

4. The compound of any one of claims 1-3, or stereoisomers, tautomers, N-oxides, hydrates, solvates, isotope-substituted derivatives, or pharmaceutically acceptable salts thereof, or mixtures thereof, or prodrugs thereof, wherein:
A₁, A₂ and A₃ are each independently selected from N and CR₃, wherein R₃ is preferably hydrogen, halogen, an optionally substituted C₁₋₃ alkyl or an optionally substituted C₁₋₃ alkoxy; preferably, the said optionally substituted C₁₋₃ alkyl and optionally substituted C₁₋₃ alkoxy can be optionally substituted by 1 to 5 groups selected from halogen, hydroxyl and —NR′R″, wherein R′ and R″ are each independently H or C₁₋₄ alkyl; preferably, R₃ is hydrogen, halogen or C₁₋₃ alkyl;
preferably, all of A₁, A₂ and A₃ are CR₃; wherein R₃ is each independently hydrogen, halogen or C₁₋₃ alkyl; or A₁ is CR₃, both of A₂ and A₃ are CH; or A₁ is CH, A₂ is CR₃, A₃ is CH; or both of A₁ and A₂ are CH, A₃ is CR₃; wherein R₃ is each independently halogen or C₁₋₃ alkyl; more preferably, both of A₁ and A₂ are CH, and A₃ is CR₃; preferably, R₃ is halogen, such as fluoro.

5. The compound of any one of claims 1-4, or stereoisomers, tautomers, N-oxides, hydrates, solvates, isotope-substituted derivatives, or pharmaceutically acceptable salts thereof, or mixtures thereof, or prodrugs thereof, wherein:
B ring is an optionally substituted 5- or 6-membered heteroaryl or an optionally substituted 5-6 membered heterocyclic group, preferably containing 1 or 2 heteroatoms selected from O, N and S; preferably, B ring is optionally substituted by 1-3 groups selected from a group consisting of halogen, amino, hydroxyl, optionally substituted C₃₋₈ cycloalkyl, optionally substituted C₁₋₆ alkyl; wherein the said optionally substituted C₃₋₈ cycloalkyl is optionally substituted by 1-3 groups selected from a group consisting of halogen, C₁₋₄ alkyl, C₁₋₄ alkoxy, halogenated C₁₋₄ alkyl, halogenated C₁₋₄ alkoxy, hydroxyl and amino; the said optionally substituted C₁₋₆ alkyl is optionally substituted by 1-3 groups selected from a group consisting of halogen, hydroxyl and amino;
preferably, 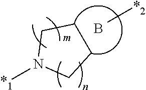
is selected from the following groups: 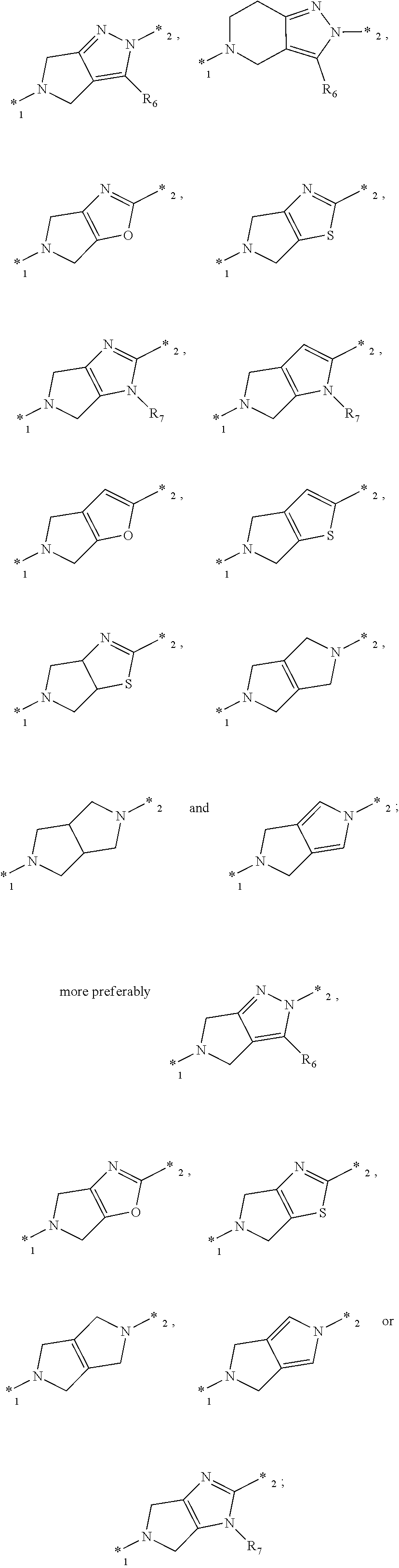
wherein, *1 indicates the position at which B ring is attached to L; *2 indicates the position at which B ring is attached to Cy; R₆ is selected from hydrogen, halogen, amino, hydroxyl and an optionally substituted C₁₋₃ alkyl; R₇ is selected from hydrogen, an optionally substituted C₁₋₃ alkyl and an optionally substituted C₃₋₆ cycloalkyl, preferably hydrogen or optionally substituted C₁₋₃ alkyl.

6. The compound of any one of claims 1-5, or stereoisomers, tautomers, N-oxides, hydrates, solvates, isotope-substituted derivatives, or pharmaceutically acceptable salts thereof, or mixtures thereof, or prodrugs thereof, wherein:
Cy is an 6-14 membered aryl or an 5-10 membered heteroaryl each independently and optionally substituted by 1-5 substituents selected from a group consisting of halogen, hydroxyl, cyano, an optionally substituted C₁₋₄ alkyl, an optionally substituted C₁₋₄ alkoxy, an optionally substituted C₂₋₄ alkenyl, an optionally substituted C₂₋₄ alkynyl, an optional substituted C₃₋₈ cycloalkyl, amino (—NR′R″), aminoacyl (—C(O)—NR′R″) and carboxyl, wherein the said R′ and R″ each are independently H, an optionally substituted C₁₋₁₀ alkyl, an optionally substituted C₃₋₈ cycloalkyl, an optionally substituted heterocyclic group, an optionally substituted aryl or an optionally substituted heteroaryl, preferably H, an optionally substituted C₁₋₄ alkyl, an optionally substituted C₃₋₆ cycloalkyl or an optionally substituted 3-6 membered heterocyclic group; preferably, the substituent(s) of Cy include at least —C(O)—NR′R″, and optionally include one or two of substituents selected from a group consisting of halogen, hydroxyl, cyano, C₁₋₄ alkyl, halogenated C₁₋₄ alkyl and C₃₋₆ cycloalkyl;
preferably, Cy is phenyl, pyridyl, pyrimidinyl, pyrazinyl or pyridazinyl optionally substituted by 1-5 substituents selected from a group consisting of halogen, hydroxyl, cyano, an optionally substituted C₁₋₄ alkyl, an optionally substituted C₁₋₄ alkoxy, C₃₋₆ cycloalkyl, aminoacyl and carboxyl; preferably, Cy is substituted by at least one aminoacyl group, preferably, the aminoacyl group is in the para position of Cy; preferably, the aminoacyl group is —C(O)—NR′R″; wherein the said R′ and R″ each are independently H, an optionally substituted C₁₋₁₀ alkyl, an optionally substituted C₃₋₈ cycloalkyl, an optionally substituted heterocyclic group, an optionally substituted aryl or an optionally substituted heteroaryl, preferably H, an optionally substituted C₁₋₄ alkyl, an optionally substituted C₃₋₆ cycloalkyl or an optionally substituted 3-6 membered heterocyclic group;
preferably, Cy is: 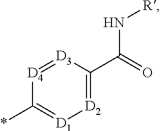
wherein, D₁, D₂, D₃ and D₄ are each independently selected from N and CR₈; R₈ is selected from a group consisting of hydrogen, halogen, hydroxyl, cyano, an optionally substituted C₁₋₄ alkyl, an optionally substituted C₁₋₄ alkoxy, an optionally substituted C₃₋₈ carbocyclic group, amino (—NR′R″), an optionally substituted C₂₋₄ alkenyl and an optionally substituted C₂₋₄ alkynyl; R′ is selected from an optionally substituted C₁₋₁₀ alkyl, an optionally substituted C₃₋₈ cycloalkyl, an optionally substituted heterocyclic group, an optionally substituted aryl or an optionally substituted heteroaryl, preferably an optionally substituted C₁₋₄ alkyl, an optionally substituted C₃₋₆ cycloalkyl or an optionally substituted 3-6 membered heterocyclic group; * indicates the position at which the said group is attached to the B ring; preferably, the ring containing D₁, D₂, D₃ and D₄ is phenyl, pyridyl, pyrimidinyl, pyrazinyl or pyridazinyl; preferably, R₈ is H, hydroxyl, halogen, cyano, C₁₋₃ alkyl, C₁₋₃ alkoxy, halogenated C₁₋₃ alkyl or C₃₋₆ cycloalkyl; preferably, D₃ is N, D₄ is CR₈, both of D₁ and D₂ are CH, wherein R₈ is H, hydroxyl, halogen, cyano, C₁₋₃ alkyl, C₁₋₃ alkoxy, halogenated C₁₋₃ alkyl or C₃₋₆ cycloalkyl.

7. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein the compound of Formula I is represented by Formula IIa, IIb, IIc or III as shown below: 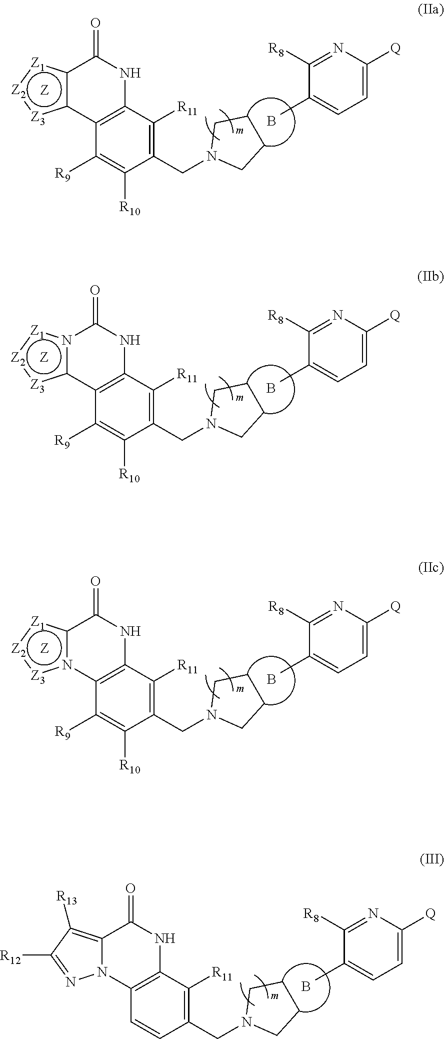
wherein Z₁, Z₂, Z₃ and Z ring are as defined in any one of claims 1-3;
m is as defined in claim 1;
B ring is as defined in claim 1 or 5;
R₈ is as defined in claim 6;
R₉, R₁₀ and R₁₁ are each independently selected from hydrogen, halogen, an optionally substituted C₁₋₃ alkyl and an optionally substituted C₁₋₃ alkoxy;
Q is cyano, an optionally substituted aminoacyl (—C(O)—NR′R″) or an optionally substituted heteroaryl, wherein R′ and R″ are each independently selected from hydrogen, an optionally substituted C₁₋₁₀ alkyl, an optionally substituted C₃₋₈ cycloalkyl, an optionally substituted heterocyclic group, an optionally substituted aryl and an optionally substituted heteroaryl;
R₁₂ and R₁₃ are each independently selected from hydrogen, halogen, cyano, an optionally substituted C₁₋₃ alkyl and an optionally substituted C₁₋₃ alkoxy.

8. The compound of claim 1, or stereoisomers, tautomers, N-oxides, hydrates, solvates, isotope-substituted derivatives, or pharmaceutically acceptable salts thereof, or mixtures thereof, or prodrugs thereof, wherein the Z ring is selected from the following groups: 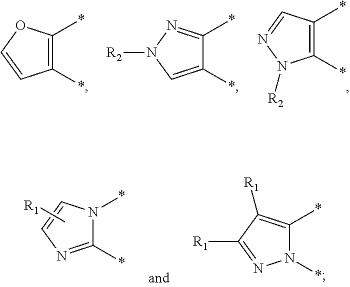
each R₁ is independently hydrogen, halogen, C₁₋₃ alkyl or halo C₁₋₃ alkyl; preferably, in 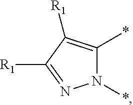
one of R₁ is hydrogen and another R₁ is halogen or C₁₋₃ alkyl;
R₂ is C₁₋₃ alkyl;
both of A₁ and A₂ are CH, A₃ is CR₃, wherein R₃ is halogen;
the said 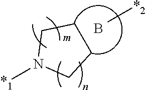
is selected from the following groups: 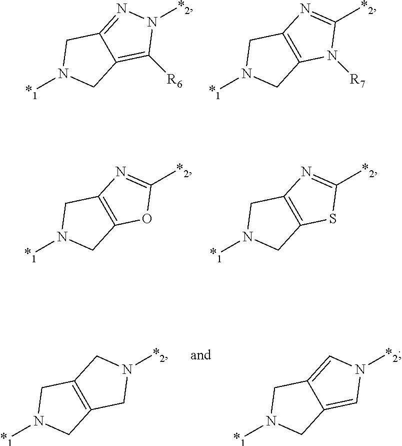
wherein, *1 indicates the position at which B ring is attached to L; *2 indicates the position at which B ring is attached to Cy; R₆ is hydrogen; R₇ is hydrogen;
Cy is: 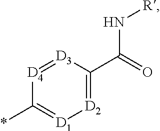
wherein D₃ is N, D₄ is CR₈, both of D₁ and D₂ are CH, wherein R₈ is H, C₁₋₃ alkyl or halogen; R′ is C₁₋₃ alkyl or C₃₋₆ cycloalkyl.

9. A compound selected from:
5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrazol-1(4H)-yl)-N-methylpicolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N,6-dimethylpicolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-6-fluoro-N-methylpicolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-6-chloro-N-methylpicolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-6-hydroxy-N-methylpicolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N-ethylpicolinamide;
N-ethyl-6-fluoro-5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)picolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)-N-cyclopropylpicolinamide;
N-cyclopropyl-6-fluoro-5-(5-((6-fluoro-3-methyl-4-oxo-4,5-dihydropyrazolo[1,5-a]quinoxalin-7-yl)methyl)-5,6-dihydropyrrolo[3,4-c]pyrazol-2(4H)-yl)picolinamide;
5-(5-((6-fluoro-3-methyl-4-oxo-4,5-dihydropyrazolo[1,5-a]quinoxalin-7-yl)methyl)-4,5,6,7-tetrahydro-2H-pyrazolo[4,3-c]pyridin-2-yl)-N-methylpicolinamide;
5-(5-((6-fluoro-3-methyl-4-oxo-4,5-dihydropyrazolo[1,5-a]quinoxalin-7-yl)methyl)-4,5,6,7-tetrahydro-1H-pyrazolo[4,3-c]pyridin-1-yl)-N-methylpicolinamide;
5-(5-((6-fluoro-3-methyl-4-oxo-4,5-dihydropyrazolo[1,5-a]quinoxalin-7-yl)methyl)-4,5,6,7-tetrahydro-2H-pyrazolo[4,3-c]pyridin-2-yl)-N,6-dimethylpicolinamide;
5-(5-((6-fluoro-3-methyl-4-oxo-4,5-dihydropyrazolo[1,5-a]quinoxalin-7-yl)methyl)-4,5,6,7-tetrahydro-2H-pyrazolo[4,3-c]pyridin-2-yl)-6-fluoro-N-methylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N,6-dimethylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-6-fluoro-N-methylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-6-chloro-N-methylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-6-hydroxy-N-methylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N-ethylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N-ethyl-6-fluoropicolinamide;
5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-N-cyclopropylpicolinamide;
N-cyclopropyl-5-(5-((3,6-difluoro-4-oxo-4,5-dihydropyrazolo[1,5-a]quinoxalin-7-yl)methyl)-5,6-dihydropyrrolo[3,4-c]pyrazol-2(4H)-yl)-6-fluoropicolinamide;
5-(5-((3,6-difluoro-4-oxo-4,5-dihydropyrazolo[1,5-a]quinoxalin-7-yl)methyl)-4,5,6,7-tetrahydro-2H-pyrazolo[4,3-c]pyridin-2-yl)-N-methylpicolinamide;
5-(5-((3,6-difluoro-4-oxo-4,5-dihydropyrazolo[1,5-a]quinoxalin-7-yl)methyl)-4,5,6,7-tetrahydro-2H-pyrazolo[4,3-c]pyridin-2-yl)-N,6-dimethylpicolinamide;
5-(5-((3,6-difluoro-4-oxo-4,5-dihydropyrazolo[1,5-a]quinoxalin-7-yl)methyl)-4,5,6,7-tetrahydro-2H-pyrazolo[4,3-c]pyridin-2-yl)-6-fluoro-N-methylpicolinamide;
5-(5-((6-fluoro-4-oxo-3-(trifluoromethyl)-4,5-dihydropyrazolo[1,5-a]quinoxalin-7-yl)methyl)-5,6-dihydropyrrolo[3,4-c]pyrazol-2(4H)-yl)-N-methylpicolinamide;
5-(5-((6-fluoro-4-oxo-3-(trifluoromethyl)-4,5-dihydropyrazolo[1,5-a]quinoxalin-7-yl)methyl)-5,6-dihydropyrrolo[3,4-c]pyrazol-2(4H)-yl)-6-fluoro-N-methylpicolinamide;
5-(5-((2,6-difluoro-4-oxo-4,5-dihydropyrazolo[1,5-a]quinoxalin-7-yl)methyl)-5,6-dihydropyrrolo[3,4-c]pyrazol-2(4H)-yl)-N-methylpicolinamide;
5-(5-((6-fluoro-4-oxo-4,5-dihydrofuro[2,3-c]quinolin-7-yl)methyl)-5,6-dihydropyrrolo[3,4-c]pyrazol-2(4H)-yl)-N-methylpicolinamide;
5-(5-((6-fluoro-2-methyl-4-oxo-4,5-dihydro-2H-pyrazolo[3,4-c]quinolin-7-yl)methyl)-5,6-dihydropyrrolo[3,4-c]pyrazol-2(4H)-yl)-N-methylpicolinamide;
5-(5-((6-fluoro-1-methyl-4-oxo-4,5-dihydro-1H-pyrazolo[4,3-c]quinolin-7-yl)methyl)-5,6-dihydropyrrolo[3,4-c]pyrazol-2(4H)-yl)-N-methylpicolinamide;
5-(5-((6-fluoro-3-methyl-4-oxo-4,5-dihydropyrazolo[1,5-a]quinoxalin-7-yl)methyl)-3-methyl-5,6-dihydropyrrolo[3,4-c]pyrazol-2(4H)-yl)-N-methylpicolinamide;
5-(5-((6-fluoro-3-methyl-4-oxo-4,5-dihydropyrazolo[1,5-a]quinoxalin-7-yl)methyl)-5,6-dihydro-4H-pyrrolo[3,4-d]oxazol-2-yl)-N-methylpicolinamide;
5-(5-((6-fluoro-3-methyl-4-oxo-4,5-dihydropyrazolo[1,5-a]quinoxalin-7-yl)methyl)-5,6-dihydro-4H-pyrrolo[3,4-d]thiazol-2-yl)-N-methylpicolinamide;
5-(5-((6-fluoro-3-methyl-4-oxo-4,5-dihydropyrazolo[1,5-a]quinoxalin-7-yl)methyl)-1,4,5,6-tetrahydropyrrolo[3,4-d]imidazol-2-yl)-N-methylpicolinamide;
5-(5-((6-fluoro-3-methyl-4-oxo-4,5-dihydropyrazolo[1,5-a]quinoxalin-7-yl)methyl)-1-methyl-1,4,5,6-tetrahydropyrrolo[3,4-d]imidazol-2-yl)-N-methylpicolinamide;
5-(5-((6-fluoro-3-methyl-4-oxo-4,5-dihydropyrazolo[1,5-a]quinoxalin-7-yl)methyl)-1-methyl-1,4,5,6-tetrahydropyrrolo[3,4-b]pyrrol-2-yl)-N-methylpicolinamide;
5-(5-((6-fluoro-3-methyl-4-oxo-4,5-dihydropyrazolo[1,5-a]quinoxalin-7-yl)methyl)-5,6-dihydro-4H-furo[2,3-c]pyrrol-2-yl)-N-methylpicolinamide;
5-(5-((6-fluoro-3-methyl-4-oxo-4,5-dihydropyrazolo[1,5-a]quinoxalin-7-yl)methyl)-5,6-dihydro-4H-thieno[2,3-c]pyrrol-2-yl)-N-methylpicolinamide;
5-(5-((6-fluoro-3-methyl-4-oxo-4,5-dihydropyrazolo[1,5-a]quinoxalin-7-yl)methyl)-5,6-dihydropyrrolo[3,4-c]pyrrol-2(4H)-yl)-N-methylpicolinamide;
5-(5-((3,6-difluoro-4-oxo-4,5-dihydropyrazolo[1,5-a]quinoxalin-7-yl)methyl)-3-methyl-5,6-dihydropyrrolo[3,4-c]pyrazol-2(4H)-yl)-N-methylpicolinamide;
5-(5-((3,6-difluoro-4-oxo-4,5-dihydropyrazolo[1,5-a]quinoxalin-7-yl)methyl)-5,6-dihydro-4H-pyrrolo[3,4-d]oxazol-2-yl)-N-methylpicolinamide;
5-(5-((3,6-difluoro-4-oxo-4,5-dihydropyrazolo[1,5-a]quinoxalin-7-yl)methyl)-5,6-dihydro-4H-pyrrolo[3,4-d]thiazol-2-yl)-N-methylpicolinamide;
5-(5-((3,6-difluoro-4-oxo-4,5-dihydropyrazolo[1,5-a]quinoxalin-7-yl)methyl)-1,4,5,6-tetrahydropyrrolo[3,4-d]imidazol-2-yl)-N-methylpicolinamide;
5-(5-((3,6-difluoro-4-oxo-4,5-dihydropyrazolo[1,5-a]quinoxalin-7-yl)methyl)-1-methyl-1,4,5,6-tetrahydropyrrolo[3,4-d]imidazol-2-yl)-N-methylpicolinamide;
5-(5-((3,6-difluoro-4-oxo-4,5-dihydropyrazolo[1,5-a]quinoxalin-7-yl)methyl)-1-methyl-1,4,5,6-tetrahydropyrrolo[3,4-b]pyrrol-2-yl)-N-methylpicolinamide;
5-(5-((3,6-difluoro-4-oxo-4,5-dihydropyrazolo[1,5-a]quinoxalin-7-yl)methyl)-5,6-dihydro-4H-furo[2,3-c]pyrrol-2-yl)-N-methylpicolinamide;
5-(5-((3,6-difluoro-4-oxo-4,5-dihydropyrazolo[1,5-a]quinoxalin-7-yl)methyl)-5,6-dihydro-4H-thieno[2,3-c]pyrrol-2-yl)-N-methylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrrol-2(4H)-yl)-N-methylpicolinamide;
5-(5-((7-fluoro-2-methyl-5-oxo-5,6-dihydroimidazo[1,2-c]quinazolin-8-yl)methyl)-5,6-dihydropyrrolo[3,4-c]pyrazol-2(4H)-yl)-N-methylpicolinamide;
N-ethyl-5-(5-((7-fluoro-2-methyl-5-oxo-5,6-dihydroimidazo[1,2-c]quinazolin-8-yl)methyl)-5,6-dihydropyrrolo[3,4-c]pyrazol-2(4H)-yl)picolinamide;
6-fluoro-5-(5-((7-fluoro-2-methyl-5-oxo-5,6-dihydroimidazo[1,2-c]quinazolin-8-yl)methyl)-5,6-dihydropyrrolo[3,4-c]pyrazol-2(4H)-yl)-N-methylpicolinamide;
N-ethyl-6-fluoro-5-(5-((7-fluoro-2-methyl-5-oxo-5,6-dihydroimidazo[1,2-c]quinazolin-8-yl)methyl)-5,6-dihydropyrrolo[3,4-c]pyrazol-2(4H)-yl)picolinamide;
N-ethyl-5-(5-((6-fluoro-4-oxo-4,5-dihydrofuro[2,3-c]quinolin-7-yl)methyl)-5,6-dihydropyrrolo[3,4-c]pyrazol-2(4H)-yl)picolinamide;
6-fluoro-5-(5-((6-fluoro-4-oxo-4,5-dihydrofuro[2,3-c]quinolin-7-yl)methyl)-5,6-dihydropyrrolo[3,4-c]pyrazol-2(4H)-yl)-N-methylpicolinamide;
N-ethyl-6-fluoro-5-(5-((6-fluoro-4-oxo-4,5-dihydrofuro[2,3-c]quinolin-7-yl)methyl)-5,6-dihydropyrrolo[3,4-c]pyrazol-2(4H)-yl)picolinamide;
5-(5-((2,6-difluoro-4-oxo-4,5-dihydropyrazolo[1,5-a]quinoxalin-7-yl)methyl)-5,6-dihydropyrrolo[3,4-c]pyrazol-2(4H)-yl)-6-fluoro-N-methylpicolinamide;
5-(5-((2,6-difluoro-4-oxo-4,5-dihydropyrazolo[1,5-a]quinoxalin-7-yl)methyl)-5,6-dihydropyrrolo[3,4-c]pyrazol-2(4H)-yl)-N-ethyl-6-fluoropicolinamide;
(R)-3-((3,6-difluoro-4-oxo-4,5-dihydropyrazolo[1,5-a]quinoxalin-7-yl)methyl)-N-methyl-1,2,3,4,4a,5-hexahydro-7H-pyrazino[2,1-c]pyrido[3,2-e][1,4]oxazepine-9-carboxamide;
5-(5-((3,6-difluoro-4-oxo-4,5-dihydropyrazolo[1,5-a]quinoxalin-7-yl)methyl)-3,4,5,6-tetrahydropyrrolo[3,4-c]pyrrol-2(1H)-yl)-6-fluoro-N-methylpicolinamide;
5-(5-((3,6-difluoro-4-oxo-4,5-dihydropyrazolo[1,5-a]quinoxalin-7-yl)methyl)-3a,5,6,6a-tetrahydro-4H-pyrrolo[3,4-d]thiazol-2-yl)-N-methylpicolinamide;
5-(5-((3,6-difluoro-4-oxo-4,5-dihydropyrazolo[1,5-a]quinoxalin-7-yl)methyl)hexahydropyrrolo[3,4-c]pyrrol-2(1H)-yl)-6-fluoro-N-methylpicolinamide;
5-(5-((3,6-difluoro-4-oxo-4,5-dihydropyrazolo[1,5-a]quinoxalin-7-yl)methyl)-5,6-dihydropyrrolo[3,4-c]pyrrol-2(4H)-yl)-6-fluoro-N-methylpicolinamide;
6-fluoro-5-(5-((6-fluoro-3-methyl-4-oxo-4,5-dihydropyrazolo[1,5-a]quinoxalin-7-yl)methyl)-5,6-dihydropyrrolo[3,4-c]pyrrol-2(4H)-yl)-N-methylpicolinamide;
6-fluoro-5-(5-((6-fluoro-3-methyl-4-oxo-4,5-dihydropyrazolo[1,5-a]quinoxalin-7-yl)methyl)hexahydropyrrolo[3,4-c]pyrrol-2(1H)-yl)-N-methylpicolinamide;
5-(5-((3,6-difluoro-4-oxo-4,5-dihydropyrazolo[1,5-a]quinoxalin-7-yl)methyl)-5,6-dihydro-4H-pyrrolo[3,4-d]oxazol-2-yl)-6-fluoro-N-methylpicolinamide;
5-(5-((3,6-difluoro-4-oxo-4,5-dihydropyrazolo[1,5-a]quinoxalin-7-yl)methyl)-5,6-dihydro-4H-furo[2,3-c]pyrrol-2-yl)-6-fluoro-N-methylpicolinamide;
or stereoisomers, tautomers, N-oxides, hydrates, isotope-substituted derivatives, solvates or pharmaceutically acceptable salts thereof, or mixtures thereof, or prodrugs thereof.

10. Use of the compound of any one of claims 1-9, or stereoisomers, tautomers, N-oxides, hydrates, isotope-substituted derivatives, solvates or pharmaceutically acceptable salts thereof, or prodrugs thereof, or mixtures thereof in the manufacture of a medicament for treatment or prevention of a disease or condition responsive to the inhibition of PARP activity;
preferably, the disease or condition is cancer; preferably, the cancer is selected from liver cancer, melanoma, Hodgkin's disease, non-Hodgkin's lymphomas, acute lymphocytic leukemia, chronic lymphocytic leukemia, multiple myeloma, neuroblastoma, breast cancer, ovarian cancer, lung cancer (such as small cell lung cancer), Wilms' tumor, cervical cancer, testicular cancer, soft-tissue sarcoma, primary macroglobulinemia, bladder cancer, chronic myeloid leukemia, primary brain cancer, malignant melanoma, gastric cancer, colon cancer, malignant pancreatic islet tumor, malignant carcinoid cancer, choriocarcinoma, mycosis fungoide, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, or prostatic cancer;
preferably, the medicament further comprises at least one known anticancer drug or a pharmaceutically acceptable salt thereof; preferably, the anticancer drug is selected from a group consisting of a group consisting of: vorinostat, romidepsin, panobinostat, belinostat, palbociclib, busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin, mabthera, T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin, abiraterone, darolutamide, enzalutamide, prednisone, imatinib, gefitinib, erlotinib, osimertinib, afatinib, ceritinib, alectinib, crizotinib, erlotinib, lafenib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, idelalisib, pazopanib, Torisel, everolimus, tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine);
preferably, the medicament is used in combination with radiotherapy.

11. A pharmaceutical composition comprising the compound of any one of claims 1-9, or stereoisomers, tautomers, N-oxides, hydrates, isotope-substituted derivatives, solvates or pharmaceutically acceptable salts thereof, or prodrugs thereof or mixtures thereof and a pharmaceutically acceptable carrier.

12. The pharmaceutical composition of claim 11, wherein the composition further includes at least one known anticancer drug or pharmaceutically acceptable salts thereof; preferably, the at least one known anticancer drug is selected from a group consisting of the group consisting of vorinostat, romidepsin, panobinostat, belinostat, palbociclib, busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin, mabthera, T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin, abiraterone, darolutamide, enzalutamide, prednisone, imatinib, gefitinib, erlotinib, ostinib, afatinib, ceritinib, alectinib, crizotinib, erlotinib, lapatinib, solutinib lafenib, regorafenib, vemurafenib, dabrafenib, aflibercept, sunitinib, nilotinib, dasatinib, bosutinib, pratinib, brutinib, cabozantinib, lenvatinib, vandetanib, trametinib, cabitinib, axitinib, temsirolimus, idelalisib, pazopanib, everolimus, tamoxifen, letrozole, fulvestrant, mitoguanhydrazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2), sipueucel-T (prostate cancer therapeutic vaccine).
